(19) United States
(12) Patent Application Publication
(10) Pub. No.: US 2025/0066339 A1
(43) Pub. Date: Feb. 27, 2025

(54) HETEROCYCLE-SUBSTITUTED AROMATIC COMPOUND, METHOD FOR PREPARING SAME, HERBICIDAL COMPOSITION, AND USE THEREOF

(71) Applicant: QINGDAO KINGAGROOT CHEMICAL COMPOUND CO., LTD., Qingdao, Shandong (CN)

(72) Inventors: Lei LIAN, Qingdao, Shandong (CN); Rongbao HUA, Qingdao, Shandong (CN); Xuegang PENG, Qingdao, Shandong (CN)

(73) Assignee: QINGDAO KINGAGROOT CHEMICAL COMPOUND CO., LTD., Qingdao, Shandong (CN)

(21) Appl. No.: 18/848,548

(22) PCT Filed: May 12, 2023
(86) PCT No.: PCT/CN2023/093933

§ 371 Date: Sep. 19, 2024

(30) Foreign Application Priority Data
May 13, 2022 (CN) 202210517156.1

(51) Int. Cl. C07D 413/10; A01N 43/80; A01N 43/84; A01N 47/36; A01P 13/00; C07D 405/14; C07D 409/14; C07D 413/14

(52) U.S. Cl. CPC C07D 413/10; A01N 43/80; A01N 43/84; A01N 47/36; A01P 13/00; C07D 405/14; C07D 409/14; C07D 413/14

(57) ABSTRACT

The present invention belongs to the technical field of pesticides, and particularly, relates to a heterocycle-substituted aromatic compound, a method for preparing same, a herbicidal composition, and use thereof. The compound is represented by general formula I, wherein Y represents a halogen, a halogenated alkyl, cyano, nitro, or amino; Z represents hydrogen, a halogen, or hydroxyl; M₁ and M₂ each independently represent CR₅ or N(O)ₘ; Q represents CX3X4; R₁, R₂, R₃, R₄, and R5 each independently represent hydrogen, a halogen, or the like; X₁, X₂, X₃, and X₄ each independently represent hydrogen, a halogen, or the like, and m represents 0 or 1. The compound, even at a low application rate, has excellent herbicidal activity on gramineous weeds, broad-leaf weeds, and the like, and has high selectivity for crops.

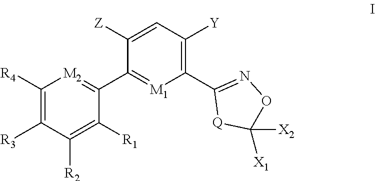

### TECHNICAL FIELD

The present invention belongs to the technical field of pesticides and particularly relates to a heterocycle-substituted aromatic compound, a preparation method therefor, and a herbicidal composition and an application thereof.

### BACKGROUND ART

Weed control is one of the most important links in the course of achieving high-efficiency agriculture. Although various herbicides are available in the market, for example, patents WO2012059050A and the like disclose the use of substituted biaryl benzenesulfonamide compounds as herbicides. However, the herbicidal properties against harmful plants and the selectivity to crops of these known compounds are not completely satisfactory. Moreover, due to the continuous expansion of the market, issues such as resistance of weeds, service life and economy of drugs, and the public's increasing attention to the environment, scientists are expected to continuously study and further develop new efficient, safe, and economical herbicide varieties with different modes of action.

### Contents of the Invention

The present invention provides a heterocycle-substituted aromatic compound, as well as a preparation method therefor, a herbicidal composition and an application thereof. The compound has excellent herbicidal activity against gramineous weeds, broad-leaf weeds and the like even at a low application rate and has high selectivity to crops.

The technical solution adopted in the present invention is as follows:

A heterocycle-substituted aromatic compound, as shown in the general formula I:

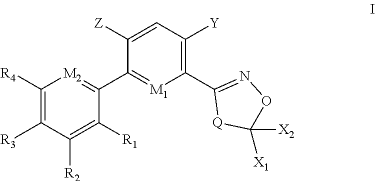

wherein,
Y represents halogen, haloalkyl, cyano, nitro, or amino;
Z represents hydrogen, halogen, or hydroxyl;
M₁ and M₂ each independently represent CR₅ or N(O)ₘ;
Q represents CX₃X₄;
R₁, R₂, R₃, R₄ and R₅ each independently represent hydrogen, halogen, hydroxyl, mercapto, formyl, hydroxylalkyl, nitro, cyano, alkyl, alkenyl, alkynyl, cycloalkyl, cycloalkylalkyl, cycloalkenyl, cycloalkenylalkyl, —OR₁₁, —SR₁₁, —(SO)R₁₁, —(SO₂)R₁₁, —(SO₂)OR₁₁, —O(SO₂)R₁₁, —N(R₁₂)₂, phenyl, or benzyl, wherein,
the “alkyl”, “alkenyl”, “alkynyl”, “cycloalkyl”, “cycloalkylalkyl”, “cycloalkenyl”, or “cycloalkenylalkyl” is each independently unsubstituted or substituted by halogen; the “phenyl” or “benzyl” is each independently unsubstituted or substituted by at least one group selected from halogen, cyano, nitro, alkyl, haloalkyl, alkoxycarbonyl, alkylsulfanyl, alkylsulfonyl, alkoxy, and haloalkoxy;
X₁, X₂, X₃, and X₄ each independently represent hydrogen, halogen, nitro, cyano, thiocyanato, hydroxyl, mercapto, sulfo, formyl, haloformyl, azido, alkyl, alkenyl, alkynyl, cycloalkyl, cycloalkylalkyl, cycloalkenyl, cycloalkenylalkyl, —PO(OR′)₂, —OR″, —(CO)R″, —SR″, —(SO)R″, —(SO₂)R″, —Si(R″)₃, —O(CO)R″, —O—(SO₂)R″, —S(CO)R″, —(SO₂)OR″, —O(CO)OR″, —(CO)(CO)OR″,

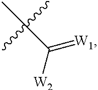

—CR′═N—OH, —CR′═N—O—R″, heterocyclyl, heterocyclylalkyl, aryl, arylalkyl, amino, aminoalkyl, aminocarbonylalkyl, aminocarbonyloxyalkyl, aminothiocarbonyloxyalkyl, aminosulfonyl, or aminosulfonyloxyalkyl, wherein,
the “alkyl”, “alkenyl”, or “alkynyl” is each independently unsubstituted or substituted by at least one group selected from halogen, cyano, hydroxyl, mercapto, carboxyl, —OR″, —(CO)R″, —SR″, —(SO₂)R″, —O(CO)H, —O(CO)R″, —O—(SO₂)R″, —(CO)OR″, —O(CO)OR″, —O(CO)(CO)OH, —O(CO)(CO)OR″, —O-alkyl-(CO)OH, and —O-alkyl-(CO)OR″;
the “cycloalkyl”, “cycloalkylalkyl”, “cycloalkenyl”, “cycloalkenylalkyl”, “heterocyclyl”, “heterocyclylalkyl”, “aryl”, or “arylalkyl” is each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₄, —SR₁₄, —(CO)OR₁₄, —(SO₂)R₁₄, —N(R₁₄)₂, and —O-alkyl-(CO)OR₁₄, or two adjacent carbon atoms on the ring form a fused ring with —OCH₂CH₂— or —OCH₂O— that is unsubstituted or substituted by halogen;
the “amino”, “aminoalkyl”, “aminocarbonylalkyl”, “aminocarbonyloxyalkyl”, “aminothiocarbonyloxyalkyl”, “aminosulfonyl”, or “aminosulfonyloxyalkyl” is each independently unsubstituted or substituted by one or two groups selected from —R₁₁, —OR₁₁, —(CO)R₁₁, —(CO)OR₁₁, -alkyl-(CO)OR₁₁, —(SO₂)R₁₁, —(SO₂)OR₁₁, -alkyl-(SO₂)R₁₁, —(CO)N(R₁₂)₂, and —(SO₂)N(R₁₂)₂;
R′ each independently represents hydrogen, halogen, alkoxy, alkoxyalkyl, alkyl, alkenyl, alkynyl, cycloalkyl, cycloalkylalkyl, cycloalkenyl, cycloalkenylalkyl, aryl, arylalkyl, heterocyclyl, or heterocyclylalkyl, wherein, the “alkyl”, “alkenyl”, or “alkynyl” is each independently unsubstituted or substituted by halogen; the “cycloalkyl”, “cycloalkylalkyl”, “cycloalkenyl”, “cycloalkenylalkyl”, “aryl”, “arylalkyl”, “heterocyclyl”, or “heterocyclylalkyl” is each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₄, —SR₁₄, —(CO)OR₁₄, —(SO₂)R₁₄, —N(R₁₄)₂, and —O-alkyl-(CO)OR₁₄, or two adjacent carbon atoms on the ring form a fused ring with —OCH₂CH₂— or —OCH₂O— that is unsubstituted or substituted by halogen;
R″ each independently represents alkyl, alkenyl, alkynyl, cycloalkyl, cycloalkylalkyl, cycloalkenyl, cycloalkenylalkyl, aryl, arylalkyl, arylalkenyl, heterocyclyl, heterocyclylalkyl, or heterocyclylalkenyl, wherein, the “alkyl”, “alkenyl”, or “alkynyl” is each independently unsubstituted or substituted by at least one group selected from halogen, cyano, trialkylsilyl, —OR₁₃, —SR₁₃, —O(CO)R₁₃, —(CO)R₁₃, —(CO)OR₁₃, and —O(CO)OR₁₃; the “cycloalkyl”, “cycloalkylalkyl”, “cycloalkenyl”, “cycloalkenylalkyl”, “aryl”, “arylalkyl”, “arylalkenyl”, “heterocyclyl”, “heterocycylalkyl”, or “heterocyclylalkenyl” is each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₄, —SR₁₄, —(CO)OR₁₄, —(SO₂)R₁₄, —N(R₁₄)₂ and —O-alkyl-(CO)OR₁₄, or two adjacent carbon atoms on the ring form a fused ring with —OCH₂CH₂— or —OCH₂O— that is unsubstituted or substituted by halogen;
W₁ represents O, S, or NW₄;
W₂ represents OW₃, SW₃, or NW₄W₅;
W₃ each independently represents hydrogen, alkyl, alkenyl, alkynyl, cycloalkyl, cycloalkenyl, heterocyclyl, aryl,

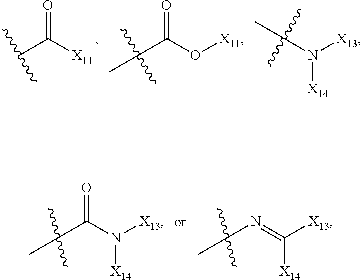

wherein, the “alkyl”, “alkenyl”, or “alkynyl” is each independently unsubstituted or substituted by at least one group selected from halogen, cyano, nitro, cycloalkyl, trialkylsilyl, cycloalkenyl, heterocyclyl, aryl,

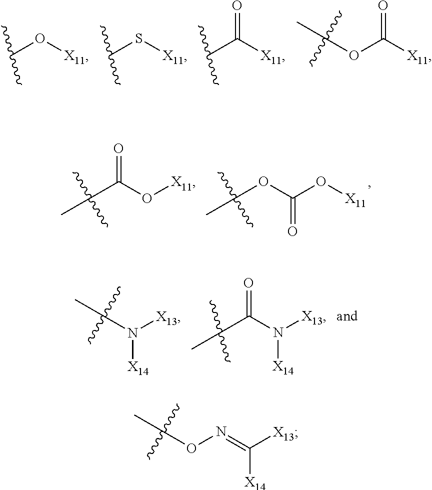

the “cycloalkyl”, “cycloalkenyl”, “heterocyclyl”, or “aryl” is each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₄, —SR₁₄, —(CO)OR₁₄, —(SO₂)R₁₄, —N(R₁₄)₂, and —O-alkyl-(CO)OR₁₄, or two adjacent carbon atoms on the ring form a fused ring with —OCH₂CH₂— or —OCH₂O— that is unsubstituted or substituted by halogen;
W₄ and W₅ each independently represent hydrogen, alkyl, alkenyl, alkynyl, cycloalkyl, cycloalkenyl, heterocyclyl, aryl,

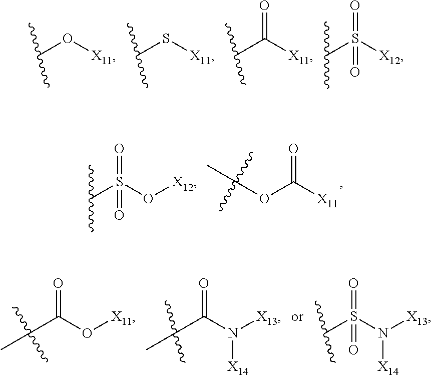

wherein, the “alkyl”, “alkenyl”, or “alkynyl” is each independently unsubstituted or substituted by at least one group selected from halogen, cyano, nitro, cycloalkyl, trialkylsilyl, cycloalkenyl, heterocyclyl, aryl,

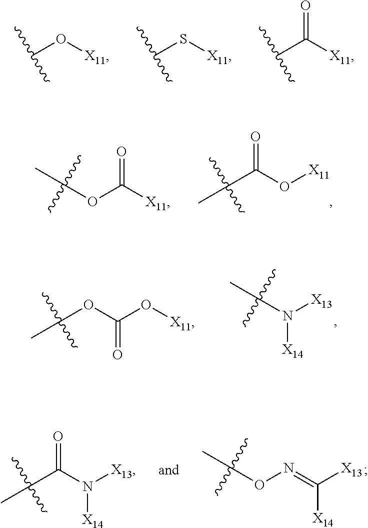

the “cycloalkyl”, “cycloalkenyl”, “heterocyclyl”, or “aryl” is each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₄, —SR₁₄, —(CO)OR₁₄, —(SO₂)R₁₄, —N(R₁₄)₂, and —O-alkyl-(CO)OR₁₄, or two adjacent carbon atoms on the ring form a fused ring with —OCH₂CH₂— or —OCH₂O— that is unsubstituted or substituted by halogen;
or NW₄W₅ represents

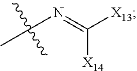

or unsubstituted or substituted heterocyclyl with a nitrogen atom at 1-position;
X₁₁ each independently represents hydrogen, alkyl, alkenyl, alkynyl, cycloalkyl, cycloalkylalkyl, cycloalkenyl, cycloalkenylalkyl, heterocyclyl, heterocyclylalkyl, aryl, or arylalkyl, wherein, the “cycloalkyl”, “cycloalkylalkyl”, “cycloalkenyl”, “cycloalkenylalkyl”, “heterocyclyl”, “heterocyclylalkyl”, “aryl”, or “arylalkyl” is each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₄, —SR₁₄, —(CO)OR₁₄, —(SO₂)R₁₄, —N(R₁₄)₂, and —O-alkyl-(CO)OR₁₄, or two adjacent carbon atoms on the ring form a fused ring with —OCH₂CH₂— or —OCH₂O— that is unsubstituted or substituted by halogen;
X₁₂ each independently represents alkyl, alkenyl, alkynyl, cycloalkyl, cycloalkylalkyl, cycloalkenyl, cycloalkenylalkyl, heterocyclyl, heterocyclylalkyl, aryl, or arylalkyl, wherein, the “cycloalkyl”, “cycloalkylalkyl”, “cycloalkenyl”, “cycloalkenylalkyl”, “heterocyclyl”, “heterocyclylalkyl”, “aryl” or “arylalkyl” is each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₄, —SR₁₄, —(CO)OR₁₄, —(SO₂)R₁₄, —N(R₁₄)₂, and —O-alkyl-(CO)OR₁₄, or two adjacent carbon atoms on the ring form a fused ring with —OCH₂CH₂— or —OCH₂O— that is unsubstituted or substituted by halogen;
X₁₃ and X₁₄ each independently represent hydrogen, halogen, cyano, alkoxy, alkoxyalkyl, alkylcarbonyl, alkoxycarbonyl, alkylsulfonyl, alkyl, alkenyl, alkynyl, cycloalkyl, cycloalkylalkyl, cycloalkenyl, cycloalkenylalkyl, aryl, arylalkyl, heterocyclyl, or heterocyclylalkyl, or the group CX₁₃X₁₄ together forms an unsubstituted or substituted cyclic structure, or the group NX₁₃X₁₄ together forms an unsubstituted or substituted heterocyclyl with a nitrogen atom at 1-position, wherein, the “alkyl”, “alkenyl”, or “alkynyl” is each independently unsubstituted or substituted by halogen; the “cycloalkyl”, “cycloalkylalkyl”, “cycloalkenyl”, “cycloalkenylalkyl”, “aryl”, “arylalkyl”, “heterocyclyl”, or “heterocyclylalkyl” is each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₄, —SR₁₄, —(CO)OR₁₄, —(SO₂)R₁₄, —N(R₁₄)₂, and —O-alkyl-(CO)OR₁₄, or two adjacent carbon atoms on the ring form a fused ring with —OCH₂CH₂— or —OCH₂O— that is unsubstituted or substituted by halogen;
R₁₁ each independently represents alkyl, alkenyl, alkynyl, cycloalkyl, cycloalkylalkyl, cycloalkenyl, cycloalkenylalkyl, phenyl, or benzyl, wherein, the “alkyl”, “alkenyl”, or “alkynyl” is each independently unsubstituted or substituted by halogen; the “phenyl” or “benzyl” is each independently unsubstituted or substituted by at least one group selected from halogen, cyano, nitro, alkyl, haloalkyl, alkoxycarbonyl, alkylsulfanyl, alkylsulfonyl, alkoxy, and haloalkoxy;
R₁₂ each independently represents hydrogen, alkyl, alkenyl, alkynyl, alkoxy, alkylsulfonyl, cycloalkyl, cycloalkylalkyl, cycloalkenyl, or cycloalkenylalkyl, or the group N(R₁₂)₂ in —(CO)N(R₁₂)₂ or —(SO₂)N(R₁₂)₂ each independently represents unsubstituted or substituted heterocyclyl with a nitrogen atom at 1-position;
R₁₃ each independently represents alkyl, alkenyl, alkynyl, cycloalkyl, phenyl, or phenyl that is substituted by at least one group selected from halogen, cyano, nitro, alkyl, haloalkyl, alkoxy, haloalkoxy, alkoxycarbonyl, alkylsulfanyl, alkylsulfonyl, and phenoxy that is substituted by at least one group selected from halogen, cyano, nitro, alkyl, haloalkyl, alkoxy, and haloalkoxy;
R₁₄ each independently represents hydrogen, alkyl, haloalkyl, phenyl, or phenyl that is substituted by at least one group selected from halogen, cyano, nitro, alkyl, haloalkyl, alkoxycarbonyl, alkylsulfanyl, alkylsulfonyl, alkoxy, and haloalkoxy;
m represents 0 or 1.

In one specific embodiment, Y represents halogen, halo C1-C8 alkyl, cyano, nitro, or amino.

In another specific embodiment, Y represents halogen, halo C1-C6 alkyl, cyano, nitro, or amino.

In one specific embodiment, R₁, R₂, R₃, R₄, and R₅ each independently represent hydrogen, halogen, hydroxyl, mercapto, formyl, hydroxyl C1-C8 alkyl, nitro, cyano, C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, —OR₁₁, —SR₁₁, —(SO)R₁₁, —(SO₂)R₁₁, —(SO₂)OR₁₁, —O(SO₂)R₁₁, —N(R₁₂)₂, phenyl, or benzyl, wherein,
the “C1-C8 alkyl”, “C2-C8 alkenyl”, “C2-C8 alkynyl”, “C3-C8 cycloalkyl”, “C3-C8 cycloalkyl C1-C8 alkyl”, “C3-C8 cycloalkenyl”, or “C3-C8 cycloalkenyl C1-C8 alkyl” is each independently unsubstituted or substituted by halogen; the “phenyl” or “benzyl” is each independently unsubstituted or substituted by at least one group selected from halogen, cyano, nitro, C1-C8 alkyl, halo C1-C8 alkyl, C1-C8 alkoxycarbonyl, C1-C8 alkylsulfanyl, C1-C8 alkylsulfonyl, C1-C8 alkoxy, and halo C1-C8 alkoxy.

In another specific embodiment, R₁, R₂, R₃, R₄, and R₅ each independently represent hydrogen, halogen, hydroxyl, mercapto, formyl, hydroxyl C1-C6 alkyl, nitro, cyano, C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, —OR₁₁, —SR₁₁, —(SO)R₁₁, —(SO₂)R₁₁, —(SO₂)OR₁₁, —O(SO₂)R₁₁, —N(R₁₂)₂, phenyl, or benzyl, wherein,
the “C1-C6 alkyl”, “C2-C6 alkenyl”, “C2-C6 alkynyl”, “C3-C6 cycloalkyl”, “C3-C6 cycloalkyl C1-C6 alkyl”, “C3-C6 cycloalkenyl”, or “C3-C6 cycloalkenyl C1-C6 alkyl” is each independently unsubstituted or substituted by halogen; the “phenyl” or “benzyl” is each independently unsubstituted or substituted by 1, 2, or 3 groups selected from halogen, cyano, nitro, C1-C6 alkyl, halo C1-C6 alkyl, C1-C6 alkoxycarbonyl, C1-C6 alkylsulfanyl, C1-C6 alkylsulfonyl, C1-C6 alkoxy, and halo C1-C6 alkoxy.

In another specific embodiment, R₁, R₂, R₃, R₄, and R₅ each independently represent hydrogen, halogen, hydroxyl, mercapto, formyl, hydroxyl C1-C6 alkyl, nitro, cyano, C1-C6 alkyl, C2-C6 alkenyl, C2-C6 alkynyl, C3-C6 cycloalkyl, C3-C6 cycloalkyl C1-C3 alkyl, C3-C6 cycloalkenyl, C3-C6 cycloalkenyl C1-C3 alkyl, —OR₁₁, —SR₁₁, —(SO)R₁₁, —(SO₂)R₁₁, —(SO₂)OR₁₁, —O(SO₂)R₁₁, —N(R₁₂)₂, phenyl, or benzyl, wherein,
the “C1-C6 alkyl”, “C2-C6 alkenyl”, “C2-C6 alkynyl”, “C3-C6 cycloalkyl”, “C3-C6 cycloalkyl C1-C3 alkyl”, “C3-C6 cycloalkenyl”, or “C3-C6 cycloalkenyl C1-C3 alkyl” is each independently unsubstituted or substituted by halogen; the “phenyl” or “benzyl” is each independently unsubstituted or substituted by 1, 2, or 3 groups selected from halogen, cyano, nitro, C1-C6 alkyl, halo C1-C6 alkyl, C1-C6 alkoxycarbonyl, C1-C6 alkylsulfanyl, C1-C6 alkylsulfonyl, C1-C6 alkoxy, and halo C1-C6 alkoxy.

In one specific embodiment, X₁, X₂, X₃, and X₄ each independently represent hydrogen, halogen, nitro, cyano, thiocyanato, hydroxyl, mercapto, sulfo, formyl, haloformyl, azido, C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, —PO(OR′)₂, —OR″, —(CO)R″, —SR″, —(SO)R″, —(SO₂)R″, —Si R″)₃, —O(CO)R″, —O—(SO₂)R″, —S(CO)R″, —(SO₂)OR″, —O(CO)OR″, —(CO)(CO)OR″,

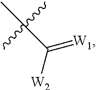

—CR′═N—OH, —CR′═N—O—R″, heterocyclyl, heterocyclyl C1-C8 alkyl, aryl, aryl C1-C8 alkyl, amino, amino C1-C8 alkyl, aminocarbonyl C1-C8 alkyl, aminocarbonyloxy C1-C8 alkyl, aminothiocarbonyloxy C1-C8 alkyl, aminosulfonyl, or aminosulfonyloxy C1-C8 alkyl, wherein,
the “C1-C8 alkyl”, “C2-C8 alkenyl”, or “C2-C8 alkynyl” is each independently unsubstituted or substituted by at least one group selected from halogen, cyano, hydroxyl, mercapto, carboxyl, —OR″, —(CO)R″, —SR″, —(SO₂)R″, —O(CO)H, —O(CO)R″, —O—(SO₂)R″, —(CO)OR″, —O(CO)OR″, —O(CO)(CO)OH, —O(CO)(CO)OR″, —O—(C1-C8 alkyl)-(CO)OH, and —O—(C1-C8 alkyl)-(CO)OR″;
the “C3-C8 cycloalkyl”, “C3-C8 cycloalkyl C1-C8 alkyl”, “C3-C8 cycloalkenyl”, “C3-C8 cycloalkenyl C1-C8 alkyl”, “heterocyclyl”, “heterocyclyl C1-C8 alkyl”, “aryl”, or “aryl C1-C8 alkyl” is each unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₄, —SR₁₄, —(CO)OR₁₄, —(SO₂)R₁₄, —N(R₁₄)₂, and —O—(C1-C8 alkyl)-(CO)OR₁₄, or two adjacent carbon atoms on the ring form a fused ring with —OCH₂CH₂— or —OCH₂O— that is unsubstituted or substituted by halogen;
the “amino”, “amino C1-C8 alkyl”, “aminocarbonyl C1-C8 alkyl”, “aminocarbonyloxy C1-C8 alkyl”, “aminothiocarbonyloxy C1-C8 alkyl”, “aminosulfonyl”, or “aminosulfonyloxy C1-C8 alkyl” is each independently unsubstituted or substituted by one or two groups selected from —R₁₁, —OR₁₁, —(CO)R₁₁, —(CO)OR₁₁, —C1-C8 alkyl)-(CO)OR₁₁, —(SO₂)R₁₁, —(SO₂)OR₁₁, —C1-C8 alkyl)-(SO₂)R₁₁, —(CO)N(R₁₂)₂, and —(SO₂)N(R₁₂)₂;
R′ each independently represents hydrogen, halogen, C1-C8 alkoxy, C1-C8 alkoxy C1-C8 alkyl, C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, aryl, aryl C1-C8 alkyl, heterocyclyl, or heterocyclyl C1-C8 alkyl, wherein, the “C1-C8 alkyl”, “C2-C8 alkenyl”, or “C2-C8 alkynyl” is each independently unsubstituted or substituted by halogen; the “C3-C8 cycloalkyl”, “C3-C8 cycloalkyl C1-C8 alkyl”, “C3-C8 cycloalkenyl”, “C3-C8 cycloalkenyl C1-C8 alkyl”, “aryl”, “aryl C1-C8 alkyl”, “heterocyclyl”, or “heterocyclyl C1-C8 alkyl” is each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₄, —SR₁₄, —(CO)OR₁₄, —(SO₂)R₁₄, —N(R₁₄)₂, and —O—(C1-C8 alkyl)-(CO)OR₁₄, or two adjacent carbon atoms on the ring form a fused ring with —OCH₂CH₂— or —OCH₂O— that is unsubstituted or substituted by halogen;
R″ each independently represents C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, aryl, aryl C1-C8 alkyl, aryl C2-C8 alkenyl, heterocyclyl, heterocyclyl C1-C8 alkyl, or heterocyclyl C2-C8 alkenyl, wherein, the “C1-C8 alkyl”, “C2-C8 alkenyl”, or “C2-C8 alkynyl” is each independently unsubstituted or substituted by at least one group selected from halogen, cyano, tri-C1-C8 alkylsilyl, —OR₁₃, —SR₁₃, —O(CO)R₁₃, —(CO)R₁₃, —(CO)OR₁₃, and O(CO)OR₁₃; the “C3-C8 cycloalkyl”, “C3-C8 cycloalkyl C1-C8 alkyl”, “C3-C8 cycloalkenyl”, “C3-C8 cycloalkenyl C1-C8 alkyl”, “aryl”, “aryl C1-C8 alkyl”, “aryl C2-C8 alkenyl”, “heterocyclyl”, “heterocyclyl C1-C8 alkyl”, or “heterocyclyl C2-C8 alkenyl” is each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₄, —SR₁₄, —(CO)OR₁₄, —(SO₂)R₁₄, —N(R₁₄)₂, and —O—(C1-C8 alkyl)-(CO)OR₁₄, or two adjacent carbon atoms on the ring form a fused ring with —OCH₂CH₂— or —OCH₂O— that is unsubstituted or substituted by halogen.

In another specific embodiment, X₁, X₂, X₃, and X₄ each independently represent hydrogen, halogen, nitro, cyano, thiocyanato, hydroxyl, mercapto, sulfo, formyl, haloformyl, azido, C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, —PO(OR′)₂, —OR″, —(CO)R″, —SR″, —(SO)R″, —(SO₂)R″, —Si(R″)₃, —O(CO)R″, —O—(SO₂)R″, —S(CO)R″, —(SO₂)OR″, —O(CO)OR″, —(CO)(CO)OR″,

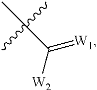

—CR′═N—OH, —CR′═N—O—R″, heterocyclyl, heterocyclyl C1-C6 alkyl, aryl, aryl C1-C6 alkyl, amino, amino C1-C6 alkyl, aminocarbonyl C1-C6 alkyl, aminocarbonyloxy C1-C6 alkyl, aminothiocarbonyloxy C1-C6 alkyl, aminosulfonyl, or aminosulfonyloxy C1-C6 alkyl, wherein,
the “C1-C6 alkyl”, “C2-C6 alkenyl”, or “C2-C6 alkynyl” is each independently unsubstituted or substituted by 1, 2, or 3 groups selected from halogen, cyano, hydroxyl, mercapto, carboxyl, —OR″, —(CO)R″, —SR″, —(SO₂)R″, —O(CO)H, —O(CO)R″, —O—(SO₂)R″, —(CO)OR″, —O(CO)OR″, —O(CO)(CO)OH, —O(CO)(CO)OR″, —O—(C1-C6 alkyl)-(CO)OH, and —O—(C1-C6 alkyl)-(CO)OR″;
the “C3-C6 cycloalkyl”, “C3-C6 cycloalkyl C1-C6 alkyl”, “C3-C6 cycloalkenyl”, “C3-C6 cycloalkenyl C1-C6 alkyl”, “heterocyclyl”, “heterocyclyl C1-C6 alkyl”, “aryl”, or “aryl C1-C6 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C6 alkyl)-(CO)OR₁₄, or two adjacent carbon atoms on the ring form a fused ring with —OCH₂CH₂— or —OCH₂O— that is unsubstituted or substituted by halogen;
the “amino”, “amino C1-C6 alkyl”, “aminocarbonyl C1-C6 alkyl”, “aminocarbonyloxy C1-C6 alkyl”, “aminothiocarbonyloxy C1-C6 alkyl”, “aminosulfonyl”, or “aminosulfonyloxy C1-C6 alkyl” is each independently unsubstituted or substituted by one or two groups selected from —R₁₁, —OR₁₁, —(CO)R₁₁, —(CO)OR₁₁, —C1-C6 alkyl)-(CO)OR₁₁, —(SO₂)R₁₁, —(SO₂)OR₁₁, —C1-C6 alkyl)-(SO₂)R₁₁, —(CO)N(R₁₂)₂, and —(SO₂)N(R₁₂)₂;

R′ each independently represents hydrogen, halogen, C1-C6 alkoxy, C1-C6 alkoxy C1-C6 alkyl, C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, aryl, aryl C1-C6 alkyl, heterocyclyl, or heterocyclyl C1-C6 alkyl, wherein, the “C1-C6 alkyl”, “C2-C6 alkenyl”, or “C2-C6 alkynyl” is each independently unsubstituted or substituted by halogen; the “C3-C6 cycloalkyl”, “C3-C6 cycloalkyl C1-C6 alkyl”, “C3-C6 cycloalkenyl”, “C3-C6 cycloalkenyl C1-C6 alkyl”, “aryl”, “aryl C1-C6 alkyl”, “heterocyclyl”, or “heterocyclyl C1-C6 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C6 alkyl)-(CO)OR₁₄, or two adjacent carbon atoms on the ring form a fused ring with —OCH₂CH₂— or —OCH₂O— that is unsubstituted or substituted by halogen; R″ each independently represents C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, aryl, aryl C1-C6 alkyl, aryl C2-C6 alkenyl, heterocyclyl, heterocyclyl C1-C6 alkyl, or heterocyclyl C2-C6 alkenyl, wherein, the “C1-C6 alkyl”, “C2-C6 alkenyl”, or “C2-C6 alkynyl” is each independently unsubstituted or substituted by 1, 2, or 3 groups selected from halogen, cyano, tri-C1-C6 alkylsilyl, —OR₁₃, —SR₁₃, —O(CO)R₁₃, —(CO)R₁₃, —(CO)OR₁₃, and —O(CO)OR₁₃; the “C3-C6 cycloalkyl”, “C3-C6 cycloalkyl C1-C6 alkyl”, “C3-C6 cycloalkenyl”, “C3-C6 cycloalkenyl C1-C6 alkyl”, “aryl”, “aryl C1-C6 alkyl”, “aryl C2-C6 alkenyl”, “heterocyclyl”, “heterocyclyl C1-C6 alkyl”, or “heterocyclyl C2-C6 alkenyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C6 alkyl)-(CO)OR₁₄, or two adjacent carbon atoms on the ring form a fused ring with —OCH₂CH₂— or —OCH₂O— that is unsubstituted or substituted by halogen.

In another specific embodiment, X₁, X₂, X₃, and X₄ each independently represent hydrogen, halogen, nitro, cyano, thiocyanato, hydroxyl, mercapto, sulfo, formyl, haloformyl, azido, C1-C6 alkyl, C2-C6 alkenyl, C2-C6 alkynyl, C3-C6 cycloalkyl, C3-C6 cycloalkyl C1-C3 alkyl, C3-C6 cycloalkenyl, C3-C6 cycloalkenyl C1-C3 alkyl, —PO(OR′)₂, —OR″, —(CO)R″, —SR″, —(SO)R″, —(SO₂)R″, —Si(R″)₃, —O(CO)R″, —O—(SO₂)R″, —S(CO)R″, —(SO₂)OR″, —O(CO)OR″, —(CO)(CO)OR″,

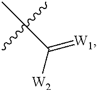

—CR′═N—OH, —CR′═N—O—R″, heterocyclyl, heterocyclyl C1-C3 alkyl, aryl, aryl C1-C3 alkyl, amino, amino C1-C3 alkyl, aminocarbonyl C1-C3 alkyl, aminocarbonyloxy C1-C3 alkyl, aminothiocarbonyloxy C1-C3 alkyl, aminosulfonyl, or aminosulfonyloxy C1-C3 alkyl, wherein,
the “C1-C6 alkyl”, “C2-C6 alkenyl”, or “C2-C6 alkynyl” is each independently unsubstituted or substituted by 1, 2, or 3 groups selected from halogen, cyano, hydroxyl, mercapto, carboxyl, —OR″, —(CO)R″, —SR″, —(SO₂)R″, —O(CO)H, —O(CO)R″, —O—(SO₂)R″, —(CO)OR″, —O(CO)OR″, —O(CO)(CO)OH, —O(CO)(CO)OR″, —O—(C1-C3 alkyl)-(CO)OH, and —O—(C1-C3 alkyl)-(CO)OR″;
the “C3-C6 cycloalkyl”, “C3-C6 cycloalkyl C1-C3 alkyl”, “C3-C6 cycloalkenyl”, “C3-C6 cycloalkenyl C1-C3 alkyl”, “heterocyclyl”, “heterocyclyl C1-C3 alkyl”, “aryl”, or “aryl C1-C3 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C3 alkyl)-(CO)OR₁₄, or two adjacent carbon atoms on the ring form a fused ring with —OCH₂CH₂— or —OCH₂O— that is unsubstituted or substituted by halogen;
the “amino”, “amino C1-C3 alkyl”, “aminocarbonyl C1-C3 alkyl”, “aminocarbonyloxy C1-C3 alkyl”, “aminothiocarbonyloxy C1-C3 alkyl”, “aminosulfonyl”, or “aminosulfonyloxy C1-C3 alkyl” is each independently unsubstituted or substituted by one or two groups selected from —R₁₁, —OR₁₁, —(CO)R₁₁, —(CO)OR₁₁, —C1-C3 alkyl)-(CO)OR₁₁, —(SO₂)R₁₁, —(SO₂)OR₁₁, —C1-C3 alkyl)-(SO₂)R₁₁, —(CO)N(R₁₂)₂, and —(SO₂)N(R₁₂)₂;
R′ each independently represents hydrogen, halogen, C1-C6 alkoxy, C1-C6 alkoxy C1-C3 alkyl, C1-C6 alkyl, C2-C6 alkenyl, C2-C6 alkynyl, C3-C6 cycloalkyl, C3-C6 cycloalkyl C1-C3 alkyl, C3-C6 cycloalkenyl, C3-C6 cycloalkenyl C1-C3 alkyl, aryl, aryl C1-C3 alkyl, heterocyclyl, or heterocyclyl C1-C3 alkyl, wherein, the “C1-C6 alkyl”, “C2-C6 alkenyl”, or “C2-C6 alkynyl” is each independently unsubstituted or substituted by halogen; the “C3-C6 cycloalkyl”, “C3-C6 cycloalkyl C1-C3 alkyl”, “C3-C6 cycloalkenyl”, “C3-C6 cycloalkenyl C1-C3 alkyl”, “aryl”, “aryl C1-C3 alkyl”, “heterocyclyl”, or “heterocyclyl C1-C3 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C3 alkyl)-(CO)OR₁₄, or two adjacent carbon atoms on the ring form a fused ring with —OCH₂CH₂— or —OCH₂O— that is unsubstituted or substituted by halogen;
R″ each independently represents C1-C6 alkyl, C2-C6 alkenyl, C2-C6 alkynyl, C3-C6 cycloalkyl, C3-C6 cycloalkyl C1-C3 alkyl, C3-C6 cycloalkenyl, C3-C6 cycloalkenyl C1-C3 alkyl, aryl, aryl C1-C3 alkyl, aryl C2-C3 alkenyl, heterocyclyl, heterocyclyl C1-C3 alkyl, or heterocyclyl C2-C3 alkenyl, wherein, the “C1-C6 alkyl”, “C2-C6 alkenyl”, or “C2-C6 alkynyl” is each independently unsubstituted or substituted by 1, 2, or 3 groups selected from halogen, cyano, tri-C1-C6 alkylsilyl, —OR₁₃, —SR₁₃, —O(CO)R₁₃, —(CO)R₁₃, —(CO)OR₁₃, and —O(CO)OR₁₃; the “C3-C6 cycloalkyl”, “C3-C6 cycloalkyl C1-C3 alkyl”, “C3-C6 cycloalkenyl”, “C3-C6 cycloalkenyl C1-C3 alkyl”, “aryl”, “aryl C1-C3 alkyl”, “aryl C2-C3 alkenyl”, “heterocyclyl”, “heterocyclyl C1-C3 alkyl”, or “heterocyclyl C2-C3 alkenyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C3 alkyl)-(CO)OR₁₄, or two adjacent carbon atoms on the ring form a fused ring with —OCH₂CH₂— or —OCH₂O— that is unsubstituted or substituted by halogen.

In one specific embodiment, W₃ each independently represents hydrogen, C1-C8 alkyl, C2-C8 alkenyl, C2-C8 alkynyl, C3-C8 cycloalkyl, C3-C8 cycloalkenyl, heterocyclyl, aryl,

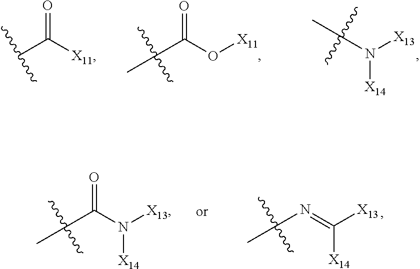

the “C1-C8 alkyl”, “C2-C8 alkenyl”, or “C2-C8 alkynyl” is each independently unsubstituted or substituted by at least one group selected from halogen, cyano, nitro, C3-C8 cycloalkyl, tri-C1-C8 alkylsilyl, C3-C8 cycloalkenyl, heterocyclyl, aryl,

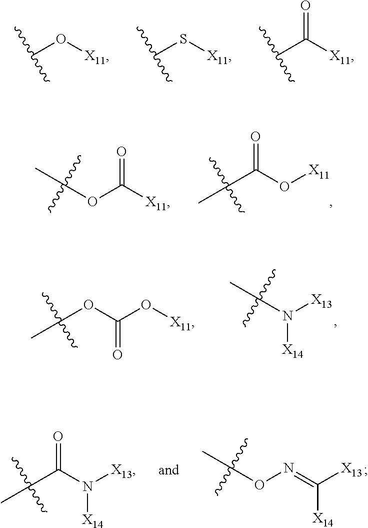

the “C3-C8 cycloalkyl”, “C3-C8 cycloalkenyl”, “heterocyclyl”, or “aryl” is each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₄, —SR₁₄, —(CO)OR₁₄, —(SO₂)R₁₄, —N(R₁₄)₂, and —O—(C1-C8 alkyl)-(CO)OR₁₄, or two adjacent carbon atoms on the ring form a fused ring with —OCH₂CH₂— or —OCH₂O— that is unsubstituted or substituted by halogen;

W₄ and W₅ each independently represent hydrogen, C1-C8 alkyl, C2-C8 alkenyl, C2-C8 alkynyl, C3-C8 cycloalkyl, C3-C8 cycloalkenyl, heterocyclyl, aryl,

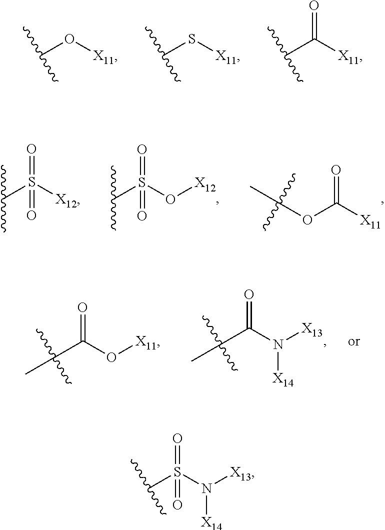

wherein, the “C1-C8 alkyl”, “C2-C8 alkenyl”, or “C2-C8 alkynyl” is each independently unsubstituted or substituted by at least one group selected from halogen, cyano, nitro, C3-C8 cycloalkyl, tri-C1-C8 alkylsilyl, C3-C8 cycloalkenyl, heterocyclyl, aryl,

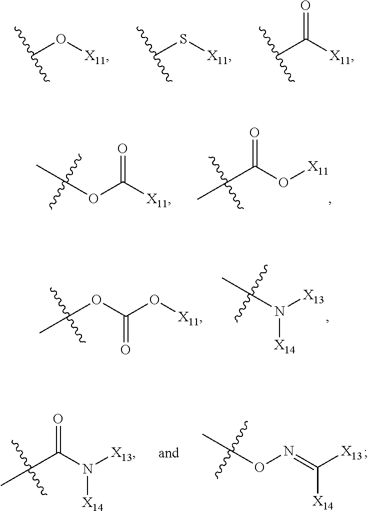

the “C3-C8 cycloalkyl”, “C3-C8 cycloalkenyl”, “heterocyclyl”, or “aryl” is each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₄, —SR₁₄, —(CO)OR₁₄, —(SO₂)R₁₄, —N(R₁₄)₂, and —O—(C1-C8 alkyl)-(CO)OR₁₄, or two adjacent carbon atoms on the ring form a fused ring with —OCH₂CH₂— or —OCH₂O— that is unsubstituted or substituted by halogen;
or NW₄W₅ represents

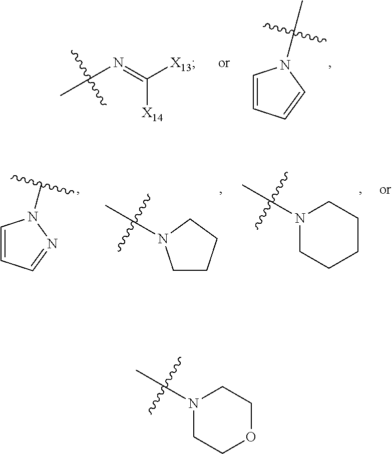

which is unsubstituted or substituted by at least one group selected from oxo, C1-C8 alkyl, and C1-C8 alkoxycarbonyl;

X₁₁ each independently represents hydrogen, C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, heterocyclyl, heterocyclyl C1-C8 alkyl, aryl, or aryl C1-C8 alkyl, wherein, the “C3-C8 cycloalkyl”, “C3-C8 cycloalkyl C1-C8 alkyl”, “C3-C8 cycloalkenyl”, “C3-C8 cycloalkenyl C1-C8 alkyl”, “heterocyclyl”, “heterocyclyl C1-C8 alkyl”, “aryl”, or “aryl C1-C8 alkyl” is each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₄, —SR₁₄, —(CO)OR₁₄, —(SO₂)R₁₄, —N(R₁₄)₂, and —O—(C1-C8 alkyl)-(CO)OR₁₄, or two adjacent carbon atoms on the ring form a fused ring with —OCH₂CH₂— or —OCH₂O— that is unsubstituted or substituted by halogen;

X₁₂ each independently represents C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, heterocyclyl, heterocyclyl C1-C8 alkyl, aryl, or aryl C1-C8 alkyl, wherein, the “C3-C8 cycloalkyl”, “C3-C8 cycloalkyl C1-C8 alkyl”, “C3-C8 cycloalkenyl”, “C3-C8 cycloalkenyl C1-C8 alkyl”, “heterocyclyl”, “heterocyclyl C1-C8 alkyl”, “aryl”, or “aryl C1-C8 alkyl” is each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₄, —SR₁₄, —(CO)OR₁₄, —(SO₂)R₁₄, —N(R₁₄)₂, and —O—(C1-C8 alkyl)-(CO)OR₁₄, or two adjacent carbon atoms on the ring form a fused ring with —OCH₂CH₂— or —OCH₂O— that is unsubstituted or substituted by halogen;

X₁₃ and X₁₄ each independently represent hydrogen, halogen, cyano, C1-C8 alkoxy, C1-C8 alkoxy C1-C8 alkyl, C1-C8 alkylcarbonyl, C1-C8 alkoxycarbonyl, C1-C8 alkylsulfonyl, C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, aryl, aryl C1-C8 alkyl, heterocyclyl, or heterocyclyl C1-C8 alkyl, or the group CX₁₃X₁₄ together forms a 5-8 membered carbocycle or oxygen-, sulfur-, or nitrogen-containing heterocycle, or the group NX₁₃X₁₄ together forms

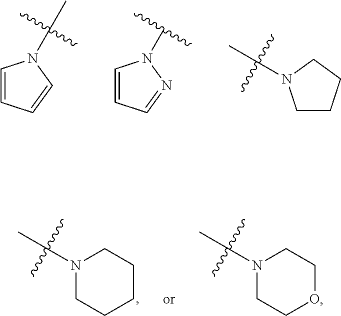

wherein, the “C1-C8 alkyl”, “C2-C8 alkenyl”, or “C2-C8 alkynyl” is each independently unsubstituted or substituted by halogen; the “C3-C8 cycloalkyl”, “C3-C8 cycloalkyl C1-C8 alkyl”, “C3-C8 cycloalkenyl”, “C3-C8 cycloalkenyl C1-C8 alkyl”, “aryl”, “aryl C1-C8 alkyl”, “heterocyclyl”, or “heterocyclyl C1-C8 alkyl” is each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₄, —SR₁₄, —(CO)OR₁₄, —(SO₂)R₁₄, —N(R₁₄)₂, and —O—(C1-C8 alkyl)-(CO)OR₁₄, or two adjacent carbon atoms on the ring form a fused ring with —OCH₂CH₂— or —OCH₂O— that is unsubstituted or substituted by halogen; the “5-8 membered carbocycle or oxygen-, sulfur-, or nitrogen-containing heterocycle” is unsubstituted or substituted by at least one group selected from C1-C8 alkyl, C1-C8 alkoxycarbonyl, and benzyl, or forms a fused ring structure with aryl or heterocyclyl; the

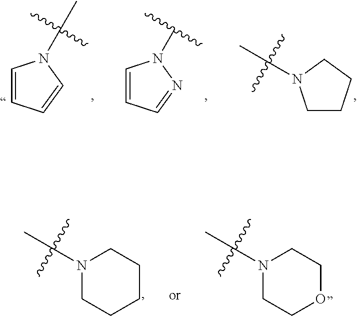 [SMILES: *n1cccc1 ; *n1cccn1 ; *N1CCCC1 ; *N1CCCCC1 ; *N1CCOCC1]

is unsubstituted or substituted by at least one group selected from oxo, C1-C8 alkyl, and C1-C8 alkoxycarbonyl.

In another specific embodiment, W₃ each independently represents hydrogen, C1-C6 alkyl, C2-C6 alkenyl, C2-C6 alkynyl, C3-C6 cycloalkyl, C3-C6 cycloalkenyl, heterocyclyl, aryl,

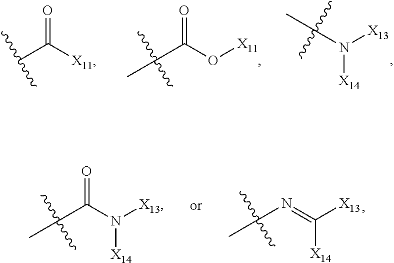

wherein, the “C1-C6 alkyl”, “C2-C6 alkenyl”, or “C2-C6 alkynyl” is each independently unsubstituted or substituted by 1, 2, or 3 groups selected from halogen, cyano, nitro, C3-C6 cycloalkyl, tri-C1-C6 alkylsilyl, C3-C6 cycloalkenyl, heterocyclyl, aryl,

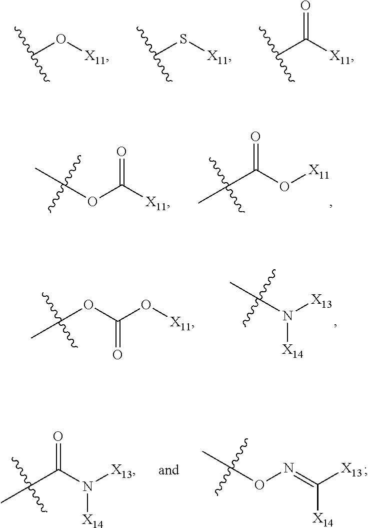

the “C3-C6 cycloalkyl”, “C3-C6 cycloalkenyl”, “heterocyclyl”, or “aryl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C6 alkyl)-(CO)OR₁₄, or two adjacent carbon atoms on the ring form a fused ring with —OCH₂CH₂— or —OCH₂O— that is unsubstituted or substituted by halogen;

W₄ and W₅ each independently represent hydrogen, C1-C6 alkyl, C2-C6 alkenyl, C2-C6 alkynyl, C3-C6 cycloalkyl, C3-C6 cycloalkenyl, heterocyclyl, aryl,

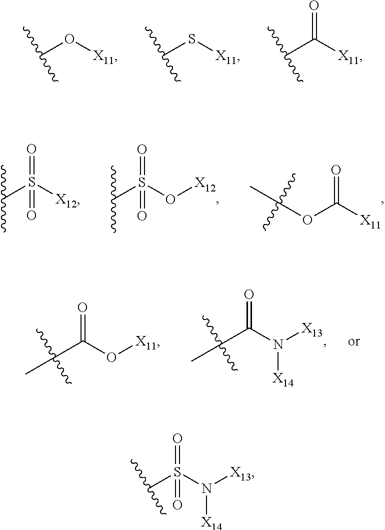

wherein, the “C1-C6 alkyl”, “C2-C6 alkenyl”, or “C2-C6 alkynyl” is each independently unsubstituted or substituted by 1, 2, or 3 groups selected from halogen, cyano, nitro, C3-C6 cycloalkyl, tri-C1-C6 alkylsilyl, C3-C6 cycloalkenyl, heterocyclyl, aryl,

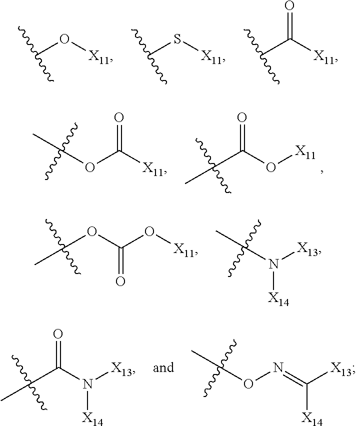

the “C3-C6 cycloalkyl”, “C3-C6 cycloalkenyl”, “heterocyclyl”, or “aryl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C6 alkyl)-(CO)OR₁₄, or two adjacent carbon atoms on the ring form a fused ring with —OCH₂CH₂— or —OCH₂O— that is unsubstituted or substituted by halogen;
or NW₄W₅ represents

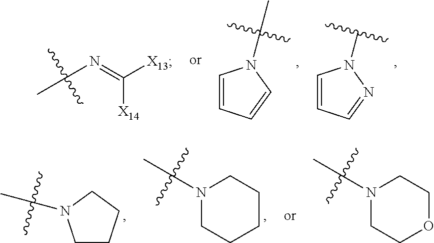

which is unsubstituted or substituted by 1, 2, or 3 groups selected from oxo, C1-C6 alkyl, and C1-C6 alkoxycarbonyl;

X₁₁ each independently represents hydrogen, C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, heterocyclyl, heterocyclyl C1-C6 alkyl, aryl, or aryl C1-C6 alkyl, wherein, the “C3-C6 cycloalkyl”, “C3-C6 cycloalkyl C1-C6 alkyl”, “C3-C6 cycloalkenyl”, “C3-C6 cycloalkenyl C1-C6 alkyl”, “heterocyclyl”, “heterocyclyl C1-C6 alkyl”, “aryl”, or “aryl C1-C6 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C6 alkyl)-(CO)OR₁₄, or two adjacent carbon atoms on the ring form a fused ring with —OCH₂CH₂— or —OCH₂O— that is unsubstituted or substituted by halogen;

X₁₂ each independently represents C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, heterocyclyl, heterocyclyl C1-C6 alkyl, aryl, or aryl C1-C6 alkyl, wherein, the “C3-C6 cycloalkyl”, “C3-C6 cycloalkyl C1-C6 alkyl”, “C3-C6 cycloalkenyl”, “C3-C6 cycloalkenyl C1-C6 alkyl”, “heterocyclyl”, “heterocyclyl C1-C6 alkyl”, “aryl”, or “aryl C1-C6 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C6 alkyl)-(CO)OR₁₄, or two adjacent carbon atoms on the ring form a fused ring with —OCH₂CH₂— or —OCH₂O— that is unsubstituted or substituted by halogen;

X₁₃ and X₁₄ each independently represent hydrogen, halogen, cyano, C1-C6 alkoxy, C1-C6 alkoxy C1-C6 alkyl, C1-C6 alkylcarbonyl, C1-C6 alkoxycarbonyl, C1-C6 alkylsulfonyl, C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, aryl, aryl C1-C6 alkyl, heterocyclyl, or heterocyclyl C1-C6 alkyl, or the group CX₁₃X₁₄ together forms a 5-8 membered saturated carbocycle,

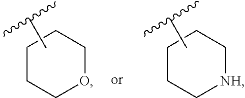

or the group NX₁₃X₁₄ together forms

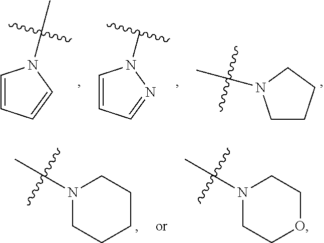

wherein, the “C1-C6 alkyl”, “C2-C6 alkenyl”, or “C2-C6 alkynyl” is each independently unsubstituted or substituted by halogen; the “C3-C6 cycloalkyl”, “C3-C6 cycloalkyl C1-C6 alkyl”, “C3-C6 cycloalkenyl”, “C3-C6 cycloalkenyl C1-C6 alkyl”, “aryl”, “aryl C1-C6 alkyl”, “heterocyclyl”, or “heterocyclyl C1-C6 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂ and —O—(C1-C6 alkyl)-(CO)OR₁₄, or two adjacent carbon atoms on the ring form a fused ring with —OCH₂CH₂— or —OCH₂O— that is unsubstituted or substituted by halogen; the “5-8 membered saturated carbocycle,

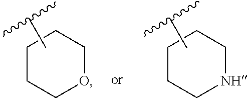

is unsubstituted or substituted by 1, 2 or 3 groups selected from C1-C6 alkyl, C1-C6 alkoxycarbonyl, and benzyl, or forms a fused ring structure with aryl or heterocyclyl; the

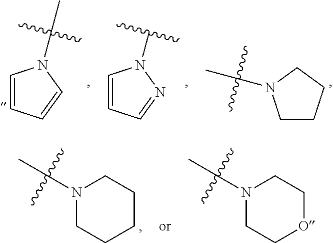

is unsubstituted or substituted by 1, 2, or 3 groups selected from oxo, C1-C6 alkyl, and C1-C6 alkoxycarbonyl.

In another specific embodiment, W₃ each independently represents hydrogen, C1-C6 alkyl, C2-C6 alkenyl, C2-C6 alkynyl, C3-C6 cycloalkyl, C3-C6 cycloalkenyl, heterocyclyl, aryl,

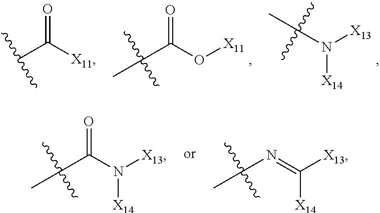

wherein, the “C1-C6 alkyl”, “C2-C6 alkenyl”, or “C2-C6 alkynyl” is each independently unsubstituted or substituted by 1, 2, or 3 groups selected from halogen, cyano, nitro, C3-C6 cycloalkyl, tri-C1-C6 alkylsilyl, C3-C6 cycloalkenyl, heterocyclyl, aryl,

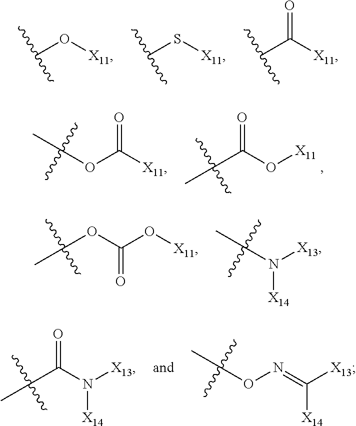

the “C3-C6 cycloalkyl”, “C3-C6 cycloalkenyl”, “heterocyclyl”, or “aryl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C3 alkyl)-(CO)OR₁₄, or two adjacent carbon atoms on the ring form a fused ring with —OCH₂CH₂— or —OCH₂O— that is unsubstituted or substituted by halogen;

W₄ and W₅ each independently represent hydrogen, C1-C6 alkyl, C2-C6 alkenyl, C2-C6 alkynyl, C3-C6 cycloalkyl, C3-C6 cycloalkenyl, heterocyclyl, aryl,

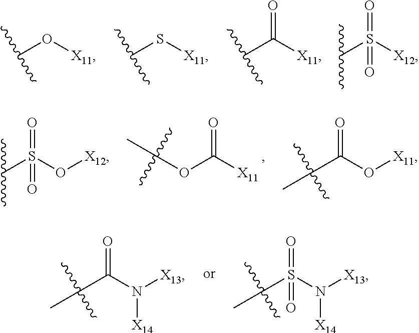

wherein, the “C1-C6 alkyl”, “C2-C6 alkenyl”, or “C2-C6 alkynyl” is each independently unsubstituted or substituted by 1, 2, or 3 groups selected from halogen, cyano, nitro, C3-C6 cycloalkyl, tri-C1-C6 alkylsilyl, C3-C6 cycloalkenyl, heterocyclyl, aryl,

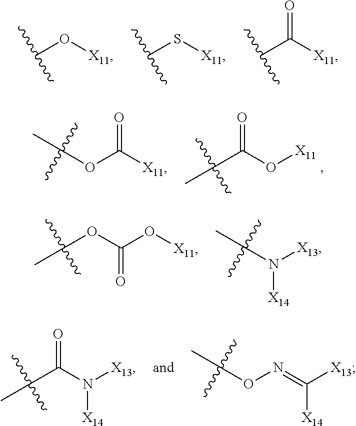

the “C3-C6 cycloalkyl”, “C3-C6 cycloalkenyl”, “heterocyclyl”, or “aryl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C3 alkyl)-(CO)OR₁₄, or two adjacent carbon atoms on the ring form a fused ring with —OCH₂CH₂— or —OCH₂O— that is unsubstituted or substituted by halogen;

or NW₄W₅ represents

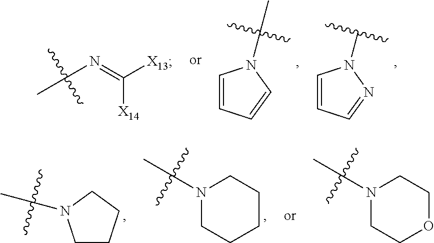

which is unsubstituted or substituted by 1, 2, or 3 groups selected from oxo, C1-C6 alkyl, and C1-C6 alkoxycarbonyl;

X₁₁ each independently represents hydrogen, C1-C6 alkyl, C2-C6 alkenyl, C2-C6 alkynyl, C3-C6 cycloalkyl, C3-C6 cycloalkyl C1-C3 alkyl, C3-C6 cycloalkenyl, C3-C6 cycloalkenyl C1-C3 alkyl, heterocyclyl, heterocyclyl C1-C3 alkyl, aryl, or aryl C1-C3 alkyl, wherein, the “C3-C6 cycloalkyl”, “C3-C6 cycloalkyl C1-C3 alkyl”, “C3-C6 cycloalkenyl”, “C3-C6 cycloalkenyl C1-C3 alkyl”, “heterocyclyl”, “heterocyclyl C1-C3 alkyl”, “aryl”, or “aryl C1-C3 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C3 alkyl)-(CO)OR₁₄, or two adjacent carbon atoms on the ring form a fused ring with —OCH₂CH₂— or —OCH₂O— that is unsubstituted or substituted by halogen;

X₁₂ each independently represents C1-C6 alkyl, C2-C6 alkenyl, C2-C6 alkynyl, C3-C6 cycloalkyl, C3-C6 cycloalkyl C1-C3 alkyl, C3-C6 cycloalkenyl, C3-C6 cycloalkenyl C1-C3 alkyl, heterocyclyl, heterocyclyl C1-C3 alkyl, aryl, or aryl C1-C3 alkyl, wherein, the “C3-C6 cycloalkyl”, “C3-C6 cycloalkyl C1-C3 alkyl”, “C3-C6 cycloalkenyl”, “C3-C6 cycloalkenyl C1-C3 alkyl”, “heterocyclyl”, “heterocyclyl C1-C3 alkyl”, “aryl”, or “aryl C1-C3 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C3 alkyl)-(CO)OR₁₄, or two adjacent carbon atoms on the ring form a fused ring with —OCH₂CH₂— or —OCH₂O— that is unsubstituted or substituted by halogen;

X₁₃ and X₁₄ each independently represent hydrogen, halogen, cyano, C1-C6 alkoxy, C1-C6 alkoxy C1-C3 alkyl, C1-C6 alkylcarbonyl, C1-C6 alkoxycarbonyl, C1-C6 alkylsulfonyl, C1-C6 alkyl, C2-C6 alkenyl, C2-C6 alkynyl, C3-C6 cycloalkyl, C3-C6 cycloalkyl C1-C3 alkyl, C3-C6 cycloalkenyl, C3-C6 cycloalkenyl C1-C3 alkyl, aryl, aryl C1-C3 alkyl, heterocyclyl, or heterocyclyl C1-C3 alkyl, or the group CX₁₃X₁₄ together forms a 5-8 membered saturated carbocycle,

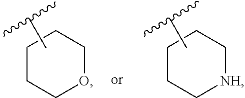

or the group NX₁₃X₁₄ together forms

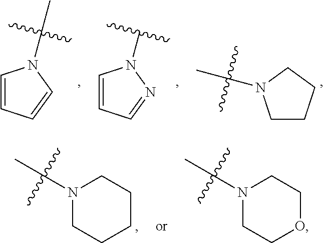

wherein, the “C1-C6 alkyl”, “C2-C6 alkenyl”, or “C2-C6 alkynyl” is each independently unsubstituted or substituted by halogen; the “C3-C6 cycloalkyl”, “C3-C6 cycloalkyl C1-C3 alkyl”, “C3-C6 cycloalkenyl”, “C3-C6 cycloalkenyl C1-C3 alkyl”, “aryl”, “aryl C1-C3 alkyl”, “heterocyclyl”, or “heterocyclyl C1-C3 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C3 alkyl)-(CO)OR₁₄, or two adjacent carbon atoms on the ring form a fused ring with —OCH₂CH₂— or —OCH₂O— that is unsubstituted or substituted by halogen; the “5-8 membered saturated carbocycle,

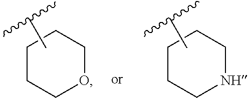

is unsubstituted or substituted by 1, 2, or 3 groups selected from C1-C6 alkyl, C1-C6 alkoxycarbonyl, and benzyl, or forms a fused ring with phenyl or thienyl; the

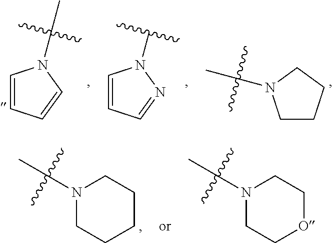

is unsubstituted or substituted by 1, 2, or 3 groups selected from oxo, C1-C6 alkyl, and C1-C6 alkoxycarbonyl.

In one specific embodiment, R₁₁ each independently represents C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, phenyl, or benzyl, wherein, the “C1-C8 alkyl”, “C2-C8 alkenyl”, or “C2-C8 alkynyl” is each independently unsubstituted or substituted by halogen; the “phenyl” or “benzyl” is each independently unsubstituted or substituted by at least one group selected from halogen, cyano, nitro, C1-C8 alkyl, halo C1-C8 alkyl, C1-C8 alkoxycarbonyl, C1-C8 alkylsulfanyl, C1-C8 alkylsulfonyl, C1-C8 alkoxy, and halo C1-C8 alkoxy;

R₁₂ each independently represents hydrogen, C1-C8 alkyl, C2-C8 alkenyl, C2-C8 alkynyl, C1-C8 alkoxy, C1-C8 alkylsulfonyl, C3-C8 cycloalkyl, C3-C8 cycloalkyl C1-C8 alkyl, C3-C8 cycloalkenyl, or C3-C8 cycloalkenyl C1-C8 alkyl, or the group N(R₁₂)₂ in —(CO)N(R₁₂)₂ or —(SO₂)N(R₁₂)₂ each independently represents

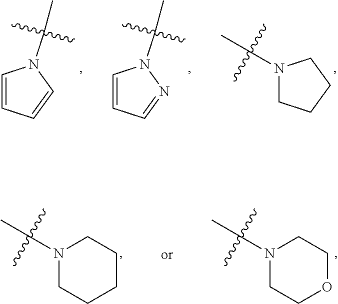 [SMILES: *n1cccc1 ; *n1cccn1 ; *N1CCCC1 ; *N1CCCCC1 ; *N1CCOCC1]

which is unsubstituted or substituted by at least one group selected from oxo, C1-C8 alkyl, and C1-C8 alkoxycarbonyl;

R₁₃ each independently represents C1-C8 alkyl, C2-C8 alkenyl, C2-C8 alkynyl, C3-C8 cycloalkyl, phenyl, or phenyl that is substituted by at least one group selected from halogen, cyano, nitro, C1-C8 alkyl, halo C1-C8 alkyl, C1-C8 alkoxy, halo C1-C8 alkoxy, C1-C8 alkoxycarbonyl, C1-C8 alkylsulfanyl, C1-C8 alkylsulfonyl, and phenoxy that is substituted by at least one group selected from halogen, cyano, nitro, C1-C8 alkyl, halo C1-C8 alkyl, C1-C8 alkoxy, and halo C1-C8 alkoxy;

R₁₄ each independently represents hydrogen, C1-C8 alkyl, halo C1-C8 alkyl, phenyl, or phenyl that is substituted by at least one group selected from halogen, cyano, nitro, C1-C8 alkyl, halo C1-C8 alkyl, C1-C8 alkoxycarbonyl, C1-C8 alkylsulfanyl, C1-C8 alkylsulfonyl, C1-C8 alkoxy, and halo C1-C8 alkoxy.

In one specific embodiment, R₁₁ each independently represents C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, phenyl, or benzyl, wherein, the “C1-C6 alkyl”, “C2-C6 alkenyl”, or “C2-C6 alkynyl” is each independently unsubstituted or substituted by halogen; the “phenyl” or “benzyl” is each independently unsubstituted or substituted by 1, 2, or 3 groups selected from halogen, cyano, nitro, C1-C6 alkyl, halo C1-C6 alkyl, C1-C6 alkoxycarbonyl, C1-C6 alkylsulfanyl, C1-C6 alkylsulfonyl, C1-C6 alkoxy, and halo C1-C6 alkoxy;

R₁₂ each independently represents hydrogen, C1-C6 alkyl, C2-C6 alkenyl, C2-C6 alkynyl, C1-C6 alkoxy, C1-C6 alkylsulfonyl, C3-C6 cycloalkyl, C3-C6 cycloalkyl C1-C6 alkyl, C3-C6 cycloalkenyl, or C3-C6 cycloalkenyl C1-C6 alkyl, or the group N(R₁₂)₂ in —(CO)N(R₁₂)₂ or —(SO₂)N(R₁₂)₂ each independently represents

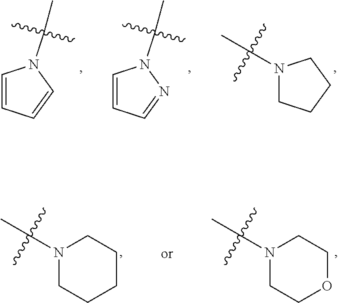 [SMILES: *n1cccc1 ; *n1cccn1 ; *N1CCCC1 ; *N1CCCCC1 ; *N1CCOCC1]

which is unsubstituted or substituted by 1, 2, or 3 groups selected from oxo, C1-C6 alkyl, and C1-C6 alkoxycarbonyl;

R₁₃ each independently represents C1-C6 alkyl, C2-C6 alkenyl, C2-C6 alkynyl, C3-C6 cycloalkyl, phenyl, or phenyl that is substituted by 1, 2, or 3 groups selected from halogen, cyano, nitro, C1-C6 alkyl, halo C1-C6 alkyl, C1-C6 alkoxy, halo C1-C6 alkoxy, C1-C6 alkoxycarbonyl, C1-C6 alkylsulfanyl, C1-C6 alkylsulfonyl, and phenoxy that is substituted by 1, 2, or 3 groups selected from halogen, cyano, nitro, C1-C6 alkyl, halo C1-C6 alkyl, C1-C6 alkoxy, and halo C1-C6 alkoxy;

R₁₄ each independently represents hydrogen, C1-C6 alkyl, halo C1-C6 alkyl, phenyl, or phenyl that is substituted by 1, 2 or 3 groups selected from halogen, cyano, nitro, C1-C6 alkyl, halo C1-C6 alkyl, C1-C6 alkoxycarbonyl, C1-C6 alkylsulfanyl, C1-C6 alkylsulfonyl, C1-C6 alkoxy, and halo C1-C6 alkoxy.

In another specific embodiment, R₁₁ each independently represents C1-C6 alkyl, C2-C6 alkenyl, C2-C6 alkynyl, C3-C6 cycloalkyl, C3-C6 cycloalkyl C1-C3 alkyl, C3-C6 cycloalkenyl, C3-C6 cycloalkenyl C1-C3 alkyl, phenyl, or benzyl, wherein, the “C1-C6 alkyl”, “C2-C6 alkenyl”, or “C2-C6 alkynyl” is each independently unsubstituted or substituted by halogen; the “phenyl” or “benzyl” is each independently unsubstituted or substituted by 1, 2, or 3 groups selected from halogen, cyano, nitro, C1-C6 alkyl, halo C1-C6 alkyl, C1-C6 alkoxycarbonyl, C1-C6 alkylsulfanyl, C1-C6 alkylsulfonyl, C1-C6 alkoxy, and halo C1-C6 alkoxy;

R₁₂ each independently represents hydrogen, C1-C6 alkyl, C2-C6 alkenyl, C2-C6 alkynyl, C1-C6 alkoxy, C1-C6 alkylsulfonyl, C3-C6 cycloalkyl, C3-C6 cycloalkyl C1-C3 alkyl, C3-C6 cycloalkenyl, or C3-C6 cycloalkenyl C1-C3 alkyl, or the group N(R₁₂)₂ in —(CO)N(R₁₂)₂ or —(SO₂)N(R₁₂)₂ each independently represents

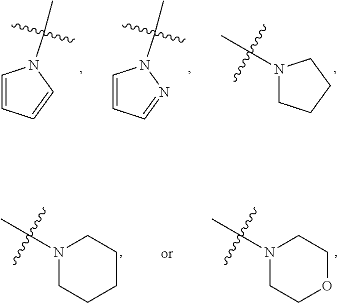 [SMILES: *n1cccc1 ; *n1cccn1 ; *N1CCCC1 ; *N1CCCCC1 ; *N1CCOCC1]

which is unsubstituted or substituted by 1, 2, or 3 groups selected from oxo, C1-C6 alkyl, and C1-C6 alkoxycarbonyl;

R₁₃ each independently represents C1-C6 alkyl, C2-C6 alkenyl, C2-C6 alkynyl, C3-C6 cycloalkyl, phenyl, or phenyl that is substituted by 1, 2, or 3 groups selected from halogen, cyano, nitro, C1-C6 alkyl, halo C1-C6 alkyl, C1-C6 alkoxy, halo C1-C6 alkoxy, C1-C6 alkoxycarbonyl, C1-C6 alkylsulfanyl, C1-C6 alkylsulfonyl, and phenoxy that is substituted by 1, 2, or 3 groups selected from halogen, cyano, nitro, C1-C6 alkyl, halo C1-C6 alkyl, C1-C6 alkoxy, and halo C1-C6 alkoxy;

R₁₄ each independently represents hydrogen, C1-C6 alkyl, halo C1-C6 alkyl, phenyl, or phenyl that is substituted by 1, 2, or 3 groups selected from halogen, cyano, nitro, C1-C6 alkyl, halo C1-C6 alkyl, C1-C6 alkoxycarbonyl, C1-C6 alkylsulfanyl, C1-C6 alkylsulfonyl, C1-C6 alkoxy, and halo C1-C6 alkoxy.

In the definitions of the compounds represented by the above-mentioned general formula and all of the structural formulas hereinafter, the technical terms used, whether used alone or used in compound words, represent the following substituent groups: an alkyl group which has more than two carbon atoms and may be linear or branched. For example, the alkyl in the compound word “-alkyl-(CO)OR₁₁” may be —CH₂—, —CH₂CH₂—, —CH(CH₃)—, —C(CH₃)₂—, etc. An alkyl group is, for example, C1 alkyl: methyl; C2 alkyl: ethyl; C3 alkyl: propyl such as n-propyl or isopropyl; C4 alkyl: butyl such as n-butyl, isobutyl, tert-butyl, or 2-butyl; C5 alkyl: pentyl such as n-pentyl; C6 alkyl: hexyl such as n-hexyl, isohexyl, and 1,3-dimethylbutyl. Similarly, alkenyl is, for example, vinyl, allyl, 1-methylprop-2-en-1-yl, 2-methylprop-2-en-1-yl, but-2-en-1-yl, but-3-en-1-yl, 1-methylbut-3-en-1-yl, and 1-methylbut-2-en-1-yl. Alkynyl is, for example, ethynyl, propargyl, but-2-yn-1-yl, but-3-yn-1-yl, or 1-methylbut-3-yn-1-yl. A multiple bond may be at any position of each unsaturated group. Cycloalkyl is a saturated carbocyclic ring system having for example three to six carbon atoms such as cyclopropyl, cyclobutyl, cyclopentyl, or cyclohexyl. Similarly, cycloalkenyl is monocyclic alkenyl having for example three to six carbon ring members such as cyclopropenyl, cyclobutenyl, cyclopentenyl, and cyclohexenyl, wherein a double bond can be at any position. A halogen is fluorine, chlorine, bromine, or iodine.

Unless otherwise specified, the “aryl” of the present invention includes, but is not limited to, phenyl, naphthyl,

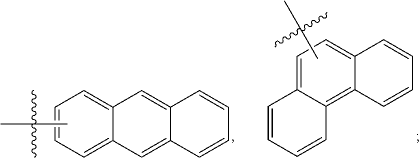

the “heterocyclyl” not only includes, but is not limited to, saturated or unsaturated non-aromatic cyclic groups,

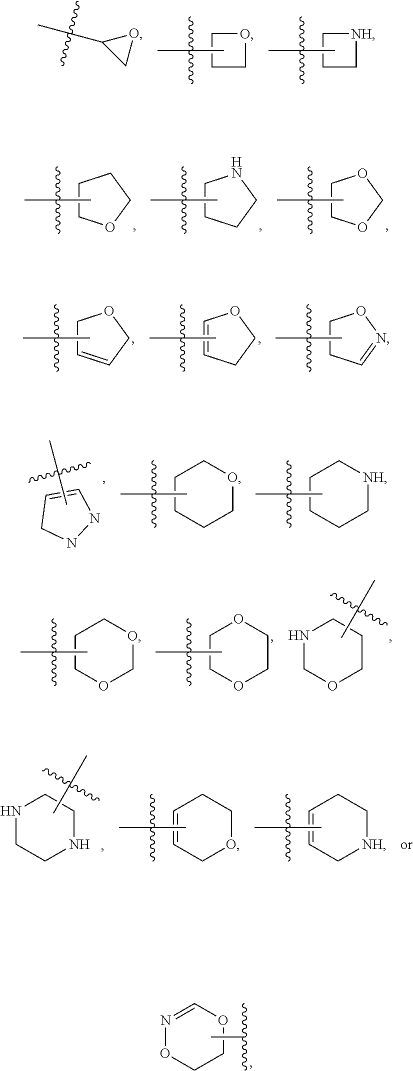

etc., but also includes, but is not limited to, heteroaryl, that is an aromatic cyclic group having for example 3 to 6 ring atoms and also optionally fused with a benzo ring. One to four (for example, 1, 2, 3, or 4) heteroatoms of the ring atoms are selected from oxygen, nitrogen, and sulfur, for example

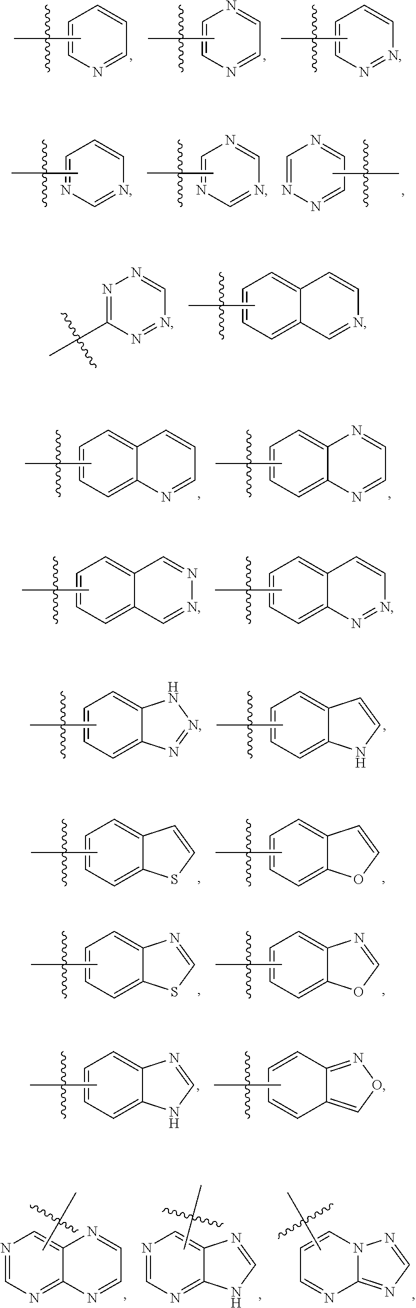

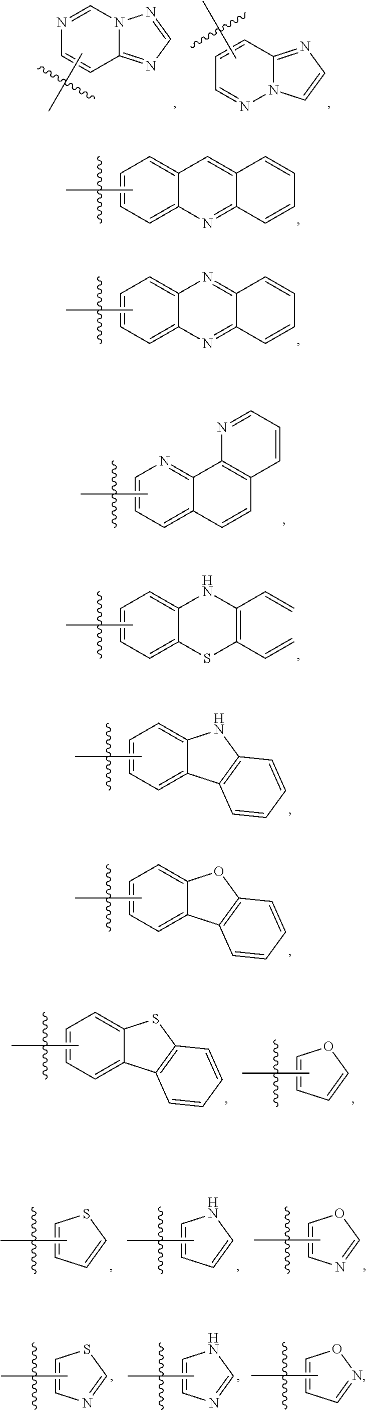

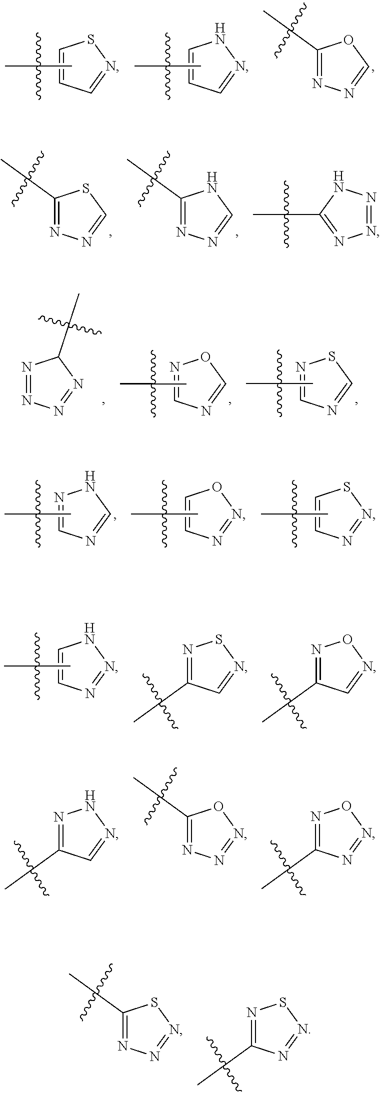

If one group is substituted by a group, this should be understood to mean that the group is substituted by one or more groups, which may be the same or different, selected from the mentioned groups. In addition, the same or different substitution characters contained in the same or different substituent groups are independently selected, which may be the same or different. This is also applicable to a ring system formed by different atoms and units. Meanwhile, the scope of the claims will exclude those compounds chemically unstable under standard conditions which are known to a person skilled in the art.

In addition, unless specifically defined, the term “substituted by at least one group” herein refers to being substituted by, for example, 1, 2, 3, 4, or 5 groups; a group (including heterocyclyl, aryl, etc.) without being specified a linking site may be linked at any position, including a site connected to C or N; if it is substituted, the substituent group may also substitute at any position as long as the valence bond theory is complied with. For example, the heteroaryl

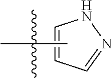

substituted by a methyl may represent

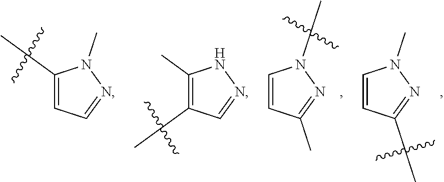

etc.

It should be pointed out that when the carbon atom (C*) connected to X₁ and X₂ in Formula I

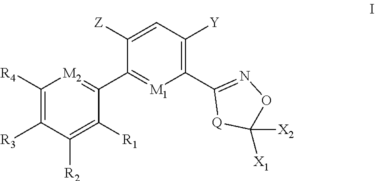

is a chiral center (i.e., when X₁ and X₂ are different), it is in R-configuration or S-configuration.

In the present invention, the stereochemical configuration at the position marked with * in Formula I is determined as the main (R) or (S) according to the Cahn-Ingold-Prelog system. However, the subject matter of the present invention also relates to all stereoisomers at other positions encompassed by Formula I, and mixtures thereof. Such compounds of Formula I contain, for example, one or more additional asymmetric carbon atoms or other double bonds that are not specified in Formula I. It is to be understood that the present invention comprises pure isomers and mixtures thereof of pure isomers enriched to varying degrees, wherein, the asymmetric carbon atom at the position marked with * is in R-configuration or S-configuration; or in the mixture, a compound or a compound of the same chemical structure has R-configuration or S-configuration at the position marked with *, or is present in such a proportion where the compound with R-configuration or S-configuration is predominantly present (at least 60% having R-configuration or S-configuration); meanwhile, other asymmetric carbon atoms may exist in racemic forms or may be resolved to varying degrees. As long as the stereochemical configuration at a position marked with * is eligible, possible stereoisomers defined by particular spatial forms such as enantiomers, diastereoisomers, Z- and E-isomers, are all encompassed by Formula I, and may be obtained from mixtures of stereoisomers using conventional methods, or may be prepared by stereoselective reactions in combination with the use of stereochemically pure starting materials.

The present invention also includes any keto-enol tautomeric form, a mixture and salt thereof, if various functional groups are present.

Stereoisomers are obtainable from the mixtures prepared by optical resolution. Stereoisomers may also be selectively prepared by employing stereoselective reactions and using optically active starting materials and/or auxiliaries. For optical resolution, conventional methods can often be employed (see Textbooks of Stereochemistry), such as those described below for resolving mixtures into diastereoisomers, for example, physical methods, e.g., crystallization, chromatography, particularly column chromatography and high pressure liquid chromatography, distillation methods carried out under reduced pressure as needed, extraction and other methods. Chromatographic separation on a chiral solid phase is usually adopted to separate residual mixtures of enantiomers. Suitable for a preparative amount or for use on an industrial scale are methods such as crystallization of diastereomeric salts, which can be obtained from compounds using optically active acids, and if acidic groups are present, optically active bases can be employed as needed.

A method for preparing the heterocycle-substituted aromatic compounds comprises the following steps:
subjecting the compound shown in the general formula II and the compound shown in the general formula III to reaction to prepare the compound as shown in the general formula I, and the chemical reaction equation is as follows:

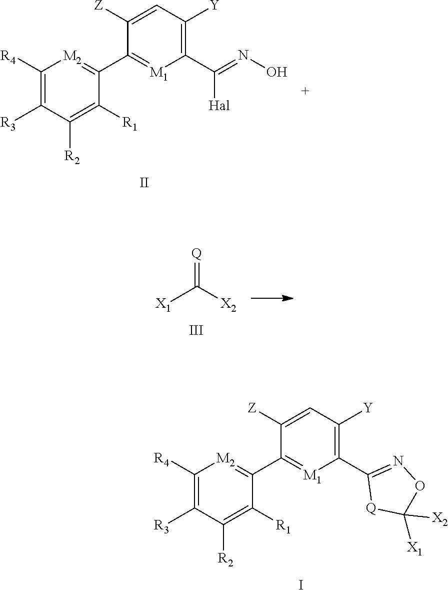

wherein, Hal represents halogen, preferably C1; the definitions of substituents X₁, X₂, R₁, R₂, R₃, R₄, M₁, M₂, Q, Y, and Z are as described above;
preferably, the reaction is carried out in the presence of a base and a solvent; more preferably, the base is selected from at least one of nitrogen-containing bases (e.g., C1-C6 alkylamine, preferably tri-(C1-C6)-alkylamine, e.g., triethylamine, trimethylamine, N-ethyldiisopropylamine);
the solvent is selected from at least one of aromatic hydrocarbons (e.g., benzene, chlorobenzene, toluene, cresol, or o-, n-, and p-xylene), THF, DMF, DMA, methanol, ethanol, acetonitrile, dichloroethane, DMSO, dioxane, dichloromethane, toluene, and ethyl acetate.

The compound as shown in the general formula II can be prepared with reference to the methods set forth in WO12130798, WO1404882, WO14048882, WO18228985, WO18228986, WO19034602, WO19145245, etc.

A herbicidal composition comprises a herbicidally effective amount of at least one of the heterocycle-substituted aromatic compounds, preferably, also comprising a formulation auxiliary.

A method for controlling weeds comprises applying a herbicidally effective amount of at least one of the heterocycle-substituted aromatic compounds or the herbicidal composition to a plant or a weedy area.

At least one of the heterocycle-substituted aromatic compounds or the herbicidal composition has use in controlling weeds; preferably, the heterocycle-substituted aromatic compound is used for preventing and controlling weeds among useful crops, and the useful crops are transgenic crops or crops treated by genome editing techniques.

The compounds of Formula I of the present invention have outstanding herbicidal activity against a broad spectrum of monocotyledonous and dicotyledonous harmful plants of economic importance. The active substances of the present invention also act effectively on perennial weeds which grow from root stocks, rhizomes, or other perennial organs and are difficult to control. In this context, it is generally immaterial whether the substances are applied before sowing, before emergence, or after emergence. Representative examples of monocotyledonous and dicotyledonous weed florae which can be controlled by the compound of the present invention may be specifically mentioned, without limiting to certain species. Examples of weed species on which the active substances act efficiently include the monocotyledons such as annual Avena, Lolium, Alopecurus, Phalaris, Echinochloa, Digitaria, Setaria and Cyperus, as well as perennial Agropyron, Cynodon, Imperata and Sorghum, and perennial Cyperus.

In the case of dicotyledonous weed species, the spectrum of action extends to species such as annual Galium, Viola, Veronica, Lamium, Stellaria, Amaranthus, Sinapis, Ipomoea, Sida, Matricaria and Abutilon, as well as perennial weeds Convolvulus, Cirsium, Rumex, and Artemisia. The active substances of the present invention perform outstanding control of harmful plants such as Echinochloa, Sagittaria, Alisma, Eleocharis, Scirpus and Cyperus under the specific condition of paddy rice growing. If the compounds of the present invention are applied to soil surface prior to germination, the weed seedlings are either prevented completely from emerging, or the weeds stop growing when reaching the cotyledon stage and eventually die completely after three to four weeks. In particular, the compounds of the present invention exhibit excellent activity against Apera spica venti, Matsumurella chinense, Fallopia convolvulus, Stellaria media, Veronica hederifolia, Veronica persica, Viola tricolor, Amaranthus, Galium, and Kochia.

Although the compounds of the present invention have excellent herbicidal activity against monocotyledonous and dicotyledonous weeds, there is no damage at all to crop plants of economic importance such as wheat, barley, rye, rice, maize, sugarbeet, cotton and soybean, or the damage is negligible. In particular, the compounds have excellent compatibility with cereals such as wheat, barley, and maize, in particular wheat. Therefore, the compounds of the present invention are highly suitable for selectively controlling undesired plants in plantings for agricultural or ornamental use.

Owing to their herbicidal properties, these active substances may be employed for controlling harmful plants in plantings of genetically engineered plants that are known or to be introduced. Transgenic plants usually have advantageous traits, for example, resistance to certain pesticides, in particular to certain herbicides; resistance to plant diseases or pathogenic microorganisms of plant diseases such as certain insects or microorganisms including fungi, bacteria, or viruses. Other particular traits relate to the following conditions of the product, for example, quantity, quality, storage stability, composition, and special ingredients. Thus, it is known that the obtained transgenic plant product has an increased starch content, or modified starch quality, or a different fatty acid composition.

The compounds of Formula I of the present invention or salts thereof are preferably used in plantings of transgenic crops and ornamental plants of economic importance, for example, cereals such as wheat, barley, rye, oats, millet, rice, manioc, and maize; or in plantings of vegetable plants such as sugarbeet, cotton, soybean, rapeseed, potato, tomato, pea and the like. The compounds of Formula I are preferably used as herbicides in plantings of useful plants which are resistant, or have been made resistant by genetic engineering, against the toxic effects of the herbicides.

Conventional ways for breeding plants which have modified traits compared to known plants include, for example, conventional breeding methods and breeding of mutant strains. In other words, novel plants having improved traits may be generated with the aid of genetic engineering methods (see, e.g., EP-0221044 A, EP-0131624 A). For example, several methods have been described:
changing crop plants using genetic engineering to modify the starch synthesis in plants (e.g., WO 92/11376, WO 92/14827, WO 91/19806);
transgenic crop plants resistant to certain herbicides, including glufosinate herbicides (e.g., EP-0242236 A, EP-0242246 A), or glyphosate herbicides (WO 92/00377), or sulfonylurea herbicides (EP-0257993 A, U.S. Pat. No. 5,013,659 A);
transgenic crop plants, for example cotton, able to produce Bacillus thuringiensis toxins (Bt toxins) which impart resistance to certain pests that invade the plants (EP-0142924 A, EP-0193259 A);
transgenic crop plants having a modified fatty acid composition (WO 91/13972).

Numerous molecular biology techniques which allow the preparation of transgenic plants having modified traits are known (see, e.g., Sambrook et al., 1989, Molecular Cloning, A Laboratory Manual, 2^{nd} edition, Cold Spring Harbor Laboratory Press, Cold Spring Harbor, N.Y.; or Winnacker, “Gene und Klone” [Genes and Clones], V C H Weinheim, 2^{nd} edition, 1996; or Christou, “Trends in Plant Science”, 1 (1996) 423-431). In order to carry out genetic engineering manipulations, it is possible to introduce a nucleic acid molecule into a plasmid, allowing mutagenesis or a sequence change to occur by recombination of the DNA sequence. Using the above-mentioned standard processes, it is possible to, for example, substitute bases, remove partial sequences, or add natural or synthetic sequences. To link DNA fragments with each other, it is possible to attach adaptors or linkers to the fragments.

A plant cell having a gene product of reduced activity may be prepared by the following methods, for example, by expressing at least one appropriate antisense-RNA and sense-RNA to achieve a cosuppression effect, or by expressing at least one appropriately constructed ribozyme which specifically cleaves the transcripts of the above-mentioned gene product.

To this end, it is possible to employ a DNA molecule which comprises all the coding sequences of the gene product including any flanking sequence that may be present and a DNA molecule which comprises only parts of the coding sequences that must be long enough to cause an antisense effect in the cells. It is also possible to use sequences which have a high degree of homology but are not entirely identical to the coding sequences of the gene product.

When nucleic acid molecules are expressed in a plant, the synthesized protein may be localized in any desired compartment of the plant cell. However, to achieve localization in a certain compartment, it is possible, for example, to link the coding region with the DNA sequences to ensure localization in the certain location. Such sequences are known to a person skilled in the art (see, e.g., Braun et al., EMBO J., 11 (1992), 3219-3227; Wolter et al., Proc. Natl. Acad. Sci. USA, 85 (1988), 846-850; Sonnewald et al., Plant J., 1 (1991), 95-106).

The transgenic plant cells can be recombined onto a whole plant using known techniques. A transgenic plant may be of any desired plant species, i.e., a monocotyledonous and dicotyledonous plant. In this manner, it is possible to obtain transgenic plants having modified traits by overexpression, suppression, or inhibition of homologous (=natural) genes or gene sequences, or by expression of heterologous (=foreign) genes or gene sequences.

When using the active substances of the present invention in a transgenic crop, in addition to the inhibitory effect against harmful plants which can be observed in other crops, there are frequently special effects on the corresponding transgenic crop, for example, improving or broadening the spectrum of weeds which can be controlled, modifying the application amount during application, excellent combination of the drug resistance of the preferred transgenic crop and herbicide performance, as well as effects on the growth and the yield of the transgenic crop plant. The present invention therefore also provides use of the compounds as herbicides for controlling harmful plants among transgenic crop plants.

In addition, the compounds of the present invention are able to significantly regulate the growth of a crop plant. These compounds are employed for the targeted control of plant constituents and for facilitating harvesting by engaging in plant metabolism in a regulating manner, for example by provoking desiccation and stunted growth. Furthermore, they are also suitable for regulating and inhibiting undesirable plant growth without destroying the growth of crop plants. Inhibition of plant growth plays a key role in many monocotyledonous and dicotyledonous crops as it can reduce or completely prevent lodging.

The compounds of the present invention may be applied in customary formulations in the form of wettable powders, emulsifiable concentrates, sprayable solutions, dusts, or granules. The present invention therefore also provides herbicidal compositions comprising the compounds of Formula I. The compounds of Formula I may be formulated in numerous ways depending on the prevailing physical parameters in biology and/or chemistry. Examples of suitable formulation options are: wettable powders (WP), water-soluble powders (SP), water-soluble concentrates, emulsifiable concentrates (EC), emulsions such as oil-in-water and water-in-oil emulsions (EW), sprayable solutions, suspension concentrates (SC), oil dispersions (OD), oil- or water-based dispersions, oil-miscible solutions, dust powders (DP), capsule suspensions (CS), seed-dressing compositions, granules for broadcasting and soil application, spray granules, coating granules and adsorption granules, water-dispersible granules (WG), water-soluble granules (SG), ULV (Ultra-low volume) formulations, microcapsules, and waxes. These individual formulation types are known and described in the following literature, for example, Winnacker-Küchler, “Chemische Technologie” [Chemical Technology], Volume 7, C. Hauser Verlag Munich, 4^{th} edition, 1986; Wade van Valkenburg, “Pesticide Formulations”, Marcel Dekker, N.Y, 1973; K. Martens, “Spray Drying” Handbook, 3^{rd} edition, 1979, G. Goodwin Ltd. London.

Necessary formulation auxiliaries such as inert materials, surfactants, solvents and other additives are likewise known and described in the following documents, for example, Watkins, “Handbook of Insecticide Dust Diluents and Carriers”, 2^{nd} edition, Dorland Books, Caldwell N.J.; H. v. Olphen, “An Introduction to Clay Colloid Chemistry”, 2^{nd} edition, J. Wiley & Sons, N.Y.; C. Marsden, “Solvents Guide”, 2^{nd} edition, Interscience, N.Y. 1963; “McCutcheon's Detergents and Emulsifiers Annual”, MC Publ. Corp., Ridgewood N.J.; Sisley and Wood, “Encyclopedia of Surface Active Agents”, Chem. Publ. Co. Inc., N.Y., 1964; Schönfeldt, “Grenzflüchenaktive Äthylenoxidaddkte” [Surface-active ethylene oxide adducts], Wiss. Verlagagesell, Stuttgart, 1976; Winnacker-Küchler, “Chemische Technologie” [Chemical Technology], Volume 7, C. Hauser Verlag Munich, 4^{th} edition, 1986.

Wettable powders are uniformly dispersible in water which contain, in addition to the active substance, a diluent or inert substance, ionic and/or nonionic surfactant (wetting agent, dispersant), for example polyethoxylated alkyl phenol, polyethoxylated fatty alcohol, polyethoxylated fatty amine, fatty alcohol polyglycol ether sulfate, alkane sulfonate, alkylphenyl sulfonate, sodium lignosulfonate, sodium 2,2′-dinaphthylmethane-6,6′-disulfonates, sodium dibutyl naphthalenesulfonate, or sodium methyl oleoyl taurate. To prepare the wettable powders, the herbicidally active substance is finely ground, for example, in a customary apparatus such as hammer mill, fan mill, and air-jet mill, and mixed with formulation auxiliaries simultaneously or sequentially.

Emulsifiable concentrates are prepared by dissolving the active substance in an organic solvent, for example butanol, cyclohexanone, dimethylformamide, xylene or aromatic compounds with relatively high boiling point or hydrocarbons or mixtures of the solvents, with the addition of one or more ionic and/or nonionic surfactants (emulsifiers). Examples of emulsifiers which can be used are calcium alkylarylsulfonates such as calcium dodecylbenzene sulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters such as sorbitan fatty acid esters, or polyoxyethylene sorbitan esters such as polyoxyethylene sorbitan fatty acid esters.

Dusts are obtained by grinding the active substance with finely-divided solid substances, for example talc, natural clays such as kaolin, bentonite and pyrophyllite, or diatomaceous earth. Water- or oil-based suspensions may be prepared by the following methods, for example, by wet milling using a commercially customary bead mill, with or without the addition of the surfactants mentioned above in the case of another formulation type.

Emulsions such as oil-in-water emulsions (EW) may be prepared by means of stirrers, colloid mills and/or static mixers using aqueous organic solvents and, if desired, the surfactants as mentioned above in the case of another formulation type may be added.

Granules may be prepared by the following methods that either spraying the active substance onto the adsorptive and granulating with inert materials, or concentrating the active substance onto the surface of carriers such as sand and kaolinite and granulating inert materials by means of adhesive binders such polyvinyl alcohol, sodium polyacrylate, or mineral oils. Suitable active substances may also be granulated in the manner which is customary for the preparation of fertilizer granules. If desired, fertilizers may be mixed in. Water-dispersible granules are prepared by customary methods such as spray-drying, fluidized-bed granulation, disk granulation, or mixing using high-speed mixers and extrusion without solid inert materials.

For the preparation methods of granules using disk, fluidized-bed, extruder and spray, see the following processes in, for example, “Spray-Drying Handbook” 3^{rd} edition, 1979, G. Goodwin Ltd., London; J. E. Browning, “Agglomeration”, Chemical and Engineering, 1967, pages 147 ff.; “Perry's Chemical Engineer's Handbook”, 5^{th} edition, McGraw-Hill, New York 1973, pp. 8-57. For further details on the formulation of crop protection products, see, for example G. C. Klingman, “Weed Control as a Science”, John Wiley and Sons Inc., New York, 1961, pages 81-96 and J. D. Freyer, S. A. Evans, “Weed Control Handbook”, 5^{th} edition, Blackwell Scientific Publications, Oxford, 1968, pages 101-103.

An agrochemical formulation generally contains from 0.1 to 99% by weight, in particular from 0.1 to 95%, of the active substance of Formula I. In a wettable powder, the concentration of the active substance is, for example, from about 10 to 99% by weight, and the remainder to 100% by weight consists of customary formulation constituents. In an emulsifiable concentrate, the concentration of the active substance may be from about 1 to 90%, preferably from 5 to 80%, by weight. A dust formulation contains from 1 to 30% by weight of the active substance, preferably from 5 to 20% by weight of the active substance in usual cases, while a sprayable solution contains from about 0.05 to 80%, preferably from 2 to 50%, by weight of the active substance. In the case of water-dispersible granules, the content of the active substance mainly depends on whether the active substance is liquid or solid and on auxiliaries, fillers, etc. that are used during granulation. In water-dispersible granules, the content of the active substance, for example, is between 1 and 95% by weight, preferably between 10 and 80% by weight.

In addition, formulations of the active substances may include tackifiers, wetting agents, dispersants, emulsifiers, penetrants, preservatives, antifreeze agents, solvents, fillers, carriers, colorants, defoamers, evaporation suppressors as well as pH and viscosity modifiers which are usually customary in all cases.

Based on these formulations, it is also possible to produce mixtures with other pesticidally active substances, for example insecticides, acaricides, herbicides and fungicides, and also with safeners, fertilizers and/or plant growth regulators, in a manner of pre-mix or tank mix.

In mixed formulations or tank-mix formulations, suitable active substances which can be mixed with the active substances of the present invention are the known substances described, for example in World Herbicide New Product Technology Handbook, China Agricultural Science and Farming Techniques Press, 2010. 9 and in the literature cited herein. For example, the herbicidally active substances mentioned below may be mixed with the compounds of Formula I (noted that the compounds are either named by the “common name” in accordance with the International Organization for Standardization (ISO) or by the chemical names, accompanied with a customary code number if appropriate): acetochlor, butachlor, alachlor, propisochlor, metolachlor, S-metolachlor, pretilachlor, propachlor, ethachlor, napropamide, napropamide-M, propanil, mefenacet, diphenamid, diflufenican, ethaprochlor, beflubutamid, bromobutide, dimethenamid, dimethenamid-P, etobenzanid, flufenacet, thenylchlor, metazachlor, isoxaben, flamprop-M-methyl, flamprop-M-propyl, allidochlor, pethoxamid, chloranocryl, cypromid, mefluidide, monalide, delachlor, prynachlor, terbuchlor, xylachlor, dimethachlor, cisanilide, trimexachlor, clomeprop, propyzamide, pentanochlor, carbetamide, benzoylprop-ethyl, cyprazole, butenachlor, tebutam, benzipram, quinonamid, dichlofluanid, naproanilide, diethatyl-ethyl, naptalam, flufenacet, EL-177, benzadox, chlorthiamid, chlorophthalimide, isocarbamide, picolinafen, atrazine, simazine, prometryn, cyanatryn, simetryn, ametryn, propazine, dipropetryn, SSH-108, terbutryn, terbuthylazine, triaziflam, cyprazine, proglinazine, trietazine, prometon, simeton, aziprotryne, desmetryn, dimethametryn, procyazine, mesoprazine, sebuthylazine, secbumeton, terbumeton, methoprotryne, cyanatryn, ipazine, chlorazine, atraton, pendimethalin, eglinazine-ethyl, cyanuric acid, indaziflam, chlorsulfuron, metsulfuron-methyl, bensulfuron-methyl, chlorimuron-ethyl, tribenuron-methyl, thifensulfuron-methyl, pyrazosulfuron-ethyl, mesosulfuron, iodosulfuron-methyl sodium, foramsulfuron, cinosulfuron, triasulfuron, sulfometuron methyl, nicosulfuron, ethametsulfuron-methyl, amidosulfuron, ethoxysulfuron, cyclosulfamuron, rimsulfuron, azimsulfuron, flazasulfuron, monosulfuron, monosulfuron-ester, flucarbazone-sodium, flupyrsulfuron-methyl, halosulfuron-methyl, oxasulfuron, imazosulfuron, primisulfuron, propoxycarbazone, prosulfuron, sulfosulfuron, trifloxysulfuron, triflusulfuron-methyl, tritosulfuron, sodium metsulfuron methyl, flucetosulfuron, HNPC-C9908, orthosulfamuron, propyrisulfuron, metazosulfuron, acifluorfen, fomesafen, lactofen, fluoroglycofen, oxyfluorfen, chlornitrofen, aclonifen, ethoxyfen-ethyl, bifenox, nitrofluorfen, chlomethoxyfen, fluorodifen, fluoronitrofen, furyloxyfen, nitrofen, TOPE, DMNP, PPG1013, AKH-7088, halosafen, chlortoluron, isoproturon, linuron, diuron, dymron, fluometuron, benzthiazuron, methabenzthiazuron, cumyluron, ethidimuron, isouron, tebuthiuron, buturon, chlorbromuron, methyldymron, phenobenzuron, SK-85, metobromuron, metoxuron, monolinuron, monuron, siduron, fenuron, fluothiuron, neburon, chloroxuron, noruron, isonoruron, cycluron, thiazfluron, tebuthiuron, difenoxuron, parafluron, methylamine tribunil, karbutilate, trimeturon, dimefuron, monisouron, anisuron, methiuron, chloreturon, tetrafluron, phenmedipham, phenmedipham-ethyl, desmedipham, asulam, terbucarb, barban, propham, chlorpropham, rowmate, swep, chlorbufam, carboxazole, chlorprocarb, fenasulam, BCPC, CPPC, carbasulam, butylate, benthiocarb, vernolate, molinate, triallate, dimepiperate, esprocarb, pyributicarb, cycloate, di-allate, EPTC, ethiolate, orbencarb, pebulate, prosulfocarb, tiocarbazil, CDEC, dimexano, isopolinate, methiobencarb, 2,4-D-butyl, MCPA-sodium, 2,4-D-isooctyl, MCPA-isooctyl, 2,4-D-sodium, 2,4-D-dimethylammonium, MCPA-thioethyl, MCPA, dichlorprop, salts of dichlorprop-P, 2,4-DB, mecoprop, salts of mecoprop, MCPB, 2,4,5-T, fenoprop, 2,4,5-TB, MCPA-ammonium, dicamba, erbon, chlorfenac, disul, 2,3,6-TBA, chloramben, methoxy-2,3,6-TBA, diclofop-methyl, fluazifop-butyl, fluazifop-P-butyl, haloxyfop-methyl, haloxyfop-P, quizalofop-ethyl, quizalofop-P-ethyl, fenoxaprop-ethyl, fenoxaprop-P-ethyl, propaquizafop, cyhalofop-butyl, metamifop, clodinafop-propargyl, fenthiaprop-ethyl, chlorazifop-propargyl, poppenate-methyl, trifopsime, isoxapyrifop, paraquat, diquat, oryzalin, ethalfluralin, isopropalin, nitralin, profluralin, prodinamine, benfluralin, fluchloraline, dinitramina, dipropalin, chlornidine, methalpropalin, dinoprop, glyphosate, anilofos, glufosinate ammonium, amiprophos-methyl, sulphosate, piperophos, bialaphos, bensulide, butamifos, fosamine-ammonium, 2,4-DEP, H-9201, DMPA, imazapyr, imazethapyr, imazaquin, imazamox, imazamox-ammonium, imazapic, imazamethabenz-methyl, fluroxypyr, fluroxypyr-isooctyl ester, clopyralid, picloram, trichlopyr, dithiopyr, haloxydine, pyriclor, thiazopyr, fluridone, aminopyralid, diflufenzopyr, triclopyr-butotyl, cliodinate, sethoxydim, clethodim, cycloxydim, alloxydim, profoxydim, butroxydim, tralkoxydim, tepraloxydim, buthidazole, metribuzin, hexazinone, metamitron, ethiozin, ametridione, amibuzin, bromoxynil, bromoxynil octanoate, ioxynil octanoate, ioxynil, dichlobenil, diphenylacetonitrile, pyraclonil, chloroxynil, iodobonil, flumetsulam, florasulam, penoxsulam, metosulam, cloransulam-methyl, diclosulam, pyroxsulam, benzofluor, bispyribac, pyribenzoxim, pyriftalid, pyriminobac-methyl, pyrithiobac-sodium, benzobicylon, mesotrione, sulcotrione, tembotrione, tefuryltrione, bicyclopyrone, ketospiradox, isoxaflutole, isoxachlortole, fenoxasulfone, methiozolin, fluazolate, pyraflufen-ethyl, pyrazolynate, difenzoquat, pyrazoxyfen, benzofenap, nipyraclofen, pyrasulfotole, topramezone, pyroxasulfone, cafenstrole, flupoxam, amitrole, amicarbazone, azafenidin, carfentrazone, sulfentrazone, bencarbazone, benzfendizone, butafenacil, bromacil, isocil, lenacil, terbacil, flupropacil, cinidon-ethyl, flumiclorac, flumioxazin, flumipropyn, MK-129, flumezin, (sodium)pentachlorophenol, dinoseb, dinoterb, dinoterb acetate, dinosam, DNOC, chloronitrophene, medinoterb acetate, dinofenate, oxadiargyl, oxadiazon, pentoxazone, profluazol, fluthiacet-methyl, fentrazamide, flufenpyr-ethyl, pyrazon, brompyrazon, metflurazon, kusakira, dimidazon, oxapyrazon, norflurazon, pyridafol, quinclorac, quinmerac, bentazone, pyridate, oxaziclomefone, benazolin-ethyl, clomazone, cinmethylin, pyribambenz-isopropyl, pyribambenz-propyl, indanofan, sodium chlorate, dalapon, trichloroacetic acid, monochloroacetic acid, hexachloroacetone, flupropanate, cyperquat, bromofenoxim, epronaz, methazole, flurtamone, benfuresate, ethofumesate, tioclorim, chlorthal, fluorochloridone, tavron, acrolein, bentranil, tridiphane, chlorfenprop-methyl, thidiazimin, phenisopham, busoxinone, methoxyphenone, saflufenacil, clacyfos, chloropon, alorac, diethamquat, etnipromid, iprymidam, ipfencarbazone, thiencarbazone-methyl, pyrimisulfan, chlorflurazole, tripropindan, sulglycapin, prosulfalin, cambendichlor, aminocyclopyrachlor, rodethanil, benoxacor, fenclorim, flurazole, fenchlorazole, cloquintocet-mexyl, oxabetrinil, MG191, cyometrinil, DKA-24, mefenpyr-diethyl, furilazole, fluxofenim, isoxadifen-ethyl, dichlormid, halauxifen-methyl, DOW848, UBH-509, D489, LS 82-556, KPP-300, NC-324, NC-330, KH-218, DPX-N8189, SC-0744, DOWC0535, DK-8910, V-53482, PP-600, MBH-001, KIH-9201, ET751, KIH-6127, and KIH-2023.

For use, the commercially available formulations are, if needed, diluted in customary manners, for example diluted with water in the cases of wettable powders, emulsifiable concentrates, dispersions, and water-dispersible granules. For products in the form of dusts, granules for soil application, or solutions for broadcasting and spray, usually no further dilution with inert materials is required prior to use. The required application amount of the compounds of Formula I varies with the external conditions such as temperature, humidity, the nature of the used herbicide and the like. It may have a large variation range, for example, between 0.001 and 1.0 kg a.i./ha, or more active substances, but preferably between 0.005 and 750 g a.i./ha, in particular between 0.005 and 250 g a.i./ha.

### DETAILED EMBODIMENTS OF THE INVENTION

The following examples are used to illustrate the present invention and should not be construed as limiting the present invention in any way. The scope of rights claimed by the present invention is described in the Claims.

In view of the economy and diversity of the compounds, some compounds were preferably synthesized. Among the many synthesized compounds, selected ones are listed in Table 1 below. The specific compound structures and corresponding compound information are as set forth in Tables 1-2. The compounds in Table 1 are only to better illustrate the present invention but do not limit the present invention. For a person skilled in the art, it should not be understood that the scope of the above-mentioned subject matters of the present invention is limited to the following compounds.

TABLE 1
Compound structure

| | | | | | | | | | | | I |
|---|---|---|---|---|---|---|---|---|---|---|---|
| 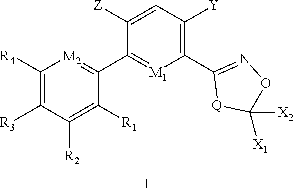 | | | | | | | | | | | |
| No. | X₁ | X₂ | Y | Z | Q | M₁ | M₂ | R₁ | R₂ | R₃ | R₄ |
| 1 | CH₃ | 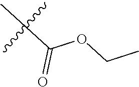 [SMILES: *C(=O)OCC] | Cl | F | CH₂ | CH | N | Cl | CH₃ | CF₃ | H |
| 2 | CH₃ | 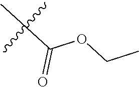 [SMILES: *C(=O)OCC] | Cl | F | CH₂ | N | CH | Cl | H | CF₃ | H |
| 3 | CH₃ | 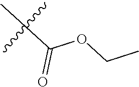 [SMILES: *C(=O)OCC] | Cl | F | CH₂ | N | N | Cl | H | CF₃ | H |
| 4 | CH₃ | 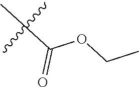 [SMILES: *C(=O)OCC] | Cl | F | CH₂ | N | N⁺—O⁻ | Cl | H | CF₃ | H |
| 5 | CH₃ | 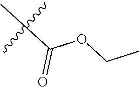 [SMILES: *C(=O)OCC] | Cl | OH | CH₂ | CH | N | Cl | H | CF₃ | H |
| 6 | CH₃ | 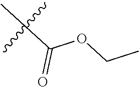 [SMILES: *C(=O)OCC] | CN | H | CH₂ | CH | N | Cl | H | CF₃ | H |
| 7 | CH₃ | 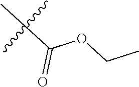 [SMILES: *C(=O)OCC] | NO₂ | F | CH₂ | CH | N | Cl | H | CF₃ | H |
| 8 | CH₃ | 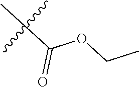 [SMILES: *C(=O)OCC] | NH₂ | F | CH₂ | CH | N | Cl | H | CF₃ | H |
| 9 | CH₃ | 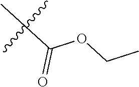 [SMILES: *C(=O)OCC] | CF₃ | F | CH₂ | CH | N | Cl | H | CF₃ | H |
| 10 | CH₃ | 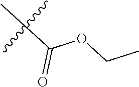 [SMILES: *C(=O)OCC] | Br | F | CH₂ | CH | N | Cl | H | CF₃ | H |
| 11 | CH₃ | 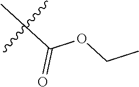 [SMILES: *C(=O)OCC] | CN | F | CH₂ | CH | N | Cl | H | CF₃ | H |
| 12 | 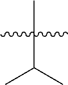 [SMILES: *C(C)C] | 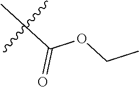 [SMILES: *C(=O)OCC] | Cl | F | CH₂ | CH | N | Cl | H | CF₃ | H |
| 13 | 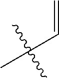 [SMILES: *C=C] | 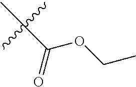 [SMILES: *C(=O)OCC] | Cl | F | CH₂ | CH | N | Cl | H | CF₃ | H |
| 14 | 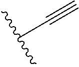 [SMILES: *C#C] | 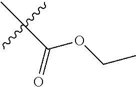 [SMILES: *C(=O)OCC] | Cl | F | CH₂ | CH | N | Cl | H | CF₃ | H |
| 15 | 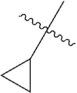 [SMILES: *C1CC1] | 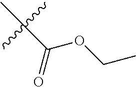 [SMILES: *C(=O)OCC] | Cl | F | CH₂ | CH | N | Cl | H | CF₃ | H |
| 16 | F | 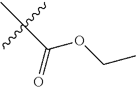 [SMILES: *C(=O)OCC] | Cl | F | CH₂ | CH | N | Cl | H | CF₃ | H |
| 17 | Cl | 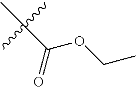 [SMILES: *C(=O)OCC] | Cl | F | CH₂ | CH | N | Cl | H | CF₃ | H |
| 18 | 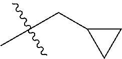 [SMILES: *CC1CC1] | 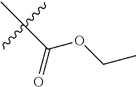 [SMILES: *C(=O)OCC] | Cl | F | CH₂ | CH | N | Cl | H | CF₃ | H |
| 19 | CF₃ | 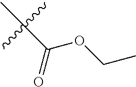 [SMILES: *C(=O)OCC] | Cl | F | CH₂ | CH | N | Cl | H | CF₃ | H |
| 20 | CH₂Br | 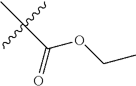 [SMILES: *C(=O)OCC] | Cl | F | CH₂ | CH | N | Cl | H | CF₃ | H |
| 21 | OH | 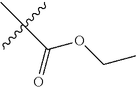 [SMILES: *C(=O)OCC] | Cl | F | CH₂ | CH | N | Cl | H | CF₃ | H |
| 22 | OCH₃ | 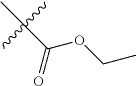 [SMILES: *C(=O)OCC] | Cl | F | CH₂ | CH | N | Cl | H | CF₃ | H |
| 23 | OCH₂CH₃ | 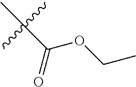 [SMILES: *C(=O)OCC] | Cl | F | CH₂ | CH | N | Cl | H | CF₃ | H |
| 24 | CH₂OH | 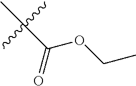 [SMILES: *C(=O)OCC] | Cl | F | CH₂ | CH | N | Cl | H | CF₃ | H |
| 25 | CH₂OCH₃ | 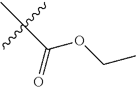 [SMILES: *C(=O)OCC] | Cl | F | CH₂ | CH | N | Cl | H | CF₃ | H |
| 26 | SCH₃ | 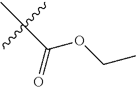 [SMILES: *C(=O)OCC] | Cl | F | CH₂ | CH | N | Cl | H | CF₃ | H |
| 27 | 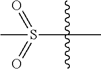 [SMILES: *S(C)(=O)=O] | 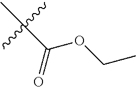 [SMILES: *C(=O)OCC] | Cl | F | CH₂ | CH | N | Cl | H | CF₃ | H |
| 28 | NH₂ | 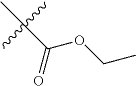 [SMILES: *C(=O)OCC] | Cl | F | CH₂ | CH | N | Cl | H | CF₃ | H |
| 29 | NHCH₃ | 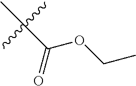 [SMILES: *C(=O)OCC] | Cl | F | CH₂ | CH | N | Cl | H | CF₃ | H |
| 30 | N(CH₃)₂ | 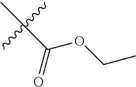 [SMILES: *C(=O)OCC] | Cl | F | CH₂ | CH | N | Cl | H | CF₃ | H |
| 31 | CN | 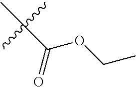 [SMILES: *C(=O)OCC] | Cl | F | CH₂ | CH | N | Cl | H | CF₃ | H |
| 32 | CH₂CN | 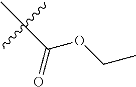 [SMILES: *C(=O)OCC] | Cl | F | CH₂ | CH | N | Cl | H | CF₃ | H |
| 33 | 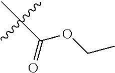 [SMILES: *C(=O)OCC] | 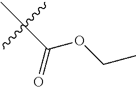 [SMILES: *C(=O)OCC] | Cl | F | CH₂ | CH | N | Cl | H | CF₃ | H |
| 34 | 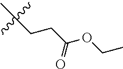 [SMILES: *CCC(=O)OCC] | 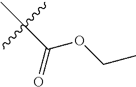 [SMILES: *C(=O)OCC] | Cl | F | CH₂ | CH | N | Cl | H | CF₃ | H |
| 35 | 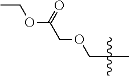 [SMILES: *COCC(=O)OCC] | 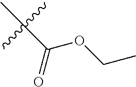 [SMILES: *C(=O)OCC] | Cl | F | CH₂ | CH | N | Cl | H | CF₃ | H |
| 36 | 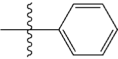 [SMILES: *c1ccccc1] | 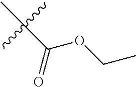 [SMILES: *C(=O)OCC] | Cl | F | CH₂ | CH | N | Cl | H | CF₃ | H |
| 37 | 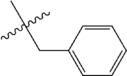 [SMILES: *Cc1ccccc1] | 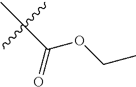 [SMILES: *C(=O)OCC] | Cl | F | CH₂ | CH | N | Cl | H | CF₃ | H |
| 38 | CH₃ | 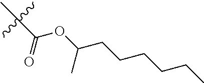 [SMILES: *C(=O)OC(C)CCCCCC] | Cl | F | CH₂ | CH | N | Cl | H | CF₃ | H |
| 39 | CH₃ | 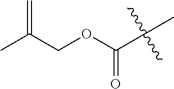 [SMILES: *C(=O)OCC(=C)C] | Cl | F | CH₂ | CH | N | Cl | H | CF₃ | H |
| 40 | CH₃ | 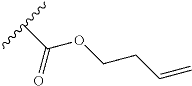 [SMILES: *C(=O)OCCC=C] | Cl | F | CH₂ | CH | N | Cl | H | CF₃ | H |
| 41 | CH₃ | 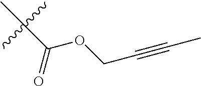 [SMILES: *C(=O)OCC#CC] | Cl | F | CH₂ | CH | N | Cl | H | CF₃ | H |
| 42 | CH₃ | 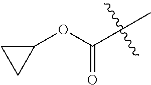 [SMILES: *C(=O)OC1CC1] | Cl | F | CH₂ | CH | N | Cl | H | CF₃ | H |
| 43 | CH₃ | 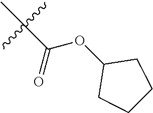 [SMILES: *C(=O)OC1CCCC1] | Cl | F | CH₂ | CH | N | Cl | H | CF₃ | H |
| 44 | CH₃ | 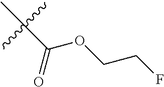 [SMILES: *C(=O)OCCF] | Cl | F | CH₂ | CH | N | Cl | H | CF₃ | H |
| 45 | CH₃ | 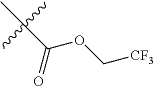 [SMILES: *C(=O)OCC(F)(F)F] | Cl | F | CH₂ | CH | N | Cl | H | CF₃ | H |
| 46 | CH₃ | 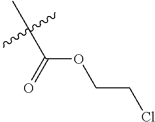 [SMILES: *C(=O)OCCCl] | Cl | F | CH₂ | CH | N | Cl | H | CF₃ | H |
| 47 | CH₃ | 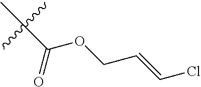 [SMILES: *C(=O)OC/C=C/Cl] | Cl | F | CH₂ | CH | N | Cl | H | CF₃ | H |
| 48 | CH₃ | 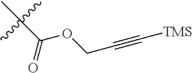 [SMILES: *C(=O)OCC#C[Si](C)(C)C] | Cl | F | CH₂ | CH | N | Cl | H | CF₃ | H |
| 49 | CH₃ | 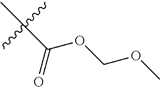 [SMILES: *C(=O)OCOC] | Cl | F | CH₂ | CH | N | Cl | H | CF₃ | H |
| 50 | CH₃ | 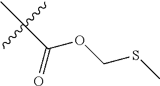 [SMILES: *C(=O)OCSC] | Cl | F | CH₂ | CH | N | Cl | H | CF₃ | H |
| 51 | CH₃ | 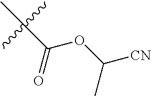 [SMILES: *C(=O)OC(C)C#N] | Cl | F | CH₂ | CH | N | Cl | H | CF₃ | H |
| 52 | CH₃ | 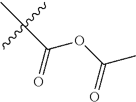 [SMILES: *C(=O)OC(C)=O] | Cl | F | CH₂ | CH | N | Cl | H | CF₃ | H |
| 53 | CH₃ | 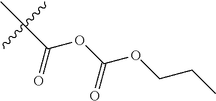 [SMILES: *C(=O)OC(=O)OCCC] | Cl | F | CH₂ | CH | N | Cl | H | CF₃ | H |
| 54 | CH₃ | 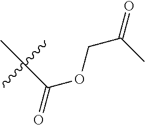 [SMILES: *C(=O)OCC(C)=O] | Cl | F | CH₂ | CH | N | Cl | H | CF₃ | H |
| 55 | CH₃ | 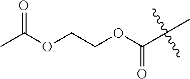 [SMILES: *C(=O)OCCOC(C)=O] | Cl | F | CH₂ | CH | N | Cl | H | CF₃ | H |
| 56 | CH₃ | 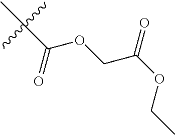 [SMILES: *C(=O)OCC(=O)OCC] | Cl | F | CH₂ | CH | N | Cl | H | CF₃ | H |
| 57 | CH₃ | 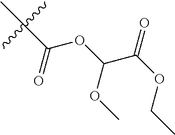 [SMILES: *C(=O)OC(OC)C(=O)OCC] | Cl | F | CH₂ | CH | N | Cl | H | CF₃ | H |
| 58 | CH₃ | 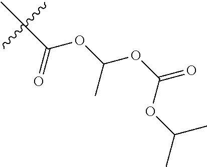 [SMILES: *C(=O)OC(C)OC(=O)OC(C)C] | Cl | F | CH₂ | CH | N | Cl | H | CF₃ | H |
| 59 | CH₃ | 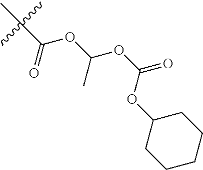 [SMILES: *C(=O)OC(C)OC(=O)OC1CCCCC1] | Cl | F | CH₂ | CH | N | Cl | H | CF₃ | H |
| 60 | CH₃ | 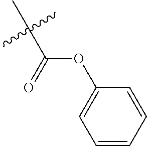 [SMILES: *C(=O)Oc1ccccc1] | Cl | F | CH₂ | CH | N | Cl | H | CF₃ | H |
| 61 | CH₃ | 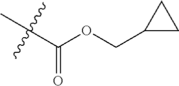 [SMILES: *C(=O)OCC1CC1] | Cl | F | CH₂ | CH | N | Cl | H | CF₃ | H |
| 62 | CH₃ | 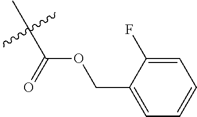 [SMILES: *C(=O)OCc1ccccc1F] | Cl | F | CH₂ | CH | N | Cl | H | CF₃ | H |
| 63 | CH₃ | 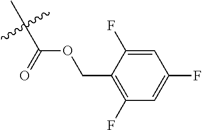 [SMILES: *C(=O)OCc1c(F)cc(F)cc1F] | Cl | F | CH₂ | CH | N | Cl | H | CF₃ | H |
| 64 | CH₃ | 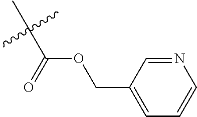 [SMILES: *C(=O)OCc1cccnc1] | Cl | F | CH₂ | CH | N | Cl | H | CF₃ | H |
| 65 | CH₃ | 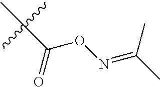 [SMILES: *C(=O)ON=C(C)C] | Cl | F | CH₂ | CH | N | Cl | H | CF₃ | H |
| 66 | CH₃ | 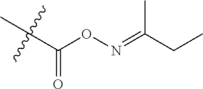 [SMILES: *C(=O)O/N=C(\C)CC] | Cl | F | CH₂ | CH | N | Cl | H | CF₃ | H |
| 67 | CH₃ | 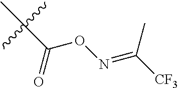 [SMILES: *C(=O)O/N=C(\C)C(F)(F)F] | Cl | F | CH₂ | CH | N | Cl | H | CF₃ | H |
| 68 | CH₃ | 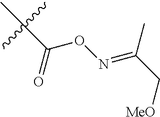 [SMILES: *C(=O)O/N=C(\C)COC] | Cl | F | CH₂ | CH | N | Cl | H | CF₃ | H |
| 69 | CH₃ | 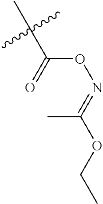 [SMILES: *C(=O)O/N=C(\C)OCC] | Cl | F | CH₂ | CH | N | Cl | H | CF₃ | H |
| 70 | CH₃ | 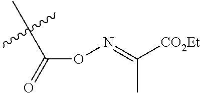 [SMILES: *C(=O)O/N=C(\C)C(=O)OCC] | Cl | F | CH₂ | CH | N | Cl | H | CF₃ | H |
| 71 | CH₃ | 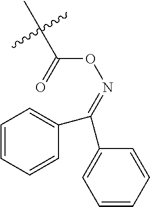 [SMILES: *C(=O)ON=C(c1ccccc1)c1ccccc1] | Cl | F | CH₂ | CH | N | Cl | H | CF₃ | H |
| 72 | CH₃ | 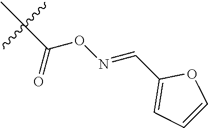 [SMILES: *C(=O)O/N=C/c1ccco1] | Cl | F | CH₂ | CH | N | Cl | H | CF₃ | H |
| 73 | CH₃ | 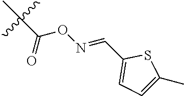 [SMILES: *C(=O)O/N=C/c1ccc(C)s1] | Cl | F | CH₂ | CH | N | Cl | H | CF₃ | H |
| 74 | CH₃ | 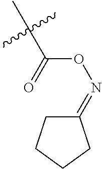 [SMILES: *C(=O)ON=C1CCCC1] | Cl | F | CH₂ | CH | N | Cl | H | CF₃ | H |
| 75 | CH₃ | 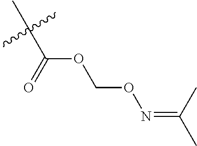 [SMILES: *C(=O)OCON=C(C)C] | Cl | F | CH₂ | CH | N | Cl | H | CF₃ | H |
| 76 | CH₃ | 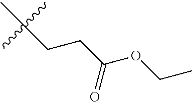 [SMILES: *CCC(=O)OCC] | Cl | F | CH₂ | CH | N | Cl | H | CF₃ | H |
| 77 | CH₃ | 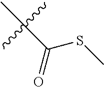 [SMILES: *C(=O)SC] | Cl | F | CH₂ | CH | N | Cl | H | CF₃ | H |
| 78 | CH₃ | 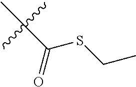 [SMILES: *C(=O)SCC] | Cl | F | CH₂ | CH | N | Cl | H | CF₃ | H |
| 79 | CH₃ | 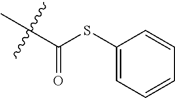 [SMILES: *C(=O)Sc1ccccc1] | Cl | F | CH₂ | CH | N | Cl | H | CF₃ | H |
| 80 | CH₃ | 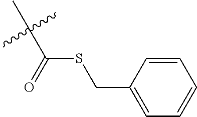 [SMILES: *C(=O)SCc1ccccc1] | Cl | F | CH₂ | CH | N | Cl | H | CF₃ | H |
| 81 | CH₃ | 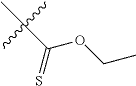 [SMILES: *C(=S)OCC] | Cl | F | CH₂ | CH | N | Cl | H | CF₃ | H |
| 82 | CH₃ | 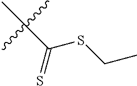 [SMILES: *C(=S)SCC] | Cl | F | CH₂ | CH | N | Cl | H | CF₃ | H |
| 83 | CH₃ | 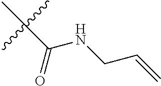 [SMILES: *C(=O)NCC=C] | Cl | F | CH₂ | CH | N | Cl | H | CF₃ | H |
| 84 | CH₃ | 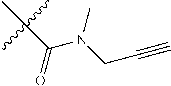 [SMILES: *C(=O)N(C)CC#C] | Cl | F | CH₂ | CH | N | Cl | H | CF₃ | H |
| 85 | CH₃ | 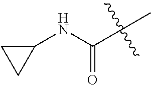 [SMILES: *C(=O)NC1CC1] | Cl | F | CH₂ | CH | N | Cl | H | CF₃ | H |
| 86 | CH₃ | 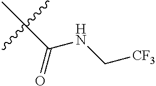 [SMILES: *C(=O)NCC(F)(F)F] | Cl | F | CH₂ | CH | N | Cl | H | CF₃ | H |
| 87 | CH₃ | 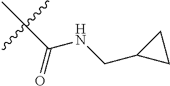 [SMILES: *C(=O)NCC1CC1] | Cl | F | CH₂ | CH | N | Cl | H | CF₃ | H |
| 88 | CH₃ | 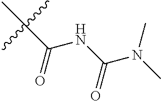 [SMILES: *C(=O)NC(=O)N(C)C] | Cl | F | CH₂ | CH | N | Cl | H | CF₃ | H |
| 89 | CH₃ | 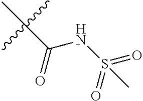 [SMILES: *C(=O)NS(C)(=O)=O] | Cl | F | CH₂ | CH | N | Cl | H | CF₃ | H |
| 90 | CH₃ | 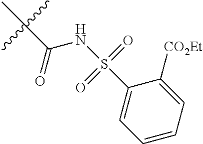 [SMILES: *C(=O)NS(=O)(=O)c1ccccc1C(=O)OCC] | Cl | F | CH₂ | CH | N | Cl | H | CF₃ | H |
| 91 | CH₃ | 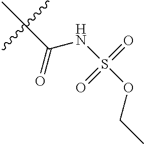 [SMILES: *C(=O)NS(=O)(=O)OCC] | Cl | F | CH₂ | CH | N | Cl | H | CF₃ | H |
| 92 | CH₃ | 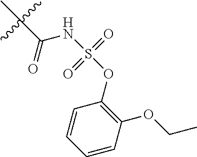 [SMILES: *C(=O)NS(=O)(=O)Oc1ccccc1OCC] | Cl | F | CH₂ | CH | N | Cl | H | CF₃ | H |
| 93 | CH₃ | 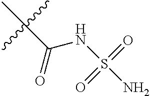 [SMILES: *C(=O)NS(N)(=O)=O] | Cl | F | CH₂ | CH | N | Cl | H | CF₃ | H |
| 94 | CH₃ | 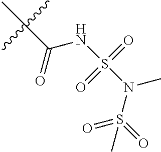 [SMILES: *C(=O)NS(=O)(=O)N(C)S(C)(=O)=O] | Cl | F | CH₂ | CH | N | Cl | H | CF₃ | H |
| 95 | CH₃ | 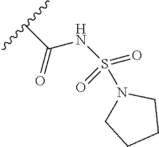 [SMILES: *C(=O)NS(=O)(=O)N1CCCC1] | Cl | F | CH₂ | CH | N | Cl | H | CF₃ | H |
| 96 | CH₃ | 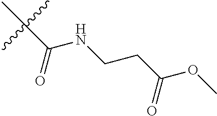 [SMILES: *C(=O)NCCC(=O)OC] | Cl | F | CH₂ | CH | N | Cl | H | CF₃ | H |
| 97 | CH₃ | 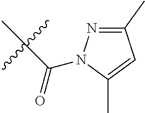 [SMILES: *C(=O)n1nc(C)cc1C] | Cl | F | CH₂ | CH | N | Cl | H | CF₃ | H |
| 98 | CH₃ | 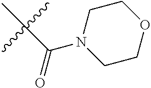 [SMILES: *C(=O)N1CCOCC1] | Cl | F | CH₂ | CH | N | Cl | H | CF₃ | H |
| 99 | CH₃ | 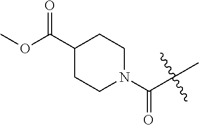 [SMILES: *C(=O)N1CCC(C(=O)OC)CC1] | Cl | F | CH₂ | CH | N | Cl | H | CF₃ | H |
| 100 | CH₃ | 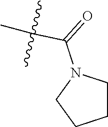 [SMILES: *C(=O)N1CCCC1] | Cl | F | CH₂ | CH | N | Cl | H | CF₃ | H |
| 101 | CH₃ | 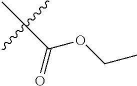 [SMILES: *C(=O)OCC] | Cl | F | CH₂ | CH | N | Cl | H | CF₃ | CH₃ |
| 102 | CH₃ | 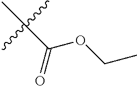 [SMILES: *C(=O)OCC] | Cl | F | CH₂ | CH | N | Cl | H | OCH₃ | H |
| 103 | CH₃ | 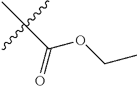 [SMILES: *C(=O)OCC] | Cl | F | CH₂ | CH | N | Cl | H | SCH₃ | H |
| 104 | CH₃ | 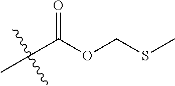 [SMILES: *C(=O)OCSC] | Cl | F | CH₂ | CH | N | Cl | H | 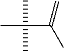 | H |
| 105 | H | 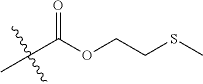 [SMILES: *C(=O)OCCSC] | Cl | F | CH₂ | CH | N | Cl | H | CF₃ | H |
| 106 | CH₃ | 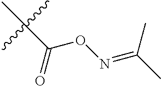 [SMILES: *C(=O)ON=C(C)C] | Cl | F | CH₂ | CH | N | Cl | H | CF₃ | H |
| 107 | CH₃ | 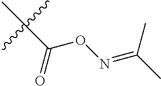 [SMILES: *C(=O)ON=C(C)C] | Cl | F | CH₂ | CH | CH | Cl | H | CF₃ | H |
| 108 | CH₃ | 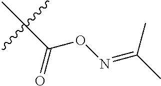 [SMILES: *C(=O)ON=C(C)C] | Cl | F | CH₂ | N | CH | Cl | H | CF₃ | H |
| 109 | CH₃ | 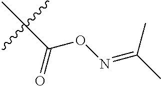 [SMILES: *C(=O)ON=C(C)C] | Cl | F | CH₂ | N | N | Cl | H | CF₃ | H |
| 110 | CH₃ | 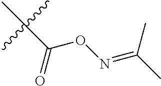 [SMILES: *C(=O)ON=C(C)C] | Cl | F | CH₂ | CH | N⁺—O⁻ | Cl | H | CF₃ | H |
| 111 | CH₃ | 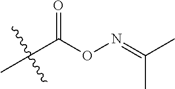 [SMILES: *C(=O)ON=C(C)C] | Cl | F | CH(CH₃) | CH | N | Cl | H | CF₃ | H |
| 112 | CH₃ | 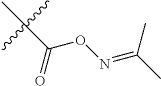 [SMILES: *C(=O)ON=C(C)C] | Cl | F | CH₂ | CH | N | Cl | H | H | H |
| 113 | CH₃ | 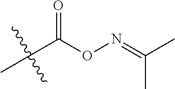 [SMILES: *C(=O)ON=C(C)C] | Cl | F | CH₂ | CH | N | Cl | H | 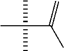 | H |
| 114 | CH₃ | 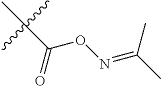 [SMILES: *C(=O)ON=C(C)C] | Cl | F | CH₂ | CH | N | Cl | H | 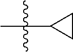 [SMILES: *C1CC1] | H |
| 115 | CH₃ | 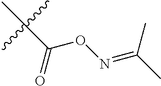 [SMILES: *C(=O)ON=C(C)C] | Cl | F | CH₂ | CH | N | Cl | H | F | H |
| 116 | CH₃ | 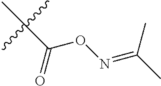 [SMILES: *C(=O)ON=C(C)C] | Cl | F | CH₂ | CH | N | Cl | H | Cl | H |
| 117 | CH₃ | 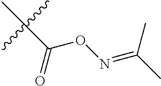 [SMILES: *C(=O)ON=C(C)C] | Cl | F | CH₂ | CH | N | Cl | H | Br | H |
| 118 | CH₃ | 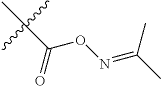 [SMILES: *C(=O)ON=C(C)C] | Cl | F | CH₂ | CH | N | Cl | H | 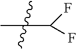 [SMILES: *C(F)F] | H |
| 119 | CH₃ | 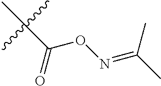 [SMILES: *C(=O)ON=C(C)C] | Cl | F | CH₂ | CH | N | Cl | H | 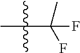 [SMILES: *C(C)(F)F] | H |
| 120 | CH₃ | 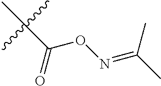 [SMILES: *C(=O)ON=C(C)C] | Cl | F | CH₂ | CH | N | Cl | H | 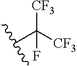 [SMILES: *C(F)(C(F)(F)F)C(F)(F)F] | H |
| 121 | CH₃ | 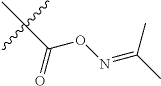 [SMILES: *C(=O)ON=C(C)C] | Cl | F | CH₂ | CH | N | Cl | H | OCH₃ | H |
| 122 | CH₃ | 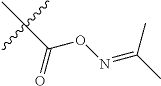 [SMILES: *C(=O)ON=C(C)C] | Cl | F | CH₂ | CH | N | Cl | H | 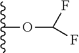 [SMILES: *OC(F)F] | H |
| 123 | CH₃ | 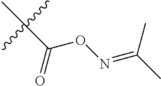 [SMILES: *C(=O)ON=C(C)C] | Cl | F | CH₂ | CH | N | Cl | H | 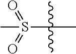 [SMILES: *S(C)(=O)=O] | H |
| 124 | CH₃ | 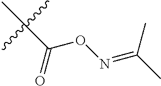 [SMILES: *C(=O)ON=C(C)C] | Cl | F | CH₂ | CH | N | Cl | H | CN | H |
| 125 | CH₃ | 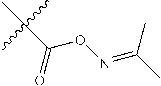 [SMILES: *C(=O)ON=C(C)C] | Cl | F | CH₂ | CH | N | Cl | H | NO₂ | H |
| 126 | H | 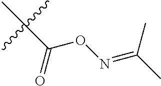 [SMILES: *C(=O)ON=C(C)C] | Cl | F | CH₂ | CH | N | Cl | H | CF₃ | H |
| 127 | CF₃ | 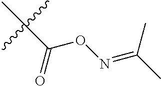 [SMILES: *C(=O)ON=C(C)C] | Cl | F | CH₂ | CH | N | Cl | H | CF₃ | H |
| 128 | 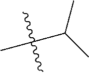 [SMILES: *C(C)C] | 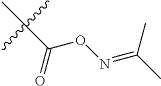 [SMILES: *C(=O)ON=C(C)C] | Cl | F | CH₂ | CH | N | Cl | H | CF₃ | H |
| 129 | 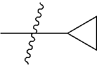 [SMILES: *C1CC1] | 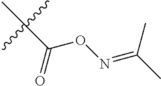 [SMILES: *C(=O)ON=C(C)C] | Cl | F | CH₂ | CH | N | Cl | H | CF₃ | H |
| 130 | CH₃ | 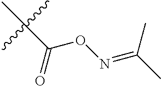 [SMILES: *C(=O)ON=C(C)C] | Cl | F | CH₂ | CH | N | F | H | CF₃ | H |
| 131 | CH₃ | 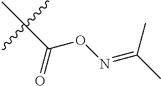 [SMILES: *C(=O)ON=C(C)C] | Cl | F | CH₂ | CH | N | F | F | 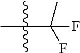 [SMILES: *C(C)(F)F] | H |
| 132 | CH₃ | 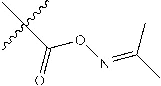 [SMILES: *C(=O)ON=C(C)C] | Cl | F | CH₂ | CH | N | F | H | F | H |
| 133 | CH₃ | 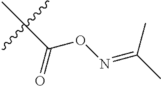 [SMILES: *C(=O)ON=C(C)C] | Cl | F | CH₂ | CH | N | F | H | Cl | H |
| 134 | CH₃ | 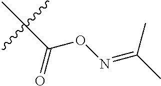 [SMILES: *C(=O)ON=C(C)C] | Cl | F | CH₂ | CH | N | F | H | CH₃ | H |
| 135 | CH₃ | 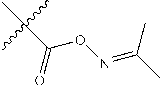 [SMILES: *C(=O)ON=C(C)C] | Cl | F | CH₂ | CH | N | H | H | CF₃ | H |
| 136 | CH₃ | 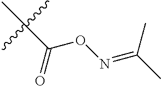 [SMILES: *C(=O)ON=C(C)C] | Cl | F | CH₂ | CH | N | CH₃ | H | CF₃ | H |
| 137 | CH₃ | 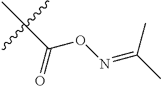 [SMILES: *C(=O)ON=C(C)C] | Cl | F | CH₂ | CH | N | 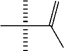 | H | CF₃ | H |
| 138 | CH₃ | 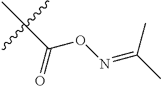 [SMILES: *C(=O)ON=C(C)C] | Cl | F | CH₂ | CH | N | OCH₃ | H | CF₃ | H |
| 139 | CH₃ | 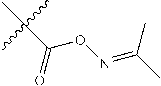 [SMILES: *C(=O)ON=C(C)C] | CF₃ | F | CH₂ | CH | N | Cl | H | CF₃ | H |
| 140 | CH₃ | 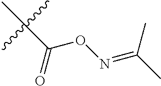 [SMILES: *C(=O)ON=C(C)C] | CN | F | CH₂ | CH | N | Cl | H | CF₃ | H |
| 141 | CH₃ | 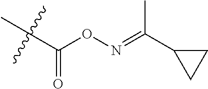 [SMILES: *C(=O)O/N=C(\C)C1CC1] | Cl | F | CH₂ | CH | N | Cl | H | CF₃ | H |

TABLE 2
¹H NMR of Compounds

| No. | ¹H NMR |
|---|---|
| 1 | ¹H NMR (300 MHz, Chloroform-d) δ 8.78 (s, 1H), 7.79 (d, J = 7.5 Hz, 1H), 7.31 (d, J = 9.0 Hz, 1H), 4.27 (q, J = 7.0 Hz, |
| | 2H), 4.00 (d, J = 17.5 Hz, 1H), 3.40 (d, J = 17.5 Hz, 1H), 2.62 (s, 3H), 1.72 (s, 3H), 1.32 (t, J = 7.0 Hz, 3H). |
| 10 | ¹H NMR (300 MHz, Chloroform-d) δ 8.85 (s, 1H), 8.07 (s, 1H), 7.72 (d, J = 7.2 Hz, 1H), 7.52 (d, J = 8.7 Hz, 1H), 4.01 (d, J = |
| | 17.4 Hz, 1H), 3.83 (q, J = 7.2 Hz, 2H), 3.43 (d, J = 17.4 Hz, 1H), 1.74 (s, 3H), 1.26 (t, J = 7.2 Hz, 3H). |
| 12 | ¹H NMR (300 MHz, DMSO-d₆) δ 9.09 (s, 1H), 8.68 (s, 1H), 7.88-7.81 (m, 2H), 4.18 (q, J = 6.9 Hz, 2H), 3.79 (d, J = 18.0 |
| | Hz, 1H), 3.58 (d, J = 18.0 Hz, 1H), 2.33-2.23 (m, 1H), 1.21 (t, J = 6.9 Hz, 3H), 0.91-0.85 (m, 6H). |
| 15 | ¹H NMR (300 MHz, Chloroform-d) δ 8.85 (s, 1H), 8.07 (s, 1H), 7.56 (d, J = 7.0 Hz, 1H), 7.36 (d, J = 9.0 Hz, 1H), 5.14- |
| | 5.12 (m, 1H), 4.18 (q, J = 7.0 Hz, 2H), 3.60-3.58 (m, 1H), 1.39-1.30 (m, 4H), 0.88-0.56 (m, 4H). |
| 43 | ¹H NMR (300 MHz, DMSO-d₆) δ 9.10 (m, 1H), 8.70 (s, 1H), 7.85 (d, J = 9.0 Hz, 1H), 7.82 (d, J = 7.0 Hz, 1H), 5.19-5.08 |
| | (m, 1H), 3.92-3.82 (m, 1H), 3.52-3.45 (m, 1H), 1.85-1.59 (m, 8H), 1.56 (s, 3H). |
| 44 | ¹H NMR (300 MHz, Chloroform-d) δ 8.86 (s, 1H), 8.07 (s, 1H), 7.83 (d, J = 7.5 Hz, 1H), 7.32 (d, J = 9.0 Hz, 1H), 4.80- |
| | 4.70 (m, 1H), 4.60-4.52 (m, 1H), 4.53-4.47 (m, 1H), 4.45-4.36 (m, 1H), 4.02 (d, J = 17.5 Hz, 1H), 3.44 (d, J = 17.5 Hz, |
| | 1H), 1.75 (s, 3H). |
| 45 | ¹H NMR (300 MHz, Chloroform-d) δ 8.85 (s, 1H), 8.07 (s, 1H), 7.83 (d, J = 7.5 Hz, 1H), 7.33 (d, J = 9.0 Hz, 1H), 4.62- |
| | 4.50 (m, 2H), 4.02 (d, J = 17.5 Hz, 1H), 3.50 (d, J = 17.5 Hz, 1H), 1.78 (s, 3H). |
| 49 | ¹H NMR (300 MHz, CDCl₃) δ 8.87 (s, 1H), 8.06 (s, 1H), 7.82 (d, J = 7.0 Hz, 1H), 7.31 (d, J = 9.0 Hz, 1H), 5.36-5.31 (m, |
| | 2H), 4.04 (d, J = 17.5 Hz, 1H), 3.49-3.46 (m, 3H), 3.42 (d, J = 17.5 Hz, 1H), 1.74 (s, 3H). |
| 50 | ¹H NMR (300 MHz, CDCl₃) δ 8.86 (d, J = 0.7 Hz, 1H), 8.07 (d, J = 1.9 Hz, 1H), 7.84 (d, J = 7.4 Hz, 1H), 7.33 (d, J = 9.1 |
| | Hz, 1H), 5.27 (d, J = 1.8 Hz, 2H), 4.04 (d, J = 17.4 Hz, 1H), 3.43 (d, J = 17.4 Hz, 1H), 2.26 (s, 3H), 1.75 (s, 4H). |
| 51 | ¹H NMR (300 MHz, DMSO-d₆) δ 9.10 (s, 1H), 8.70 (s, 1H), 7.87 (d, J = 7.5 Hz, 1H), 7.32 (d, J = 9.0 Hz, 1H), 5.58-5.52 |
| | (m, 1H), 3.99-3.90 (m, 1H), 3.60-3.55 (m, 1H), 1.63 (s, 3H), 1.58 (d, J = 7.0 Hz, 3H). |
| 65 | ¹H NMR (300 MHz, Chloroform-d) δ 8.85 (s, 1H), 8.06 (s, 1H), 7.81 (d, J = 7.5 Hz, 1H), 7.32 (d, J = 9.0 Hz, 1H), 4.11 (d, J = |
| | 17.5 Hz, 1H), 3.45 (d, J = 17.5 Hz, 1H), 2.06 (d, J = 5.5 Hz, 6H), 1.80 (s, 3H). |
| 67 | ¹H NMR (300 MHz, Chloroform-d) δ 8.85 (s, 1H), 8.07 (s, 1H), 7.82 (d, J = 7.2 Hz, 1H), 7.34 (d, J = 9.0 Hz, 1H), 4.14 (d, J = |
| | 17.4 Hz, 1H), 3.50 (d, J = 17.4 Hz, 1H), 2.23 (s, 3H), 1.84 (s, 3H). |
| 68 | ¹H NMR (300 MHz, Chloroform-d) δ 8.85 (s, 1H), 8.07 (s, 1H), 7.82 (d, J = 7.5 Hz, 1H), 7.33 (d, J = 9.0 Hz, 1H), 4.17- |
| | 4.05 (m, 3H), 3.47 (d, J = 17.4 Hz, 1H), 3.35 (s, 3H), 2.09 (s, 3H), 1.81 (s, 3H). |
| 69 | ¹H NMR (300 MHz, Chloroform-d) δ 8.85 (s, 1H), 8.06 (s, 1H), 7.81 (d, J = 7.2 Hz, 1H), 7.32 (d, J = 9.0 Hz, 1H), 4.20 (q, J = |
| | 7.2 Hz, 2H), 4.10 (d, J = 17.4 Hz, 1H), 3.44 (d, J = 17.4 Hz, 1H), 2.07 (s, 3H), 1.79 (s, 3H), 1.32 (t, J = 7.2 Hz, 3H). |
| 70 | ¹H NMR (300 MHz, Chloroform-d) δ 8.79 (s, 1H), 8.01 (s, 1H), 7.83-7.69 (m, 1H), 7.36-7.22 (m, 1H), 4.36-4.20 (m, 2H), |
| | 4.41-3.90 (m, 1H), 3.53-3.33 (m, 1H), 2.25-2.01 (m, 3H), 1.82-1.64 (m, 3H), 1.34-1.25 (m, 3H). |
| 71 | ¹H NMR (300 MHz, Chloroform-d) δ 8.87 (s, 1H), 8.08 (s, 1H), 7.71 (d, J = 7.2 Hz, 1H), 7.59 (d, J = 9.0 Hz, 1H), 7.46- |
| | 7.30 (m, 10H), 3.86 (d, J = 17.4 Hz, 1H), 3.34 (d, J = 17.4 Hz, 1H), 1.62 (s, 3H). |
| 72 | ¹H NMR (300 MHz, Chloroform-d) δ 8.85 (s, 1H), 8.34 (s, 1H), 8.06 (s, 1H), 7.86-7.74 (m, 1H), 7.58 (d, J = 2.4 Hz, 1H), |
| | 7.37-7.24 (m, 1H), 6.95-6.93 (m, 1H), 6.53-6.50 (m, 1H), 4.14 (d, J = 17.4 Hz, 1H), 3.51 (d, J = 17.4 Hz, 1H), 1.81 (s, |
| | 3H). |
| 73 | ¹H NMR (300 MHz, Chloroform-d) δ 8.86 (s, 1H), 8.34 (s, 1H), 8.06 (s, 1H), 7.82 (d, J = 7.2 Hz, 1H), 7.31 (d, J = 9.0, 1H), |
| | 6.95-6.83 (m, 2H), 4.12 (d, J = 17.4 Hz, 1H), 3.49 (d, J = 17.4 Hz, 1H), 2.35 (s, 3H), 1.81 (s, 3H). |
| 74 | ¹H NMR (300 MHz, Chloroform-d) δ 8.85 (s, 1H), 8.06 (s, 1H), 7.82 (d, J = 7.2 Hz, 1H), 7.32 (d, J = 9.0 Hz, 1H), 4.11 (d, J = |
| | 17.4 Hz, 1H), 3.45 (d, J = 17.4 Hz, 1H), 2.64-2.55 (m, 4H), 1.84-1.80 (m, 4H), 1.78 (s, 3H). |
| 76 | ¹H NMR (300 MHz, DMSO-d₆) δ 9.10 (s, 1H), 8.70 (s, 1H), 7.83 (s, 1H), 7.80 (d, J = 3.0 Hz, 1H), 4.06-3.99 (m, 2H), 3.37 |
| | (d, J = 17.7 Hz, 1H), 3.21 (d, J = 17.7 Hz, 1H), 2.41-2.31 (m, 2H), 1.95-1.90 (m, 2H), 1.36 (s, 3H), 1.14 (t, J = 9.0 Hz, |
| | 3H). |
| 78 | ¹H NMR (300 MHz, CDCl₃) δ 8.85 (s, 1H), 8.06 (s, 1H), 7.83 (d, J = 7.5 Hz, 1H), 7.31 (d, J = 9.0 Hz, 1H), 3.86 (d, J = 17.5 |
| | Hz, 1H), 3.44 (d, J = 17.5 Hz, 1H), 2.85 (dd, J = 7.5 Hz, 2H), 1.71 (s, 3H), 1.23 (d, J = 7.5 Hz, 3H). |
| 97 | ¹H NMR (300 MHz, CDCl₃) δ 8.85 (s, 1H), 8.06 (s, 1H), 7.83 (d, J = 7.5 Hz, 1H), 7.31 (d, J = 9.0 Hz, 1H), 5.97 (s, 1H), |
| | 4.16 (d, J = 18.0 Hz, 1H), 3.71 (d, J = 18.0 Hz, 1H), 2.55 (s, 3H), 2.22 (s, 3H), 1.96 (s, 3H). |
| 102 | ¹H NMR (300 MHz, Chloroform-d) δ 8.30 (d, J = 2.7 Hz, 1H), 7.77 (d, J = 7.5 Hz, 1H), 7.33 (d, J = 2.7 Hz, 1H), 7.27 (d, J = |
| | 8.7 Hz, 1H), 4.26 (q, J = 6.9 Hz, 2H), 3.99 (d, J = 17.7 Hz, 1H), 3.91 (s, 3H), 3.39 (d, J = 17.7 Hz, 1H), 1.71 (s, 3H), 1.32 |
| | (t, J = 6.9 Hz, 3H). |
| 104 | ¹H NMR (300 MHz, Chloroform-d) δ 8.68 (d, J = 2.1 Hz, 1H), 7.84 (d, J = 2.1 Hz, 1H), 7.81 (d, J = 7.5, Hz, 1H), 7.29 (d, J = |
| | 9.3 Hz, 1H), 5.53 (d, J = 1.8 Hz, 1H), 5.29 (d, J = 1.8 Hz, 1H), 5.26 (s, 2H), 4.02 (d, J = 17.4 Hz, 1H), 3.42 (d, J = 17.4 |
| | Hz, 1H), 2.26 (s, 3H), 2.19 (s, 3H), 1.74 (s, 3H). |
| 105 | ¹H NMR (300 MHz, Chloroform-d) δ 8.86 (s, 1H), 8.07 (s, 1H), 7.85 (d, J = 7.2 Hz, 1H), 7.34 (d, J = 9.0 Hz, 1H), 5.29- |
| | 5.17 (m, 1H), 4.39 (t, J = 6.9 Hz, 2H), 3.89-3.76 (m, 2H), 2.78 (t, J = 6.9 Hz, 2H), 2.16 (s, 3H). |
| 111 | ¹H NMR (300 MHz, DMSO-d₆) δ 9.09 (s, 1H), 8.70 (s, 1H), 7.89-7.77 (m, 2H), 4.20-4.13 (m, 1H), 2.07-2.06 (m, 3H), |
| | 2.01-2.00 (m, 3H), 1.47 (s, 3H), 1.01 (d, J = 7.5 Hz, 3H). |
| 113 | ¹H NMR (300 MHz, Chloroform-d) δ 8.67 (d, J = 2.1 Hz, 1H), 7.84 (d, J = 2.1 Hz, 1H), 7.79 (d, J = 7.5 Hz, 1H), 7.29 (d, J = |
| | 9.3 Hz, 1H), 5.52 (d, J = 1.8 Hz, 1H), 5.29 (d, J = 1.8 Hz, 1H), 4.10 (d, J = 17.7 Hz, 1H), 3.44 (d, J = 17.7 Hz, 1H), 2.19- |
| | 2.17 (t, J = 2.4 Hz, 3H), 2.07 (s, 3H), 2.05 (s, 3H), 1.79 (s, 3H). |
| 127 | ¹H NMR (300 MHz, Chloroform-d) δ 8.87 (s, 1H), 8.08 (s, 1H), 7.84 (d, J = 7.2 Hz, 1H), 7.37 (d, J = 9.0 Hz, 1H), 4.34 (d, J = |
| | 18.3 Hz, 1H), 4.01 (d, J = 18.3 Hz, 1H), 2.10 (d, J = 3.0 Hz, 6H). |
| 129 | ¹H NMR (300 MHz, Chloroform-d) δ 8.79 (s, 1H), 8.00 (s, 1H), 7.70 (d, J = 7.5 Hz, 1H), 7.26 (d, J = 9.0 Hz, 1H), 4.06 (d, J = |
| | 17.4 Hz, 1H), 3.48 (d, J = 17.4 Hz, 1H), 2.01 (d, J = 3.0 Hz, 6H), 1.31-1.29 (m, 1H), 0.77-0.69 (m, 2H), 0.59-0.48 (m, |
| | 2H). |
| 139 | ¹H NMR (300 MHz, Chloroform-d) δ 8.88 (s, 1H), 8.10 (s, 1H), 7.72 (d, J = 7.2 Hz, 1H), 7.61 (d, J = 9.5 Hz, 1H), 3.95 (d, J = |
| | 17.4 Hz, 1H), 3.29 (d, J = 17.4 Hz, 1H), 2.12-2.03 (m, 6H), 1.80 (s, 3H). |
| 140 | ¹H NMR (300 MHz, Chloroform-d) δ 8.88 (s, 1H), 8.11 (s, 1H), 7.95 (d, J = 6.6 Hz, 1H), 7.60 (d, J = 8.7 Hz, 1H), 4.15 (d, J = |
| | 17.4 Hz, 1H), 3.51 (d, J = 17.4 Hz, 1H), 2.07 (d, J = 3.6 Hz, 6H), 1.83 (s, 3H). |
| 141 | ¹H NMR (300 MHz, Chloroform-d) δ 8.84 (s, 1H), 8.06 (s, 1H), 7.80 (d, J = 7.2 Hz, 1H), 7.32 (d, J = 9.0 Hz, 1H), 4.12 (d, J = |
| | 17.4 Hz, 1H), 3.46 (d, J = 17.4 Hz, 1H), 2.44-2.38 (m, 1H), 1.82 (s, 3H), 1.70 (s, 3H), 0.99-0.94 (m, 2H), 0.90-0.84 (m, |
| | 2H). |

Several methods for preparing the compounds of the present invention are illustrated in detail in the following solutions and examples. Raw materials can be purchased on the market or can be prepared by methods known in literature or as shown in the detailed description. A person skilled in the art should understand that other synthetic routes may also be used to synthesize the compounds of the present invention. Although the specific raw materials and conditions in the synthetic routes have been illustrated below, they may be easily replaced with other similar raw materials and conditions. Various isomers and the like of the compounds resulted from these modifications or variants of the preparation methods of the present invention are all included in the scope of the present invention. In addition, the preparation methods as described below can be further modified according to the disclosure of the present invention using conventional chemical methods well known to a person skilled in the art. For example, the protection for an appropriate group during the reaction, etc.

The examples of the methods provided below are used to enhance further understanding of the preparation method of the present invention. The specific substances, types and conditions used are determined to further illustrate the present invention and are not intended to limit its reasonable scope. Reagents used in the synthesis of the compounds shown in the following tables are either commercially available or can be easily prepared by a person skilled in the art.

Examples of representative compounds are as follows. The synthesis methods of other compounds are similar and will not be described in detail here.

### 1. Synthesis of Compound 10

(1) Compound 10-1 (52.3 g, 0.21 mmol), ethylene glycol (25.6 g, 0.42 mol), and p-toluenesulfonic acid (3.4 g, 0.02 mol) were dissolved in toluene (500 mL) and reacted at 120° C. for 8 h. When liquid chromatography detected that the raw materials had been completely consumed, the reaction solution was cooled down followed by concentration, added with water, extracted three times with EA, and washed three times with saturated saline solution. The organic phase was dried and concentrated to obtain the crude product which was then stirred and purified through a silica gel column to obtain Compound 10-2 (55.2 g, 0.18 mol) with a yield of 85%.

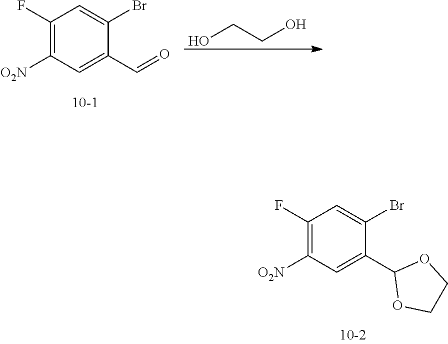

(2) Compound 10-2 (55.2 g, 0.18 mol) was dissolved in 500 mL of ethanol and 100 mL of water, added with iron powder (50 g, 0.9 mol), NH₄Cl (19 g, 0.36 mmol), heated to 60° C. and reacted for 2 h. The liquid chromatography detected the completion of the reaction. The reaction solution was cooled and suction-filtered with a layer of diatomaceous earth on the bottom of the filter flask. The mother liquor was concentrated to obtain the crude product which was then added with ethyl acetate and washed with water to separate liquid phases. The organic phase was concentrated to obtain the crude product which was then stirred and purified through a silica gel column to result in Compound 10-3 (30 g, 0.11 mol), with a yield of 61%.

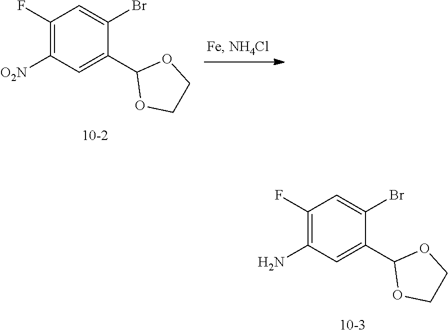 [SMILES: O=[N+]([O-])c1cc(C2OCCO2)c(Br)cc1F ; Nc1cc(C2OCCO2)c(Br)cc1F]

(3) Compound 10-3 (10 g, 38 mmol) was dissolved in 200 mL of acetonitrile, added with bis(pinacolato)diborane 10-4 (48 g, 1.90 mol), and then slowly added dropwise with tert-butyl nitrite (7.8 g, 76 mmol). The system was heated to 60° C. and reacted for 12 h. The LCMS detected disappearance of the raw materials. The reaction solution was concentrated to obtain the crude product which was then extracted with water and ethyl acetate. The organic phase was dried, concentrated, stirred, and purified through a silica gel column to obtain Compound 10-5 (5.5 g, 14.7 mmol) with a yield of 39%.

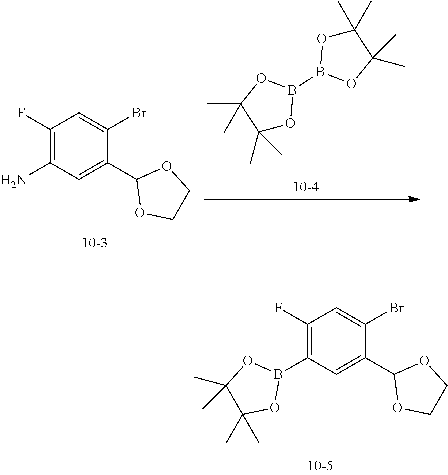

(4) Compound 10-6 (0.4 g, 1.86 mmol), borate 10-5 (1.04 g, 2.80 mmol), and potassium carbonate (0.78 g, 5.6 mmol) were added into a clean flask, added with 20 mL of 1,4-dioxane and 2 mL of water, and then protected by nitrogen and evacuated for 2-3 times. After evacuation, the system was added with 1,1′-bis(diphenylphosphino)ferrocene-palladium(II)dichloride dichloromethane complex (44.74 mg, 54.79 μmol), evacuated again, heated to 100° C. and stayed overnight. Upon the completion of the reaction detected by In-Process Control, the solution was treated by decompressing distillation to dry the solvent, washed with water, and extracted for 2-3 times with EA. The EA phase was then washed for 2-3 times with saturated saline solution and added with anhydrous sodium sulfate for drying. The sample was added with silica gel, stirred, and purified by normal phase chromatography to obtain Compound 10-7 (600 mg, 1.42 mmol).

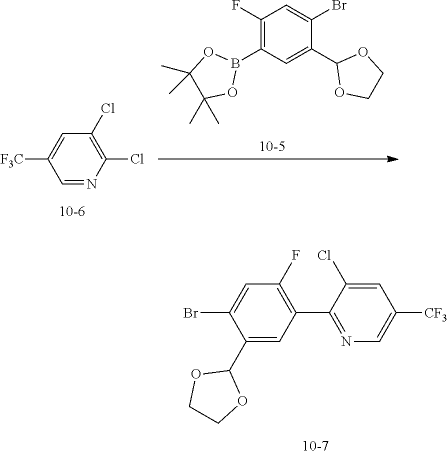 [SMILES: FC(F)(F)c1cnc(Cl)c(Cl)c1 ; CC1(C)OB(c2cc(C3OCCO3)c(Br)cc2F)OC1(C)C ; Fc1cc(Br)c(C2OCCO2)cc1-c1ncc(C(F)(F)F)cc1Cl]

(5) Compound 10-7 (0.6 g, 1.42 mmol) and hydroxylamine hydrochloride (0.48 g, 7.1 mmol) were added in a clean flask, added with a mixed solvent of ethanol and water (10 mL:2 mL), and stirred for 3 h at 80° C. Upon the completion of the reaction detected by In-Process Control, the sample was directly stirred and purified by normal phase chromatography to obtain Compound 10-8 (540 mg, 1.36 mmol).

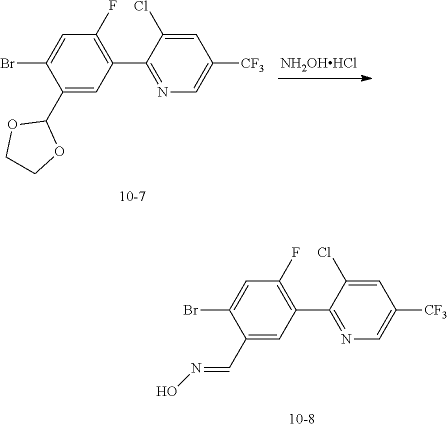

(6) Compound 10-8 (0.54 g, 1.36 mmol) and NCS (0.36 g, 2.72 mmol) were added in a clean flask, added with 10 mL of DMF for dissolution, and stirred at 40° C. for 3 h. Upon the completion of the reaction detected by In-Process Control, the sample was washed with water, and then added with EA for extraction (extracted 3 times with a suitable amount of EA). Next, the EA phase was washed twice with saturated saline solution and spin-dried to obtain the white solid 10-9 (580 mg, 1.35 mmol).

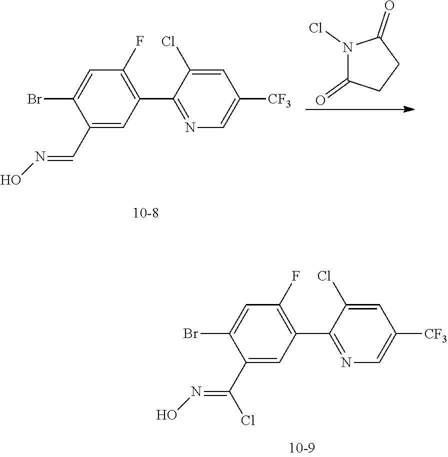 [SMILES: O/N=C/c1cc(-c2ncc(C(F)(F)F)cc2Cl)c(F)cc1Br ; O=C1CCC(=O)N1Cl ; O/N=C(\Cl)c1cc(-c2ncc(C(F)(F)F)cc2Cl)c(F)cc1Br]

(7) Compound 10-9 (0.8 g, 0.69 mmol) and triethylamine (0.14 g, 1.38 mmol) were dissolved in DCM, and in ice batch, added dropwise with the dichloromethane solution of Compound 10-10 (0.2 g, 0.46 mmol). After the addition, the ice-water bath was removed and the system stayed at room temperature overnight. Upon the completion of the reaction detected by in-process control, the sample was directly stirred and purified by normal phase chromatography to obtain Compound 10 (70 mg, 0.14 mmol).

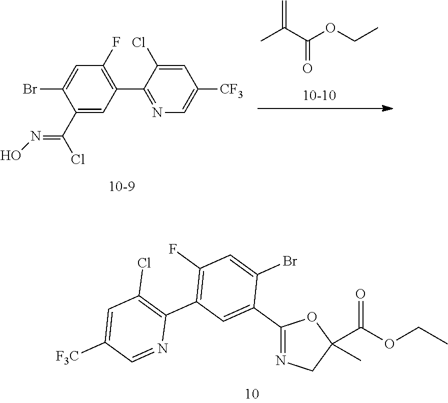

### 2. Synthesis of Compound 104

(1) A 100-mL single-necked flask was added in with Compound 104-1 (2 g, 10.62 mmol, 1.0 eq), 16 mL of 1,4-dioxane and 4 mL of water, cesium fluoride (3.23 g, 21.23 mmol, 2.0 eq), Compound 104-2 (2.99 g, 15.92 mmol, 1.5 eq), and a catalytic amount of Pd(dppf)Cl₂ (0.087 g, 0.096 mmol, 0.01 eq). After the addition, the air in the system was replaced with argon gas for 3 times. The reaction solution was stirred overnight at 100° C. The LCMS detected that the reaction of the raw materials was essentially complete with the formation of a major new peak. The reaction solution was rotary-evaporated to remove most of the solvent and 100 mL of water and ethyl acetate (100 mL×3) was added for extraction. The organic phase was washed with saturated saline solution (100 mL×3), concentrated, then stirred and purified through a silica gel column to obtain Compound 104-3 (2.5 g, 80% yield, pale yellow solid).

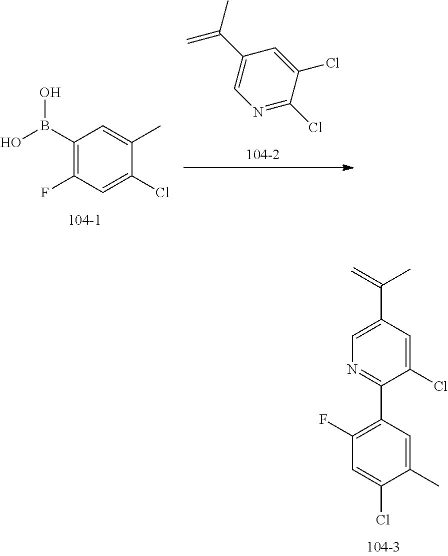 [SMILES: Cc1cc(B(O)O)c(F)cc1Cl ; C=C(C)c1cnc(Cl)c(Cl)c1 ; C=C(C)c1cnc(-c2cc(C)c(Cl)cc2F)c(Cl)c1]

(2) A 100-mL single-necked flask was sequentially added in with Compound 104-3 (2.5 g, 8.44 mmol, 1.0 eq), AIBN (0.45 g, 2.72 mmol, 0.2 eq), NBS (2.25 g, 12.66 mmol, 1.5 eq), and 30 mL of acetonitrile. Next, the air in the system was replaced with argon gas for 3 times. The reaction solution was stirred overnight at 80° C. The LCMS detected that the reaction of the raw materials was essentially complete with the formation of a major new peak. The reaction solution was rotary-evaporated to remove most of the solvent and 20 mL of water and ethyl acetate (50 mL×3) was added for extraction. The organic phase was washed with saturated saline solution (20 mL×3) and concentrated to obtain the crude product 104-4 (2.3 g, crude product, yellow solid) which was directly used in the next step.

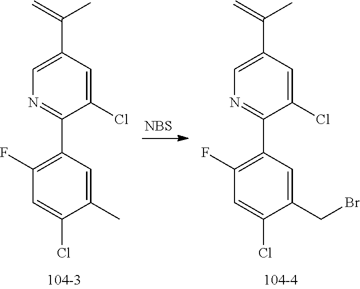

(3) A 100-mL single-necked flask was sequentially added in with Compound 104-4 (2.3 g, 6.13 mmol, 1.0 eq), N-methylmorpholine oxide (1.44 g, 12.23 mmol, 2 eq), and 30 mL of acetonitrile. The reaction solution was stirred overnight at room temperature. The LCMS detected that the reaction of the raw materials was essentially complete with the formation of a major new peak. The reaction solution was rotary-evaporated to remove most of the solvent and 20 mL of water and ethyl acetate (50 mL×3) was added for extraction. The organic phase was washed with saturated saline solution (20 mL×3) and concentrated to obtain the crude product 104-5 (1.5 g, crude product, yellow solid) which was directly used in the next step.

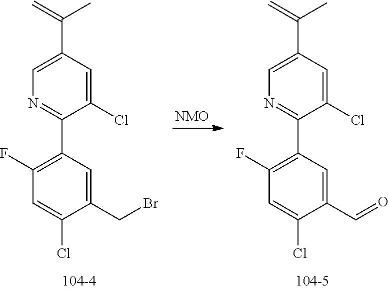

(4) A 100-mL single-necked flask was sequentially added in with Compound 104-5 (1.5 g, 4.84 mmol, 1.0 eq), hydroxylamine hydrochloride (0.34 g, 4.84 mmol, 1 eq), and 25 mL of ethanol. The reaction solution was stirred for three hours at room temperature. The LCMS detected that the reaction of the raw materials was essentially complete with the formation of a major new peak. The reaction solution was rotary-evaporated to remove the solvent and added with 20 mL of water and ethyl acetate (50 mL×3) for extraction. The organic phase was washed with saturated saline solution (20 mL×3) and concentrated to obtain the crude product 104-6 (1.2 g, crude product, pale yellow solid) which was directly used in the next step.

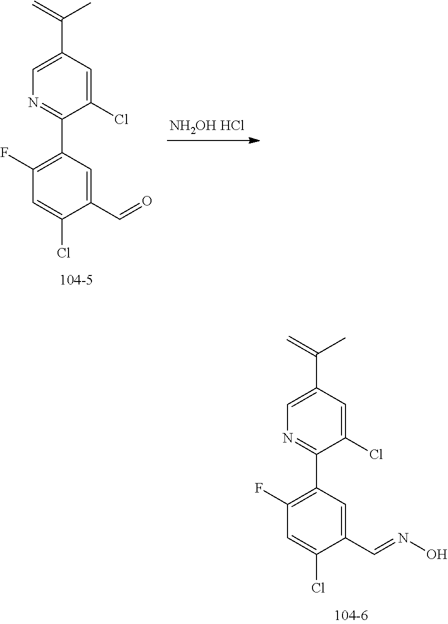

(5) A 100-mL single-necked flask was sequentially added in with Compound 104-6 (1.2 g, 3.69 mmol, 1.0 eq), NCS (0.52 g, 3.87 mmol, 1.05 eq), and 20 mL of DMF. The reaction solution was stirred at room temperature for three hours. The LCMS detected that the reaction of the raw materials was essentially complete with the formation of a major new peak. The system was added with 100 mL of water and ethyl acetate (100 mL×3) for extraction. The organic phase was washed with saturated saline solution (20 mL×3) and concentrated to obtain the crude product 104-7 (0.9 g, crude product, yellow solid) which was directly used in the next step.

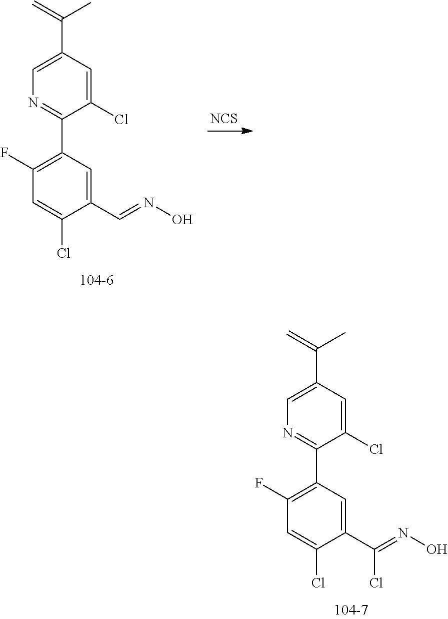 [SMILES: C=C(C)c1cnc(-c2cc(/C=N/O)c(Cl)cc2F)c(Cl)c1 ; "NCS" ; C=C(C)c1cnc(-c2cc(/C(Cl)=N/O)c(Cl)cc2F)c(Cl)c1]

(6) At 25° C., a 100-mL single-necked flask was sequentially added in with ethyl methacrylate (0.75 g, 7.51 mmol, 3 eq), triethylamine (0.25 g, 2.5 mmol, 1 eq), and 15 mL of dichloromethane, added dropwise with the dichloromethane solution of Compound 104-7 (0.9 g, 2.5 mmol, 1 eq), and stirred at room temperature for 5 h. The LCMS detected that the reaction of the raw materials was essentially complete with the formation of a major new peak. The system was added with water (100 mL×3) for extraction. The organic phase was washed with saturated saline solution (20 mL×3) and concentrated to obtain the crude product 104-8 (0.7 g, crude product, yellow oily matter) which was directly used in the next step.

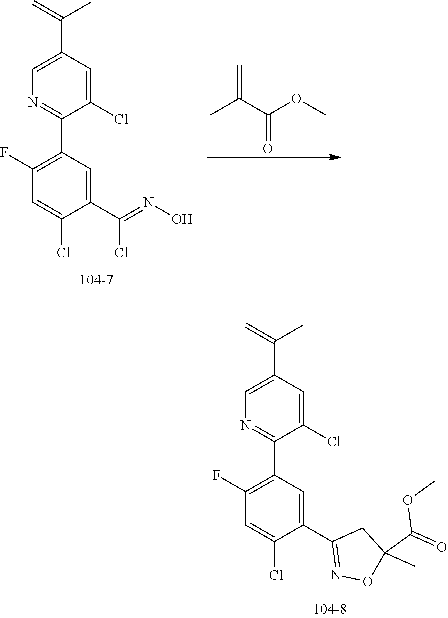

(7) At 25° C., a 100-mL single-necked flask was sequentially added in with Compound 104-8 (0.8 g, 1.89 mmol, 1 eq), lithium hydroxide (0.14 g, 5.7 mmol, 3 eq), 15 mL of THF and 5 mL of water and stirred at room temperature for 5 h. The LCMS detected that the reaction of the raw materials was essentially complete with the formation of a major new peak. The pH was adjusted to weak acidity. The system was added with 100 mL of water and ethyl acetate (100 mL×3) for extraction. The organic phase was washed with saturated saline solution (20 mL×3) and concentrated to obtain the crude product 104-9 (0.7 g, crude product, yellow oily matter) which was directly used in the next step.

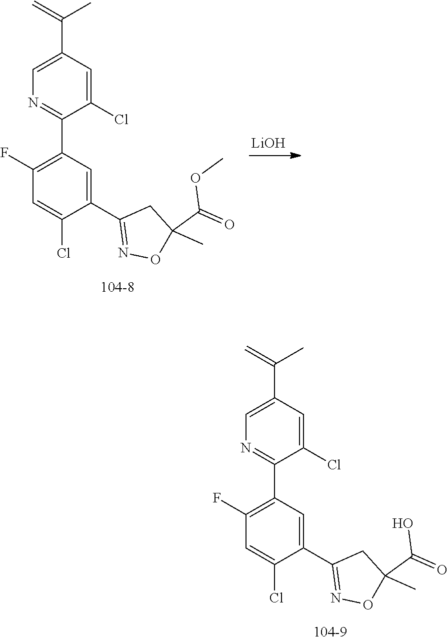

(8) A 100-mL single-necked flask was added in with Compound 104-9 (0.1 g, 0.24 mmol, 1.0 eq), chloromethyl methyl sulfide (0.047 g, 0.49 mmol, 2 eq), anhydrous potassium carbonate (0.1 g, 0.74 mmol, 3.0 eq), and 10 mL of DMF and reacted at 45° C. for 3 h. The LCMS detected that the reaction of the raw materials was essentially complete with the formation of a major new peak. The system was added with 20 mL of water and ethyl acetate (20 mL×3) for extraction. The organic phase was washed with saturated saline solution (20 mL×3), concentrated, then stirred and purified through a silica gel column to obtain Compound 104 (0.05 g, 44% yield, colorless oily matter).

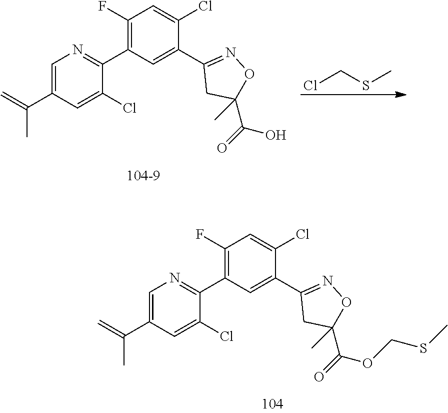

### Evaluation of Biological Activity:

The activity level criteria for plant damage (i.e., growth control rate) are as follows:
Level 10: completely dead;
Level 9: growth control rate greater than or equal to 90% and less than 100%;
Level 8: growth control rate greater than or equal to 80% and less than 90%;
Level 7: growth control rate greater than or equal to 70% and less than 80%;
Level 6: growth control rate greater than or equal to 60% and less than 70%;
Level 5: growth control rate greater than or equal to 50% and less than 60%;
Level 4: growth control rate greater than or equal to 40% and less than 50%;
Level 3: growth control rate greater than or equal to 30% and less than 40%;
Level 2: growth control rate greater than or equal to 20% and less than 30%;
Level 1: growth control rate less than 20%;
Level 0: no effect.

The above growth control rates are fresh weight control rates.

### Post-Emergence Test Experiment:

Monocotyledonous and dicotyledonous weed seeds (Descurainia sophia, Capsella bursa-pastoris, Abutilon theophrasti, Galium spurium, Stellaria media, Lithospermum arvense, Rorippa indica, Alopecurus aequalis, Alopecurus japonicus, Eleusine indica, Beckmannia syzigachne, Sclerochloa dura, Erigeron canadensis, Phleum paniculatum, Veronica polita, Bromus japonicus, Aegilops tauschii, Phalaris arundinacea, Amaranthus retroflexus, Chenopodium, Commelina communis, Cichorium endivia, Convolvulus arvensis, Cirsium arvense, Solanum nigrum, Acalypha australis, Digitaria sanguinalis, Echinochloa crus-galli, Setaria viridis, Setaria pumila, Leptochloa chinensis, Monochoria vaginalis, Sagittaria trifolia, Schoenoplectiella juncoides, Cyperus rotundus, Cyperus iria, Cyperus difformis, Fimbristylis, Portulaca oleracea, Xanthium sibiricum, Ipomoea nil, Eschenbachia japonica, etc.) and major crop seeds (wheat, maize, paddy rice, soybean, cotton, oilseed rape, millet, sorghum, potato, sesame, castor plant, etc.) were placed in plastic pots filled with soil and then covered with 0.5-2 cm of soil to allow them to grow in a favorable greenhouse environment. Two weeks after sowing, the test plants were treated at the stage of 2-3 leaves. The test compounds of the present invention were individually dissolved with acetone, then added with Tween® 80, with 1.5 L/ha of methyl oleate emulsifiable concentrate as a synergist, diluted into solutions of certain concentrations using certain amount of water, and sprayed to the plants using a spray tower. After drug application, the experimental results on weeds were collected after 3 weeks of cultivation in a greenhouse. The compounds were applied at 500, 250, 125, 60, 15, and 7.5 g a.i./ha in triplicate, and the results were averaged. Representative data are listed in Table 3.

TABLE 3
Results of post-emergence test on weeds

| | Echinochloa | Amaranthus | Veronica | Abutilon | Digitaria | Setaria | Dosage (g |
|---|---|---|---|---|---|---|---|
| No. | crus-galli | retroflexus | polita | theophrasti | sanguinalis | viridis | a.i./ha) |
| 1 | 10 | 10 | 10 | 10 | 10 | 10 | 60 |
| 10 | 10 | 10 | 10 | 10 | 10 | 10 | 60 |
| 12 | 10 | 10 | 10 | 10 | 10 | 10 | 60 |
| 15 | 10 | 10 | 10 | 10 | 10 | 10 | 15 |
| 43 | 10 | 10 | 10 | 10 | 10 | 10 | 15 |
| 44 | 10 | 10 | 10 | 10 | 10 | 10 | 15 |
| 45 | 10 | 10 | 10 | 10 | 10 | 10 | 60 |
| 49 | 10 | 10 | 10 | 10 | 10 | 10 | 15 |
| 50 | 10 | 10 | 10 | 10 | 10 | 10 | 15 |
| 51 | 10 | 10 | 10 | 10 | 10 | 10 | 15 |
| 65 | 10 | 10 | 10 | 10 | 10 | 10 | 15 |
| 67 | 10 | 10 | 10 | 10 | 10 | 10 | 60 |
| 68 | 10 | 10 | 10 | 10 | 10 | 10 | 60 |
| 69 | 10 | 10 | 10 | 10 | 10 | 10 | 60 |
| 70 | 10 | 10 | 10 | 10 | 10 | 10 | 60 |
| 71 | 10 | 10 | 10 | 10 | 10 | 10 | 60 |
| 72 | 10 | 10 | 10 | 10 | 10 | 10 | 60 |
| 73 | 10 | 10 | 10 | 10 | 10 | 10 | 60 |
| 74 | 10 | 10 | 10 | 10 | 10 | 10 | 60 |
| 76 | 10 | 10 | 10 | 10 | 10 | 10 | 15 |
| 78 | 10 | 10 | 10 | 10 | 10 | 10 | 15 |
| 97 | 10 | 10 | 10 | 10 | 10 | 10 | 15 |
| 102 | 10 | 10 | 10 | 10 | 10 | 10 | 60 |
| 104 | 10 | 10 | 10 | 10 | 10 | 10 | 60 |
| 105 | 10 | 10 | 10 | 10 | 10 | 10 | 60 |
| 111 | 10 | 10 | 10 | 10 | 10 | 10 | 60 |
| 113 | 10 | 10 | 10 | 10 | 10 | 10 | 60 |
| 127 | 10 | 10 | 10 | 10 | 10 | 10 | 60 |
| 129 | 10 | 10 | 10 | 10 | 10 | 10 | 60 |
| 139 | 10 | 10 | 10 | 10 | 10 | 10 | 60 |
| 140 | 10 | 10 | 10 | 10 | 10 | 10 | 60 |
| 141 | 10 | 10 | 10 | 10 | 10 | 10 | 60 |

### Pre-Emergence Test Experiment

Monocotyledonous and dicotyledonous weed seeds as well as major crop seeds (wheat, maize, paddy rice, soybean, cotton, oilseed rape, millet, sorghum) were placed in plastic pots filled with soil and then covered with 0.5-2 cm of soil. The test compounds of the present invention were individually dissolved with acetone, then added with Tween® 80, diluted with certain amount of water to obtain solutions of certain concentrations, and sprayed immediately after sowing. After drug application, the experimental results were observed after 4 weeks of cultivation in a greenhouse, showing that most of the drugs of the present invention exhibited outstanding effects at the dose of 250 g a.i./ha., especially on weeds such as Echinochloa crus-galli, Digitaria sanguinalis, Abutilon theophrasti, etc., and many of the compounds had great selectivity to maize, wheat, paddy rice, and soybean.

Meanwhile, it has been found by experimentations on major weeds in wheat fields and paddy rice fields that the compounds of the present invention generally have good weed control effects; in particular, it has been noticed that the compounds have extremely high activity on broad-leaf weeds and Cyperaceae that are resistant to ALS inhibitors, for example, Sagittaria trifolia, Schoenoplectiella juncoides, Cyperus difformis, Descurainia sophia, Capsella bursa-pastoris, Lithospermum arvense, Galium spurium, Cyperus rotundus, etc., which have excellent commercial values.

Meanwhile, it has been found by extensive experimentations that many of the compounds of the present invention and compositions thereof have good selectivity to gramineae grasses such as zoysia grass, bermuda grass, tall fescue, bluegrass, ryegrass, seashore paspalum, etc., and can prevent or eliminate many key gramineous weeds and broad-leaf weeds. The experiments on sugarcane, soybean, cotton, oil sunflower, potato, fruit trees, vegetables and the like using different drug application methods also demonstrated excellent selectivity and commercial values.

1. A heterocycle-substituted aromatic compound, as shown in the general formula I: 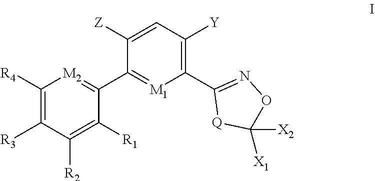
wherein,
Y represents halogen, haloalkyl, cyano, nitro, or amino;
Z represents hydrogen, halogen, or hydroxyl;
M₁ and M₂ each independently represent CR₅ or N(O)ₘ;
Q represents CX₃X₄;
R₁, R₂, R₃, R₄, and R₅ each independently represent hydrogen, halogen, hydroxyl, mercapto, formyl, hydroxylalkyl, nitro, cyano, alkyl, alkenyl, alkynyl, cycloalkyl, cycloalkylalkyl, cycloalkenyl, cycloalkenylalkyl, —OR₁₁, —SR₁₁, —(SO)R₁₁, —(SO₂)R₁₁, —(SO₂)OR₁₁, —O(SO₂)R₁₁, —N(R₁₂)₂, phenyl, or benzyl, wherein,
the “alkyl”, “alkenyl”, “alkynyl”, “cycloalkyl”, “cycloalkylalkyl”, “cycloalkenyl”, or “cycloalkenylalkyl” is each independently unsubstituted or substituted by halogen; the “phenyl” or “benzyl” is each independently unsubstituted or substituted by at least one group selected from halogen, cyano, nitro, alkyl, haloalkyl, alkoxycarbonyl, alkylsulfanyl, alkylsulfonyl, alkoxy, and haloalkoxy;
X₁, X₂, X₃, and X₄ each independently represent hydrogen, halogen, nitro, cyano, thiocyanato, hydroxyl, mercapto, sulfo, formyl, haloformyl, azido, alkyl, alkenyl, alkynyl, cycloalkyl, cycloalkylalkyl, cycloalkenyl, cycloalkenylalkyl, —PO(OR′)₂, —OR″, —(CO)R″, —SR″, —(SO)R″, —(SO₂)R″, —Si(R″)₃, —O(CO)R″, —O—(SO₂)R″, —S(CO)R″, —(SO₂)OR″, —O(CO)OR″, —(CO)(CO)OR″, 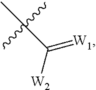
—CR′═N—OH, —CR′═N—O—R″, heterocyclyl, heterocyclylalkyl, aryl, arylalkyl, amino, aminoalkyl, aminocarbonylalkyl, aminocarbonyloxyalkyl, aminothiocarbonyloxyalkyl, aminosulfonyl, or aminosulfonyloxyalkyl, wherein,
the “alkyl”, “alkenyl”, or “alkynyl” is each independently unsubstituted or substituted by at least one group selected from halogen, cyano, hydroxyl, mercapto, carboxyl, —OR″, —(CO)R″, —SR″, —(SO₂)R″, —O(CO)H, —O(CO)R″, —O—(SO₂)R″, —(CO)OR″, —O(CO)OR″, —O(CO)(CO)OH, —O(CO)(CO)OR″, —O-alkyl-(CO)OH, and —O-alkyl-(CO)OR″;
the “cycloalkyl”, “cycloalkylalkyl”, “cycloalkenyl”, “cycloalkenylalkyl”, “heterocyclyl”, “heterocyclylalkyl”, “aryl”, or “arylalkyl” is each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₄, —SR₁₄, —(CO)OR₁₄, —(SO₂)R₁₄, —N(R₁₄)₂, and —O-alkyl-(CO)OR₁₄, or two adjacent carbon atoms on the ring form a fused ring with —OCH₂CH₂— or —OCH₂O— that is unsubstituted or substituted by halogen;
the “amino”, “aminoalkyl”, “aminocarbonylalkyl”, “aminocarbonyloxyalkyl”, “aminothiocarbonyloxyalkyl”, “aminosulfonyl”, or “aminosulfonyloxyalkyl” is each independently unsubstituted or substituted by one or two groups selected from —R₁₁, —OR₁₁, —(CO)R₁₁, —(CO)OR₁₁, -alkyl-(CO)OR₁₁, —(SO₂)R₁₁, —(SO₂)OR₁₁, -alkyl-(SO₂)R₁₁, —(CO)N(R₁₂)₂, and —(SO₂)N(R₁₂)₂;
R′ each independently represents hydrogen, halogen, alkoxy, alkoxyalkyl, alkyl, alkenyl, alkynyl, cycloalkyl, cycloalkylalkyl, cycloalkenyl, cycloalkenylalkyl, aryl, arylalkyl, heterocyclyl, or heterocyclylalkyl, wherein, the “alkyl”, “alkenyl”, or “alkynyl” is each independently unsubstituted or substituted by halogen; the “cycloalkyl”, “cycloalkylalkyl”, “cycloalkenyl”, “cycloalkenylalkyl”, “aryl”, “arylalkyl”, “heterocyclyl”, or “heterocyclylalkyl” is each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₄, —SR₁₄, —(CO)OR₁₄, —(SO₂)R₁₄, —N(R₁₄)₂, and —O-alkyl-(CO)OR₁₄, or two adjacent carbon atoms on the ring form a fused ring with —OCH₂CH₂— or —OCH₂O— that is unsubstituted or substituted by halogen;
R″ each independently represents alkyl, alkenyl, alkynyl, cycloalkyl, cycloalkylalkyl, cycloalkenyl, cycloalkenylalkyl, aryl, arylalkyl, arylalkenyl, heterocyclyl, heterocyclylalkyl, or heterocyclylalkenyl, wherein, the “alkyl”, “alkenyl”, or “alkynyl” is each independently unsubstituted or substituted by at least one group selected from halogen, cyano, trialkylsilyl, —OR₁₃, —SR₁₃, —O(CO)R₁₃, —(CO)R₁₃, —(CO)OR₁₃, and —O(CO)OR₁₃; the “cycloalkyl”, “cycloalkylalkyl”, “cycloalkenyl”, “cycloalkenylalkyl”, “aryl”, “arylalkyl”, “arylalkenyl”, “heterocyclyl”, “heterocycylalkyl”, or “heterocyclylalkenyl” is each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₄, —SR₁₄, —(CO)OR₁₄, —(SO₂)R₁₄, —N(R₁₄)₂, and —O-alkyl-(CO)OR₁₄, or two adjacent carbon atoms on the ring form a fused ring with —OCH₂CH₂— or —OCH₂O— that is unsubstituted or substituted by halogen;
W₁ represents O, S, or NW₄;
W₂ represents OW₃, SW₃, or NW₄W₅;
W₃ each independently represents hydrogen, alkyl, alkenyl, alkynyl, cycloalkyl, cycloalkenyl, heterocyclyl, aryl, 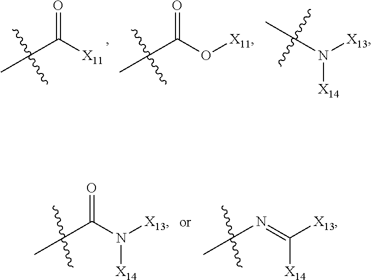
wherein, the “alkyl”, “alkenyl”, or “alkynyl” is each independently unsubstituted or substituted by at least one group selected from halogen, cyano, nitro, cycloalkyl, trialkylsilyl, cycloalkenyl, heterocyclyl, aryl, 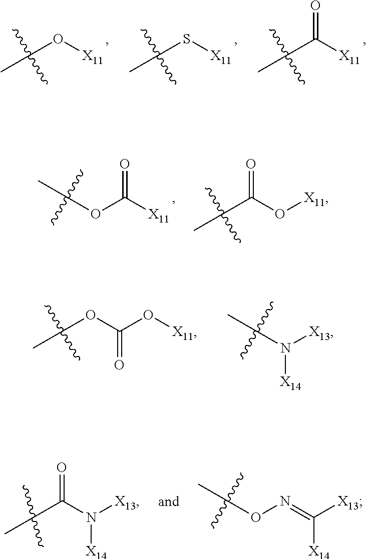
the “cycloalkyl”, “cycloalkenyl”, “heterocyclyl”, or “aryl” is each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₄, —SR₁₄, —(CO)OR₁₄, —(SO₂)R₁₄, —N(R₁₄)₂, and —O-alkyl-(CO)OR₁₄, or two adjacent carbon atoms on the ring form a fused ring with —OCH₂CH₂— or —OCH₂O— that is unsubstituted or substituted by halogen;
W₄ and W₅ each independently represent hydrogen, alkyl, alkenyl, alkynyl, cycloalkyl, cycloalkenyl, heterocyclyl, aryl, 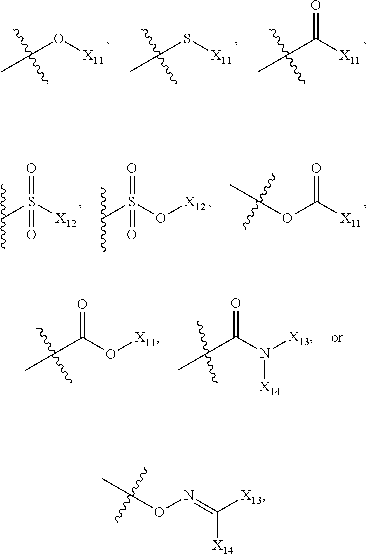
wherein, the “alkyl”, “alkenyl”, or “alkynyl” is each independently unsubstituted or substituted by at least one group selected from halogen, cyano, nitro, cycloalkyl, trialkylsilyl, cycloalkenyl, heterocyclyl, aryl, 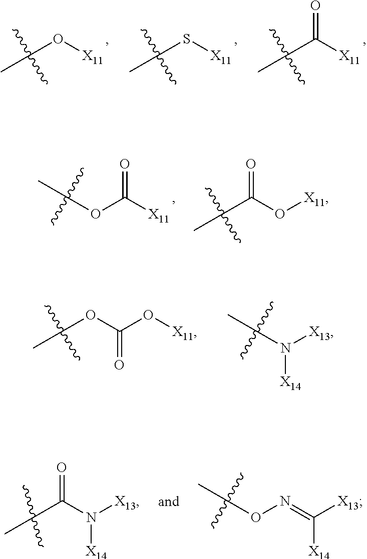
the “cycloalkyl”, “cycloalkenyl”, “heterocyclyl”, or “aryl” is each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₄, —SR₁₄, —(CO)OR₁₄, —(SO₂)R₁₄, —N(R₁₄)₂, and —O-alkyl-(CO)OR₁₄, or two adjacent carbon atoms on the ring form a fused ring with —OCH₂CH₂— or —OCH₂O— that is unsubstituted or substituted by halogen;
or NW₄W₅ represents 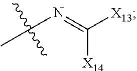
or unsubstituted or substituted heterocyclyl with a nitrogen atom at 1-position;
X₁₁ each independently represents hydrogen, alkyl, alkenyl, alkynyl, cycloalkyl, cycloalkylalkyl, cycloalkenyl, cycloalkenylalkyl, heterocyclyl, heterocyclylalkyl, aryl, or arylalkyl, wherein, the “cycloalkyl”, “cycloalkylalkyl”, “cycloalkenyl”, “cycloalkenylalkyl”, “heterocyclyl”, “heterocyclylalkyl”, “aryl”, or “arylalkyl” is each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₄, —SR₁₄, —(CO)OR₁₄, —(SO₂)R₁₄, —N(R₁₄)₂, and —O-alkyl-(CO)OR₁₄, or two adjacent carbon atoms on the ring form a fused ring with —OCH₂CH₂— or —OCH₂O— that is unsubstituted or substituted by halogen;
X₁₂ each independently represents alkyl, alkenyl, alkynyl, cycloalkyl, cycloalkylalkyl, cycloalkenyl, cycloalkenylalkyl, heterocyclyl, heterocyclylalkyl, aryl, or arylalkyl, wherein, the “cycloalkyl”, “cycloalkylalkyl”, “cycloalkenyl”, “cycloalkenylalkyl”, “heterocyclyl”, “heterocyclylalkyl”, “aryl”, or “arylalkyl” is each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₄, —SR₁₄, —(CO)OR₁₄, —(SO₂)R₁₄, —N(R₁₄)₂, and —O-alkyl-(CO)OR₁₄, or two adjacent carbon atoms on the ring form a fused ring with —OCH₂CH₂— or —OCH₂O— that is unsubstituted or substituted by halogen;
X₁₃ and X₁₄ each independently represent hydrogen, halogen, cyano, alkoxy, alkoxyalkyl, alkylcarbonyl, alkoxycarbonyl, alkylsulfonyl, alkyl, alkenyl, alkynyl, cycloalkyl, cycloalkylalkyl, cycloalkenyl, cycloalkenylalkyl, aryl, arylalkyl, heterocyclyl, or heterocyclylalkyl, or the group CX₁₃X₁₄ together forms an unsubstituted or substituted cyclic structure, or the group NX₁₃X₁₄ together forms an unsubstituted or substituted heterocyclyl with a nitrogen atom at 1-position, wherein, the “alkyl”, “alkenyl”, or “alkynyl” is each independently unsubstituted or substituted by halogen; the “cycloalkyl”, “cycloalkylalkyl”, “cycloalkenyl”, “cycloalkenylalkyl”, “aryl”, “arylalkyl”, “heterocyclyl”, or “heterocyclylalkyl” is each independently unsubstituted or substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₄, —SR₁₄, —(CO)OR₁₄, —(SO₂)R₁₄, —N(R₁₄)₂, and —O-alkyl-(CO)OR₁₄, or two adjacent carbon atoms on the ring form a fused ring with —OCH₂CH₂— or —OCH₂O— that is unsubstituted or substituted by halogen;
R₁₁ each independently represents alkyl, alkenyl, alkynyl, cycloalkyl, cycloalkylalkyl, cycloalkenyl, cycloalkenylalkyl, phenyl, or benzyl, wherein, the “alkyl”, “alkenyl”, or “alkynyl” is each independently unsubstituted or substituted by halogen; the “phenyl” or “benzyl” is each independently unsubstituted or substituted by at least one group selected from halogen, cyano, nitro, alkyl, haloalkyl, alkoxycarbonyl, alkylsulfanyl, alkylsulfonyl, alkoxy, and haloalkoxy;
R₁₂ each independently represents hydrogen, alkyl, alkenyl, alkynyl, alkoxy, alkylsulfonyl, cycloalkyl, cycloalkylalkyl, cycloalkenyl, or cycloalkenylalkyl, or the group N(R₁₂)₂ in —(CO)N(R₁₂)₂ or —(SO₂)N(R₁₂)₂ each independently represents unsubstituted or substituted heterocyclyl with a nitrogen atom at 1-position;
R₁₃ each independently represents alkyl, alkenyl, alkynyl, cycloalkyl, phenyl, or phenyl that is substituted by at least one group selected from halogen, cyano, nitro, alkyl, haloalkyl, alkoxy, haloalkoxy, alkoxycarbonyl, alkylsulfanyl, alkylsulfonyl, and phenoxy that is substituted by at least one group selected from halogen, cyano, nitro, alkyl, haloalkyl, alkoxy, and haloalkoxy;
R₁₄ each independently represents hydrogen, alkyl, haloalkyl, phenyl, or phenyl that is substituted by at least one group selected from halogen, cyano, nitro, alkyl, haloalkyl, alkoxycarbonyl, alkylsulfanyl, alkylsulfonyl, alkoxy, and haloalkoxy;
m represents 0 or 1.

2. The heterocycle-substituted aromatic compound according to claim 1, wherein it is characterized in that,
Y represents halogen, halo C1-C8 alkyl, cyano, nitro, or amino;
preferably, Y represents halogen, halo C1-C6 alkyl, cyano, nitro, or amino.

3. The heterocycle-substituted aromatic compound according to claim 1, wherein it is characterized in that,
R₁, R₂, R₃, R₄, and R₅ each independently represent hydrogen, halogen, hydroxyl, mercapto, formyl, hydroxyl C1-C8 alkyl, nitro, cyano, C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, —OR₁₁, —SR₁₁, —(SO)R₁₁, —(SO₂)R₁₁, —(SO₂)OR₁₁, —O(SO₂)R₁₁, —N(R₁₂)₂, phenyl, or benzyl, wherein,
the “C1-C8 alkyl”, “C2-C8 alkenyl”, “C2-C8 alkynyl”, “C3-C8 cycloalkyl”, “C3-C8 cycloalkyl C1-C8 alkyl”, “C3-C8 cycloalkenyl”, or “C3-C8 cycloalkenyl C1-C8 alkyl” is each independently unsubstituted or substituted by halogen; the “phenyl” or “benzyl” is each independently unsubstituted or substituted by at least one group selected from halogen, cyano, nitro, C1-C8 alkyl, halo C1-C8 alkyl, C1-C8 alkoxycarbonyl, C1-C8 alkylsulfanyl, C1-C8 alkylsulfonyl, C1-C8 alkoxy, and halo C1-C8 alkoxy;
preferably, R₁, R₂, R₃, R₄, and R₅ each independently represent hydrogen, halogen, hydroxyl, mercapto, formyl, hydroxyl C1-C6 alkyl, nitro, cyano, C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, —OR₁₁, —SR₁₁, —(SO)R₁₁, —(SO₂)R₁₁, —(SO₂)OR₁₁, —O(SO₂)R₁₁, —N(R₁₂)₂, phenyl, or benzyl, wherein,
the “C1-C6 alkyl”, “C2-C6 alkenyl”, “C2-C6 alkynyl”, “C3-C6 cycloalkyl”, “C3-C6 cycloalkyl C1-C6 alkyl”, “C3-C6 cycloalkenyl”, or “C3-C6 cycloalkenyl C1-C6 alkyl” is each independently unsubstituted or substituted by halogen; the “phenyl” or “benzyl” is each independently unsubstituted or substituted by 1, 2, or 3 groups selected from halogen, cyano, nitro, C1-C6 alkyl, halo C1-C6 alkyl, C1-C6 alkoxycarbonyl, C1-C6 alkylsulfanyl, C1-C6 alkylsulfonyl, C1-C6 alkoxy, and halo C1-C6 alkoxy;
more preferably, R₁, R₂, R₃, R₄, and R₅ each independently represent hydrogen, halogen, hydroxyl, mercapto, formyl, hydroxyl C1-C6 alkyl, nitro, cyano, C1-C6 alkyl, C2-C6 alkenyl, C2-C6 alkynyl, C3-C6 cycloalkyl, C3-C6 cycloalkyl C1-C3 alkyl, C3-C6 cycloalkenyl, C3-C6 cycloalkenyl C1-C3 alkyl, —OR₁₁, —SR₁₁, —(SO)R₁₁, —(SO₂)R₁₁, —(SO₂)OR₁₁, —O(SO₂)R₁₁, —N(R₁₂)₂, phenyl, or benzyl, wherein,
the “C1-C6 alkyl”, “C2-C6 alkenyl”, “C2-C6 alkynyl”, “C3-C6 cycloalkyl”, “C3-C6 cycloalkyl C1-C3 alkyl”, “C3-C6 cycloalkenyl”, or “C3-C6 cycloalkenyl C1-C3 alkyl” is each independently unsubstituted or substituted by halogen; the “phenyl” or “benzyl” is each independently unsubstituted or substituted by 1, 2, or 3 groups selected from halogen, cyano, nitro, C1-C6 alkyl, halo C1-C6 alkyl, C1-C6 alkoxycarbonyl, C1-C6 alkylsulfanyl, C1-C6 alkylsulfonyl, C1-C6 alkoxy, and halo C1-C6 alkoxy.

4. The heterocycle-substituted aromatic compound according to claim 1, wherein it is characterized in that,
X₁, X₂, X₃, and X₄ each independently represent hydrogen, halogen, nitro, cyano, thiocyanato, hydroxyl, mercapto, sulfo, formyl, haloformyl, azido, C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, —PO(OR′)₂, —OR″, —(CO)R″, —SR″, —(SO)R″, —(SO₂)R″, —Si(R″)₃, —O(CO)R″, —O—(SO₂)R″, —S(CO)R″, —(SO₂)OR″, —O(CO)OR″, —(CO)(CO)OR″ 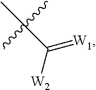
CR′═N—OH, —CR′═N—O—R″, heterocyclyl, heterocyclyl C1-C8 alkyl, aryl, aryl C1-C8 alkyl, amino, amino C1-C8 alkyl, aminocarbonyl C1-C8 alkyl, aminocarbonyloxy C1-C8 alkyl, aminothiocarbonyloxy C1-C8 alkyl, aminosulfonyl, or aminosulfonyloxy C1-C8 alkyl, wherein,
the “C1-C8 alkyl”, “C2-C8 alkenyl”, or “C2-C8 alkynyl” is each independently unsubstituted or substituted by at least one group selected from halogen, cyano, hydroxyl, mercapto, carboxyl, —OR″, —(CO)R″, —SR″, —(SO₂)R″, —O(CO)H, —O(CO)R″, —O—(SO₂)R″, —(CO)OR″, —O(CO)OR″, —O(CO)(CO)OH, —O(CO)(CO)OR″, —O—(C1-C8 alkyl)-(CO)OH, and —O—(C1-C8 alkyl)-(CO)OR″;
the “C3-C8 cycloalkyl”, “C3-C8 cycloalkyl C1-C8 alkyl”, “C3-C8 cycloalkenyl”, “C3-C8 cycloalkenyl C1-C8 alkyl”, “heterocyclyl”, “heterocyclyl C1-C8 alkyl”, “aryl”, or “aryl C1-C8 alkyl” is each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₄, —SR₁₄, —(CO)OR₁₄, —(SO₂)R₁₄, —N(R₁₄)₂, and —O—(C1-C8 alkyl)-(CO)OR₁₄, or two adjacent carbon atoms on the ring form a fused ring with —OCH₂CH₂— or —OCH₂O— that is unsubstituted or substituted by halogen;
the “amino”, “amino C1-C8 alkyl”, “aminocarbonyl C1-C8 alkyl”, “aminocarbonyloxy C1-C8 alkyl”, “aminothiocarbonyloxy C1-C8 alkyl”, “aminosulfonyl”, or “aminosulfonyloxy C1-C8 alkyl” is each independently unsubstituted or substituted by one or two groups selected from —R₁₁, —OR₁₁, —(CO)R₁₁, —(CO)OR₁₁, —C1-C8 alkyl)-(CO)OR₁₁, —(SO₂)R₁₁, —(SO₂)OR₁₁, —C1-C8 alkyl)-(SO₂)R₁₁, —(CO)N(R₁₂)₂, and —(SO₂)N(R₁₂)₂;
R′ each independently represents hydrogen, halogen, C1-C8 alkoxy, C1-C8 alkoxy C1-C8 alkyl, C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, aryl, aryl C1-C8 alkyl, heterocyclyl, or heterocyclyl C1-C8 alkyl, wherein, the “C1-C8 alkyl”, “C2-C8 alkenyl”, or “C2-C8 alkynyl” is each independently unsubstituted or substituted by halogen; the “C3-C8 cycloalkyl”, “C3-C8 cycloalkyl C1-C8 alkyl”, “C3-C8 cycloalkenyl”, “C3-C8 cycloalkenyl C1-C8 alkyl”, “aryl”, “aryl C1-C8 alkyl”, “heterocyclyl”, or “heterocyclyl C1-C8 alkyl” is each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₄, —SR₁₄, —(CO)OR₁₄, —(SO₂)R₁₄, —N(R₁₄)₂, and —O—(C1-C8 alkyl)-(CO)OR₁₄, or two adjacent carbon atoms on the ring form a fused ring with —OCH₂CH₂— or —OCH₂O— that is unsubstituted or substituted by halogen;
R″ each independently represents C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, aryl, aryl C1-C8 alkyl, aryl C2-C8 alkenyl, heterocyclyl, heterocyclyl C1-C8 alkyl, or heterocyclyl C2-C8 alkenyl, wherein, the “C1-C8 alkyl”, “C2-C8 alkenyl”, or “C2-C8 alkynyl” is each independently unsubstituted or substituted by at least one group selected from halogen, cyano, tri-C1-C8 alkylsilyl, —OR₁₃, —SR₁₃, —O(CO)R₁₃, —(CO)R₁₃, —(CO)OR₁₃, and O(CO)OR₁₃; the “C3-C8 cycloalkyl”, “C3-C8 cycloalkyl C1-C8 alkyl”, “C3-C8 cycloalkenyl”, “C3-C8 cycloalkenyl C1-C8 alkyl”, “aryl”, “aryl C1-C8 alkyl”, “aryl C2-C8 alkenyl”, “heterocyclyl”, “heterocyclyl C1-C8 alkyl”, or “heterocyclyl C2-C8 alkenyl” is each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₄, —SR₁₄, —(CO)OR₁₄, —(SO₂)R₁₄, —N(R₁₄)₂, and —O—(C1-C8 alkyl)-(CO)OR₁₄, or two adjacent carbon atoms on the ring form a fused ring with —OCH₂CH₂— or —OCH₂O— that is unsubstituted or substituted by halogen;
preferably, X₁, X₂, X₃, and X₄ each independently represent hydrogen, halogen, nitro, cyano, thiocyanato, hydroxyl, mercapto, sulfo, formyl, haloformyl, azido, C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, —PO(OR′)₂, —OR″, —(CO)R″, —SR″, —(SO)R″, —(SO₂)R″, —Si(R″)₃, —O(CO)R″, —O—(SO₂)R″, —S(CO)R″, —(SO₂)OR″, —O(CO)OR″, —(CO)(CO)OR″, 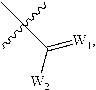
—CR′═N—OH, —CR′═N—O—R″, heterocyclyl, heterocyclyl C1-C6 alkyl, aryl, aryl C1-C6 alkyl, amino, amino C1-C6 alkyl, aminocarbonyl C1-C6 alkyl, aminocarbonyloxy C1-C6 alkyl, aminothiocarbonyloxy C1-C6 alkyl, aminosulfonyl, or aminosulfonyloxy C1-C6 alkyl, wherein,
the “C1-C6 alkyl”, “C2-C6 alkenyl”, or “C2-C6 alkynyl” is each independently unsubstituted or substituted by 1, 2, or 3 groups selected from halogen, cyano, hydroxyl, mercapto, carboxyl, —OR″, —(CO)R″, —SR″, —(SO₂)R″, —O(CO)H, —O(CO)R″, —O—(SO₂)R″, —(CO)OR″, —O(CO)OR″, —O(CO)(CO)OH, —O(CO)(CO)OR″, —O—(C1-C6 alkyl)-(CO)OH, and —O—(C1-C6 alkyl)-(CO)OR″;
the “C3-C6 cycloalkyl”, “C3-C6 cycloalkyl C1-C6 alkyl”, “C3-C6 cycloalkenyl”, “C3-C6 cycloalkenyl C1-C6 alkyl”, “heterocyclyl”, “heterocyclyl C1-C6 alkyl”, “aryl”, or “aryl C1-C6 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C6 alkyl)-(CO)OR₁₄, or two adjacent carbon atoms on the ring form a fused ring with —OCH₂CH₂— or —OCH₂O— that is unsubstituted or substituted by halogen;
the “amino”, “amino C1-C6 alkyl”, “aminocarbonyl C1-C6 alkyl”, “aminocarbonyloxy C1-C6 alkyl”, “aminothiocarbonyloxy C1-C6 alkyl”, “aminosulfonyl”, or “aminosulfonyloxy C1-C6 alkyl” is each independently unsubstituted or substituted by one or two groups selected from —R₁₁, —OR₁₁, —(CO)R₁₁, —(CO)OR₁₁, —C1-C6 alkyl)-(CO)OR₁₁, —(SO₂)R₁₁, —(SO₂)OR₁₁, —C1-C6 alkyl)-(SO₂)R₁₁, —(CO)N(R₁₂)₂, and —(SO₂)N(R₁₂)₂;
R′ each independently represents hydrogen, halogen, C1-C6 alkoxy, C1-C6 alkoxy C1-C6 alkyl, C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, aryl, aryl C1-C6 alkyl, heterocyclyl, or heterocyclyl C1-C6 alkyl, wherein, the “C1-C6 alkyl”, “C2-C6 alkenyl”, or “C2-C6 alkynyl” is each independently unsubstituted or substituted by halogen; the “C3-C6 cycloalkyl”, “C3-C6 cycloalkyl C1-C6 alkyl”, “C3-C6 cycloalkenyl”, “C3-C6 cycloalkenyl C1-C6 alkyl”, “aryl”, “aryl C1-C6 alkyl”, “heterocyclyl”, or “heterocyclyl C1-C6 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C6 alkyl)-(CO)OR₁₄, or two adjacent carbon atoms on the ring form a fused ring with —OCH₂CH₂— or —OCH₂O— that is unsubstituted or substituted by halogen;
R″ each independently represents C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, aryl, aryl C1-C6 alkyl, aryl C2-C6 alkenyl, heterocyclyl, heterocyclyl C1-C6 alkyl, or heterocyclyl C2-C6 alkenyl, wherein, the “C1-C6 alkyl”, “C2-C6 alkenyl”, or “C2-C6 alkynyl” is each independently unsubstituted or substituted by 1, 2, or 3 groups selected from halogen, cyano, tri-C1-C6 alkylsilyl, —OR₁₃, —SR₁₃, —O(CO)R₁₃, —(CO)R₁₃, —(CO)OR₁₃, and —O(CO)OR₁₃;
the “C3-C6 cycloalkyl”, “C3-C6 cycloalkyl C1-C6 alkyl”, “C3-C6 cycloalkenyl”, “C3-C6 cycloalkenyl C1-C6 alkyl”, “aryl”, “aryl C1-C6 alkyl”, “aryl C2-C6 alkenyl”, “heterocyclyl”, “heterocyclyl C1-C6 alkyl”, or “heterocyclyl C2-C6 alkenyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C6 alkyl)-(CO)OR₁₄, or two adjacent carbon atoms on the ring form a fused ring with —OCH₂CH₂— or —OCH₂O— that is unsubstituted or substituted by halogen;
more preferably, X₁, X₂, X₃, and X₄ each independently represent hydrogen, halogen, nitro, cyano, thiocyanato, hydroxyl, mercapto, sulfo, formyl, haloformyl, azido, C1-C6 alkyl, C2-C6 alkenyl, C2-C6 alkynyl, C3-C6 cycloalkyl, C3-C6 cycloalkyl C1-C3 alkyl, C3-C6 cycloalkenyl, C3-C6 cycloalkenyl C1-C3 alkyl, —PO(OR′)₂, —OR″, —(CO)R″, —SR″, —(SO)R″, —(SO₂)R″, —Si(R″)₃, —O(CO)R″, —O—(SO₂)R″, —S(CO)R″, —(SO₂)OR″, —O(CO)OR″, —(CO)(CO)OR″, 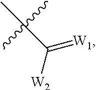
—CR′═N—OH, —CR′═N—O—R″, heterocyclyl, heterocyclyl C1-C3 alkyl, aryl, aryl C1-C3 alkyl, amino, amino C1-C3 alkyl, aminocarbonyl C1-C3 alkyl, aminocarbonyloxy C1-C3 alkyl, aminothiocarbonyloxy C1-C3 alkyl, aminosulfonyl, or aminosulfonyloxy C1-C3 alkyl, wherein,
the “C1-C6 alkyl”, “C2-C6 alkenyl”, or “C2-C6 alkynyl” is each independently unsubstituted or substituted by 1, 2, or 3 groups selected from halogen, cyano, hydroxyl, mercapto, carboxyl, —OR″, —(CO)R″, —SR″, —(SO₂)R″, —O(CO)H, —O(CO)R″, —O—(SO₂)R″, —(CO)OR″, —O(CO)OR″, —O(CO)(CO)OH, —O(CO)(CO)OR″, —O—(C1-C3 alkyl)-(CO)OH, and —O—(C1-C3 alkyl)-(CO)OR″;
the “C3-C6 cycloalkyl”, “C3-C6 cycloalkyl C1-C3 alkyl”, “C3-C6 cycloalkenyl”, “C3-C6 cycloalkenyl C1-C3 alkyl”, “heterocyclyl”, “heterocyclyl C1-C3 alkyl”, “aryl”, or “aryl C1-C3 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C3 alkyl)-(CO)OR₁₄, or two adjacent carbon atoms on the ring form a fused ring with —OCH₂CH₂— or —OCH₂O— that is unsubstituted or substituted by halogen;
the “amino”, “amino C1-C3 alkyl”, “aminocarbonyl C1-C3 alkyl”, “aminocarbonyloxy C1-C3 alkyl”, “aminothiocarbonyloxy C1-C3 alkyl”, “aminosulfonyl”, or “aminosulfonyloxy C1-C3 alkyl” is each independently unsubstituted or substituted by one or two groups selected from —R₁₁, —OR₁₁, —(CO)R₁₁, —(CO)OR₁₁, —C1-C3 alkyl)-(CO)OR₁₁, —(SO₂)R₁₁, —(SO₂)OR₁₁, —C1-C3 alkyl)-(SO₂)R₁₁, —(CO)N(R₁₂)₂, and —(SO₂)N(R₁₂)₂;
R′ each independently represents hydrogen, halogen, C1-C6 alkoxy, C1-C6 alkoxy C1-C3 alkyl, C1-C6 alkyl, C2-C6 alkenyl, C2-C6 alkynyl, C3-C6 cycloalkyl, C3-C6 cycloalkyl C1-C3 alkyl, C3-C6 cycloalkenyl, C3-C6 cycloalkenyl C1-C3 alkyl, aryl, aryl C1-C3 alkyl, heterocyclyl, or heterocyclyl C1-C3 alkyl, wherein, the “C1-C6 alkyl”, “C2-C6 alkenyl”, or “C2-C6 alkynyl” is each independently unsubstituted or substituted by halogen; the “C3-C6 cycloalkyl”, “C3-C6 cycloalkyl C1-C3 alkyl”, “C3-C6 cycloalkenyl”, “C3-C6 cycloalkenyl C1-C3 alkyl”, “aryl”, “aryl C1-C3 alkyl”, “heterocyclyl”, or “heterocyclyl C1-C3 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C3 alkyl)-(CO)OR₁₄, or two adjacent carbon atoms on the ring form a fused ring with —OCH₂CH₂— or —OCH₂O— that is unsubstituted or substituted by halogen;
R″ each independently represents C1-C6 alkyl, C2-C6 alkenyl, C2-C6 alkynyl, C3-C6 cycloalkyl, C3-C6 cycloalkyl C1-C3 alkyl, C3-C6 cycloalkenyl, C3-C6 cycloalkenyl C1-C3 alkyl, aryl, aryl C1-C3 alkyl, aryl C2-C3 alkenyl, heterocyclyl, heterocyclyl C1-C3 alkyl, or heterocyclyl C2-C3 alkenyl, wherein, the “C1-C6 alkyl”, “C2-C6 alkenyl”, or “C2-C6 alkynyl” is each independently unsubstituted or substituted by 1, 2, or 3 groups selected from halogen, cyano, tri-C1-C6 alkylsilyl, —OR₁₃, —SR₁₃, —O(CO)R₁₃, —(CO)R₁₃, —(CO)OR₁₃, and —O(CO)OR₁₃;
the “C3-C6 cycloalkyl”, “C3-C6 cycloalkyl C1-C3 alkyl”, “C3-C6 cycloalkenyl”, “C3-C6 cycloalkenyl C1-C3 alkyl”, “aryl”, “aryl C1-C3 alkyl”, “aryl C2-C3 alkenyl”, “heterocyclyl”, “heterocyclyl C1-C3 alkyl”, or “heterocyclyl C2-C3 alkenyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C3 alkyl)-(CO)OR₁₄, or two adjacent carbon atoms on the ring form a fused ring with —OCH₂CH₂— or —OCH₂O— that is unsubstituted or substituted by halogen.

5. The heterocycle-substituted aromatic compound according to claim 1, wherein it is characterized in that,
W₃ each independently represents hydrogen, C1-C8 alkyl, C2-C8 alkenyl, C2-C8 alkynyl, C3-C8 cycloalkyl, C3-C8 cycloalkenyl, heterocyclyl, aryl, 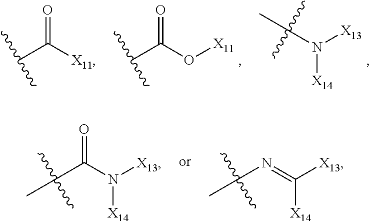
wherein, the “C1-C8 alkyl”, “C2-C8 alkenyl”, or “C2-C8 alkynyl” is each independently unsubstituted or substituted by at least one group selected from halogen, cyano, nitro, C3-C8 cycloalkyl, tri-C1-C8 alkylsilyl, C3-C8 cycloalkenyl, heterocyclyl, aryl, 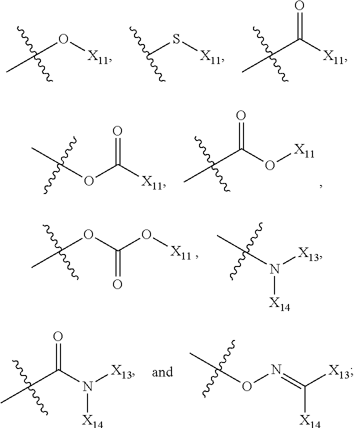
the “C3-C8 cycloalkyl”, “C3-C8 cycloalkenyl”, “heterocyclyl”, or “aryl” is each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₄, —SR₁₄, —(CO)OR₁₄, —(SO₂)R₁₄, —N(R₁₄)₂, and —O—(C1-C8 alkyl)-(CO)OR₁₄, or two adjacent carbon atoms on the ring form a fused ring with —OCH₂CH₂— or —OCH₂O— that is unsubstituted or substituted by halogen;
W₄ and W₅ each independently represent hydrogen, C1-C8 alkyl, C2-C8 alkenyl, C2-C8 alkynyl, C3-C8 cycloalkyl, C3-C8 cycloalkenyl, heterocyclyl, aryl, 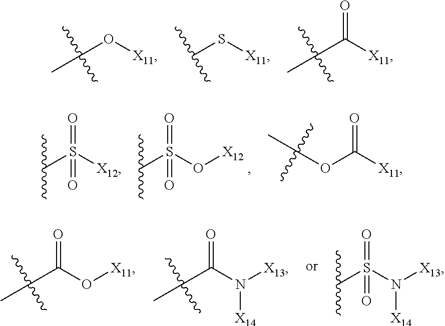
wherein, the “C1-C8 alkyl”, “C2-C8 alkenyl”, or “C2-C8 alkynyl” is each independently unsubstituted or substituted by at least one group selected from halogen, cyano, nitro, C3-C8 cycloalkyl, tri-C1-C8 alkylsilyl, C3-C8 cycloalkenyl, heterocyclyl, aryl, 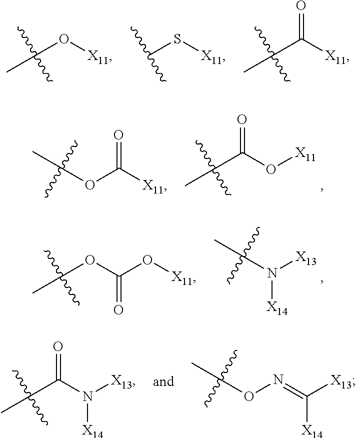
the “C3-C8 cycloalkyl”, “C3-C8 cycloalkenyl”, “heterocyclyl”, or “aryl” is each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₄, —SR₁₄, —(CO)OR₁₄, —(SO₂)R₁₄, —N(R₁₄)₂, and —O—(C1-C8 alkyl)-(CO)OR₁₄, or two adjacent carbon atoms on the ring form a fused ring with —OCH₂CH₂— or —OCH₂O— that is unsubstituted or substituted by halogen;
or NW₄W₅ represents 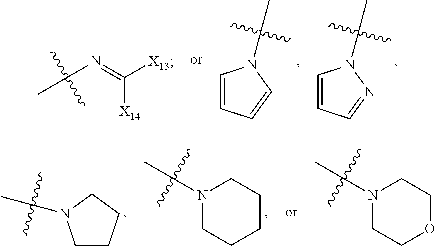
which is unsubstituted or substituted by at least one group selected from oxo, C1-C8 alkyl, and C1-C8 alkoxycarbonyl;
X₁₁ each independently represents hydrogen, C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, heterocyclyl, heterocyclyl C1-C8 alkyl, aryl, or aryl C1-C8 alkyl, wherein, the “C3-C8 cycloalkyl”, “C3-C8 cycloalkyl C1-C8 alkyl”, “C3-C8 cycloalkenyl”, “C3-C8 cycloalkenyl C1-C8 alkyl”, “heterocyclyl”, “heterocyclyl C1-C8 alkyl”, “aryl”, or “aryl C1-C8 alkyl” is each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₄, —SR₁₄, —(CO)OR₁₄, —(SO₂)R₁₄, —N(R₁₄)₂, and —O—(C1-C8 alkyl)-(CO)OR₁₄, or two adjacent carbon atoms on the ring form a fused ring with —OCH₂CH₂— or —OCH₂O— that is unsubstituted or substituted by halogen;
X₁₂ each independently represents C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, heterocyclyl, heterocyclyl C1-C8 alkyl, aryl, or aryl C1-C8 alkyl, wherein, the “C3-C8 cycloalkyl”, “C3-C8 cycloalkyl C1-C8 alkyl”, “C3-C8 cycloalkenyl”, “C3-C8 cycloalkenyl C1-C8 alkyl”, “heterocyclyl”, “heterocyclyl C1-C8 alkyl”, “aryl”, or “aryl C1-C8 alkyl” is each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₄, —SR₁₄, —(CO)OR₁₄, —(SO₂)R₁₄, —N(R₁₄)₂, and —O—(C1-C8 alkyl)-(CO)OR₁₄, or two adjacent carbon atoms on the ring form a fused ring with —OCH₂CH₂— or —OCH₂O— that is unsubstituted or substituted by halogen;
X₁₃ and X₁₄ each independently represent hydrogen, halogen, cyano, C1-C8 alkoxy, C1-C8 alkoxy C1-C8 alkyl, C1-C8 alkylcarbonyl, C1-C8 alkoxycarbonyl, C1-C8 alkylsulfonyl, C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, aryl, aryl C1-C8 alkyl, heterocyclyl, or heterocyclyl C1-C8 alkyl, or the group CX₁₃X₁₄ together forms a 5-8 membered carbocycle or oxygen-, sulfur-, or nitrogen-containing heterocycle, or the group NX₁₃X₁₄ together forms 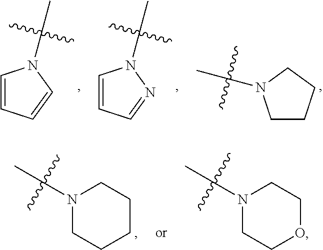
wherein, the “C1-C8 alkyl”, “C2-C8 alkenyl”, or “C2-C8 alkynyl” is each independently unsubstituted or substituted by halogen; the “C3-C8 cycloalkyl”, “C3-C8 cycloalkyl C1-C8 alkyl”, “C3-C8 cycloalkenyl”, “C3-C8 cycloalkenyl C1-C8 alkyl”, “aryl”, “aryl C1-C8 alkyl”, “heterocyclyl”, or “heterocyclyl C1-C8 alkyl” is each independently unsubstituted or substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₄, —SR₁₄, —(CO)OR₁₄, —(SO₂)R₁₄, —N(R₁₄)₂, and —O—(C1-C8 alkyl)-(CO)OR₁₄, or two adjacent carbon atoms on the ring form a fused ring with —OCH₂CH₂— or —OCH₂O— that is unsubstituted or substituted by halogen; the “5-8 membered carbocycle or oxygen-, sulfur-, or nitrogen-containing heterocycle” is unsubstituted or substituted by at least one group selected from C1-C8 alkyl, C1-C8 alkoxycarbonyl, and benzyl, or forms a fused ring structure with aryl or heterocyclyl; the 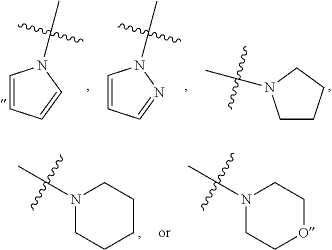
is unsubstituted or substituted by at least one group selected from oxo, C1-C8 alkyl, and C1-C8 alkoxycarbonyl;
preferably, W₃ each independently represents hydrogen, C1-C6 alkyl, C2-C6 alkenyl, C2-C6 alkynyl, C3-C6 cycloalkyl, C3-C6 cycloalkenyl, heterocyclyl, aryl, 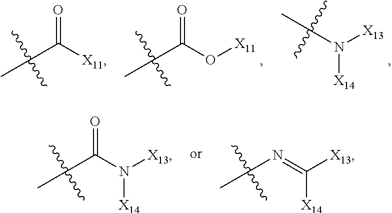
wherein, the “C1-C6 alkyl”, “C2-C6 alkenyl”, or “C2-C6 alkynyl” is each independently unsubstituted or substituted by 1, 2, or 3 groups selected from halogen, cyano, nitro, C3-C6 cycloalkyl, tri-C1-C6 alkylsilyl, C3-C6 cycloalkenyl, heterocyclyl, aryl, 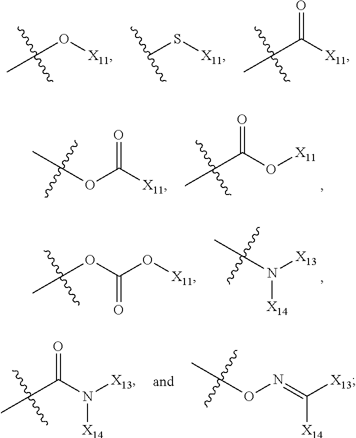
the “C3-C6 cycloalkyl”, “C3-C6 cycloalkenyl”, “heterocyclyl”, or “aryl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C6 alkyl)-(CO)OR₁₄, or two adjacent carbon atoms on the ring form a fused ring with —OCH₂CH₂— or —OCH₂O— that is unsubstituted or substituted by halogen;
W₄ and W₅ each independently represent hydrogen, C1-C6 alkyl, C2-C6 alkenyl, C2-C6 alkynyl, C3-C6 cycloalkyl, C3-C6 cycloalkenyl, heterocyclyl, aryl, 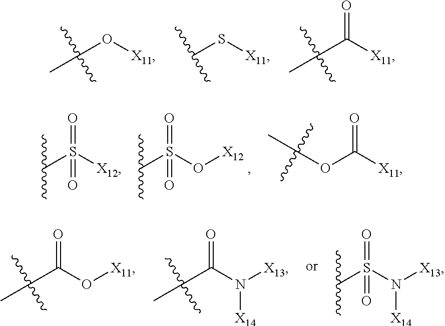
wherein, the “C1-C6 alkyl”, “C2-C6 alkenyl”, or “C2-C6 alkynyl” is each independently unsubstituted or substituted by 1, 2, or 3 groups selected from halogen, cyano, nitro, C3-C6 cycloalkyl, tri-C1-C6 alkylsilyl, C3-C6 cycloalkenyl, heterocyclyl, aryl, 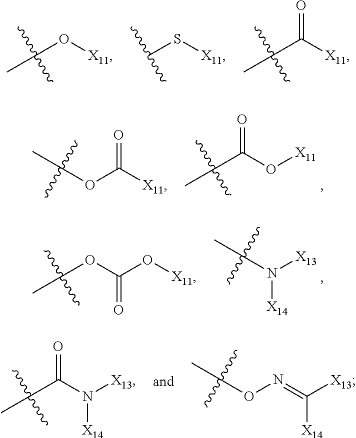
the “C3-C6 cycloalkyl”, “C3-C6 cycloalkenyl”, “heterocyclyl”, or “aryl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C6 alkyl)-(CO)OR₁₄, or two adjacent carbon atoms on the ring form a fused ring with —OCH₂CH₂— or —OCH₂O— that is unsubstituted or substituted by halogen;
or NW₄W₅ represents 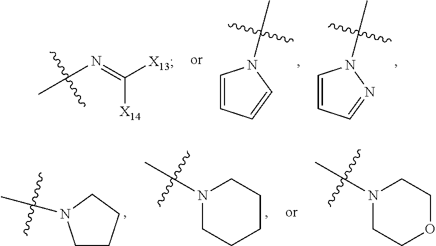
which is unsubstituted or substituted by 1, 2, or 3 groups selected from oxo, C1-C6 alkyl, and C1-C6 alkoxycarbonyl;
X₁₁ each independently represents hydrogen, C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, heterocyclyl, heterocyclyl C1-C6 alkyl, aryl, or aryl C1-C6 alkyl, wherein, the “C3-C6 cycloalkyl”, “C3-C6 cycloalkyl C1-C6 alkyl”, “C3-C6 cycloalkenyl”, “C3-C6 cycloalkenyl C1-C6 alkyl”, “heterocyclyl”, “heterocyclyl C1-C6 alkyl”, “aryl”, or “aryl C1-C6 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C6 alkyl)-(CO)OR₁₄, or two adjacent carbon atoms on the ring form a fused ring with —OCH₂CH₂— or —OCH₂O— that is unsubstituted or substituted by halogen;
X₁₂ each independently represents C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, heterocyclyl, heterocyclyl C1-C6 alkyl, aryl, or aryl C1-C6 alkyl, wherein, the “C3-C6 cycloalkyl”, “C3-C6 cycloalkyl C1-C6 alkyl”, “C3-C6 cycloalkenyl”, “C3-C6 cycloalkenyl C1-C6 alkyl”, “heterocyclyl”, “heterocyclyl C1-C6 alkyl”, “aryl”, or “aryl C1-C6 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C6 alkyl)-(CO)OR₁₄, or two adjacent carbon atoms on the ring form a fused ring with —OCH₂CH₂— or —OCH₂O— that is unsubstituted or substituted by halogen;
X₁₃ and X₁₄ each independently represent hydrogen, halogen, cyano, C1-C6 alkoxy, C1-C6 alkoxy C1-C6 alkyl, C1-C6 alkylcarbonyl, C1-C6 alkoxycarbonyl, C1-C6 alkylsulfonyl, C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, aryl, aryl C1-C6 alkyl, heterocyclyl, or heterocyclyl C1-C6 alkyl, or the group CX₁₃X₁₄ together forms a 5-8 membered saturated carbocycle, 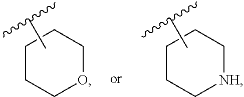
or the group NX₁₃X₁₄ together forms 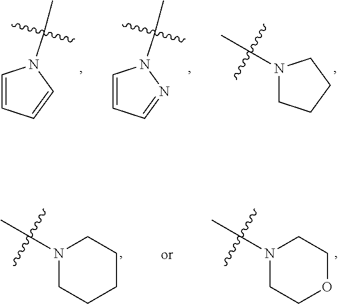
wherein, the “C1-C6 alkyl”, “C2-C6 alkenyl”, or “C2-C6 alkynyl” is each independently unsubstituted or substituted by halogen; the “C3-C6 cycloalkyl”, “C3-C6 cycloalkyl C1-C6 alkyl”, “C3-C6 cycloalkenyl”, “C3-C6 cycloalkenyl C1-C6 alkyl”, “aryl”, “aryl C1-C6 alkyl”, “heterocyclyl”, or “heterocyclyl C1-C6 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C6 alkyl)-(CO)OR₁₄, or two adjacent carbon atoms on the ring form a fused ring with —OCH₂CH₂— or —OCH₂O— that is unsubstituted or substituted by halogen; the “5-8 membered saturated carbocycle, 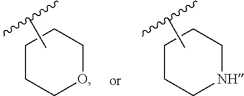
is unsubstituted or substituted by 1, 2, or 3 groups selected from C1-C6 alkyl, C1-C6 alkoxycarbonyl, and benzyl, or forms a fused ring with aryl or heterocyclyl; the, 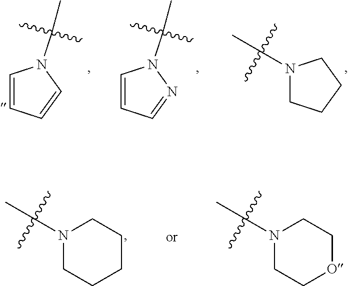
is unsubstituted or substituted by 1, 2, or 3 groups selected from oxo, C1-C6 alkyl, and C1-C6 alkoxycarbonyl;
more preferably, W₃ each independently represents hydrogen, C1-C6 alkyl, C2-C6 alkenyl, C2-C6 alkynyl, C3-C6 cycloalkyl, C3-C6 cycloalkenyl, heterocyclyl, aryl, 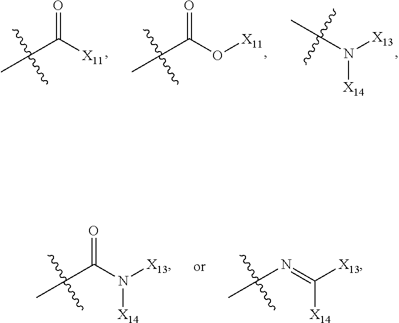
wherein, the “C1-C6 alkyl”, “C2-C6 alkenyl”, or “C2-C6 alkynyl” is each independently unsubstituted or substituted by 1, 2, or 3 groups selected from halogen, cyano, nitro, C3-C6 cycloalkyl, tri-C1-C6 alkylsilyl, C3-C6 cycloalkenyl, heterocyclyl, aryl, 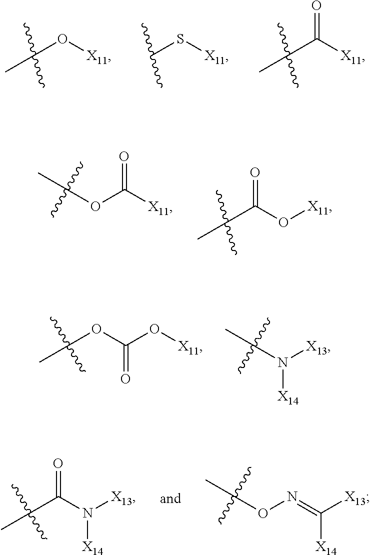
the “C3-C6 cycloalkyl”, “C3-C6 cycloalkenyl”, “heterocyclyl”, or “aryl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3—C6 cycloalkyl, —OR₁₄, —SR₁₄, —(CO)OR₁₄, —(SO₂)R₁₄, —N(R₁₄)₂, and —O—(C1-C3 alkyl)-(CO)OR₁₄, or two adjacent carbon atoms on the ring form a fused ring with —OCH₂CH₂— or —OCH₂O— that is unsubstituted or substituted by halogen;
W₄ and W₅ each independently represent hydrogen, C1-C6 alkyl, C2-C6 alkenyl, C2-C6 alkynyl, C3-C6 cycloalkyl, C3-C6 cycloalkenyl, heterocyclyl, aryl, 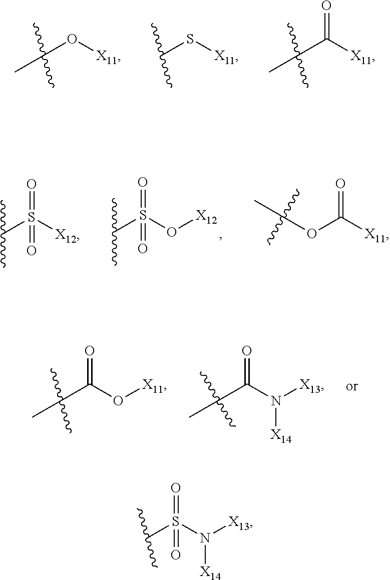
wherein, the “C1-C6 alkyl”, “C2-C6 alkenyl”, or “C2-C6 alkynyl” is each independently unsubstituted or substituted by 1, 2, or 3 groups selected from halogen, cyano, nitro, C3-C6 cycloalkyl, tri-C1-C6 alkylsilyl, C3-C6 cycloalkenyl, heterocyclyl, aryl, 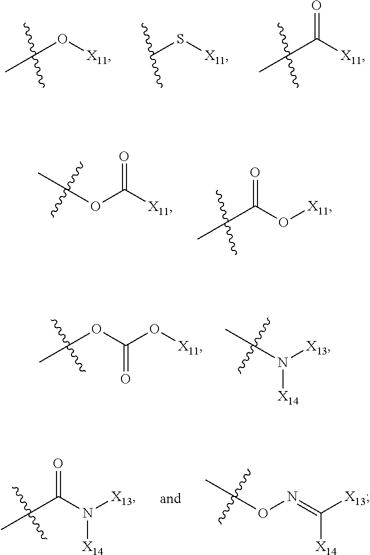
the “C3-C6 cycloalkyl”, “C3-C6 cycloalkenyl”, “heterocyclyl”, or “aryl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C3 alkyl)-(CO)OR₁₄, or two adjacent carbon atoms on the ring form a fused ring with —OCH₂CH₂— or —OCH₂O— that is unsubstituted or substituted by halogen;
or NW₄W₅ represents 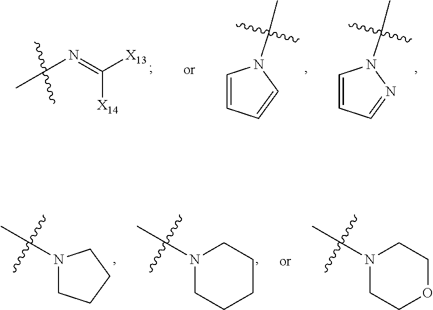
which is unsubstituted or substituted by 1, 2, or 3 groups selected from oxo, C1-C6 alkyl, and C1-C6 alkoxycarbonyl;
X₁₁ each independently represents hydrogen, C1-C6 alkyl, C2-C6 alkenyl, C2-C6 alkynyl, C3-C6 cycloalkyl, C3-C6 cycloalkyl C1-C3 alkyl, C3-C6 cycloalkenyl, C3-C6 cycloalkenyl C1-C3 alkyl, heterocyclyl, heterocyclyl C1-C3 alkyl, aryl, or aryl C1-C3 alkyl, wherein, the “C3-C6 cycloalkyl”, “C3-C6 cycloalkyl C1-C3 alkyl”, “C3-C6 cycloalkenyl”, “C3-C6 cycloalkenyl C1-C3 alkyl”, “heterocyclyl”, “heterocyclyl C1-C3 alkyl”, “aryl”, or “aryl C1-C3 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C3 alkyl)-(CO)OR₁₄, or two adjacent carbon atoms on the ring form a fused ring with —OCH₂CH₂— or —OCH₂O— that is unsubstituted or substituted by halogen;
X₁₂ each independently represents C1-C6 alkyl, C2-C6 alkenyl, C2-C6 alkynyl, C3-C6 cycloalkyl, C3-C6 cycloalkyl C1-C3 alkyl, C3-C6 cycloalkenyl, C3-C6 cycloalkenyl C1-C3 alkyl, heterocyclyl, heterocyclyl C1-C3 alkyl, aryl, or aryl C1-C3 alkyl, wherein, the “C3-C6 cycloalkyl”, “C3-C6 cycloalkyl C1-C3 alkyl”, “C3-C6 cycloalkenyl”, “C3-C6 cycloalkenyl C1-C3 alkyl”, “heterocyclyl”, “heterocyclyl C1-C3 alkyl”, “aryl”, or “aryl C1-C3 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₄, —SR₁₄, —(CO)OR₁₄, —(SO₂)R₁₄, —N(R₁₄)₂, and —O—(C1-C3 alkyl)-(CO)OR₁₄, or two adjacent carbon atoms on the ring form a fused ring with —OCH₂CH₂— or —OCH₂O— that is unsubstituted or substituted by halogen;
X₁₃ and X₁₄ each independently represent hydrogen, halogen, cyano, C1-C6 alkoxy, C1-C6 alkoxy C1-C3 alkyl, C1-C6 alkylcarbonyl, C1-C6 alkoxycarbonyl, C1-C6 alkylsulfonyl, C1-C6 alkyl, C2-C6 alkenyl, C2-C6 alkynyl, C3-C6 cycloalkyl, C3-C6 cycloalkyl C1-C3 alkyl, C3-C6 cycloalkenyl, C3-C6 cycloalkenyl C1-C3 alkyl, aryl, aryl C1-C3 alkyl, heterocyclyl, or heterocyclyl C1-C3 alkyl, or the group CX₁₃X₁₄ together forms a 5-8 membered saturated carbocycle, 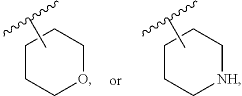
or the group NX₁₃X₁₄ together forms 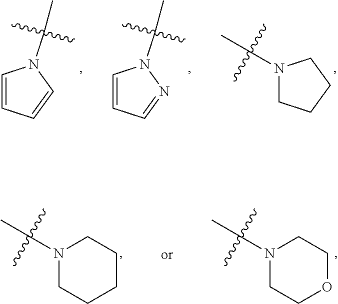
wherein, the “C1-C6 alkyl”, “C2-C6 alkenyl”, or “C2-C6 alkynyl” is each independently unsubstituted or substituted by halogen; the “C3-C6 cycloalkyl”, “C3-C6 cycloalkyl C1-C3 alkyl”, “C3-C6 cycloalkenyl”, “C3-C6 cycloalkenyl C1-C3 alkyl”, “aryl”, “aryl C1-C3 alkyl”, “heterocyclyl”, or “heterocyclyl C1-C3 alkyl” is each independently unsubstituted or substituted by 1, 2, or 3 groups selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3—C6 cycloalkyl, —OR₁₄, —SR₁₄, —(CO)OR₁₄, —(SO₂)R₁₄, —N(R₁₄)₂, and —O—(C1-C3 alkyl)-(CO)OR₁₄, or two adjacent carbon atoms on the ring form a fused ring with —OCH₂CH₂— or —OCH₂O— that is unsubstituted or substituted by halogen; the “5-8 membered saturated carbocycle, 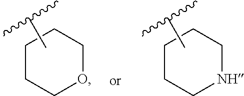
is unsubstituted or substituted by 1, 2, or 3 groups selected from C1-C6 alkyl, C1-C6 alkoxycarbonyl, and benzyl, or forms a fused ring with phenyl or thienyl; the 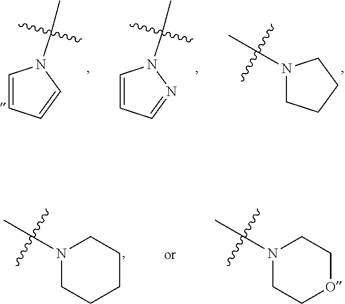
is unsubstituted or substituted by 1, 2, or 3 groups selected from oxo, C1-C6 alkyl, and C1-C6 alkoxycarbonyl.

6. The heterocycle-substituted aromatic compound according to claim 1, wherein it is characterized in that,
R₁₁ each independently represents C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, phenyl, or benzyl, wherein, the “C1-C8 alkyl”, “C2-C8 alkenyl”, or “C2-C8 alkynyl” is each independently unsubstituted or substituted by halogen; the “phenyl” or “benzyl” is each independently unsubstituted or substituted by at least one group selected from halogen, cyano, nitro, C1-C8 alkyl, halo C1-C8 alkyl, C1-C8 alkoxycarbonyl, C1-C8 alkylsulfanyl, C1-C8 alkylsulfonyl, C1-C8 alkoxy, and halo C1-C8 alkoxy;
R₁₂ each independently represents hydrogen, C1-C8 alkyl, C2-C8 alkenyl, C2-C8 alkynyl, C1-C8 alkoxy, C1-C8 alkylsulfonyl, C3-C8 cycloalkyl, C3-C8 cycloalkyl C1-C8 alkyl, C3-C8 cycloalkenyl, or C3-C8 cycloalkenyl C1-C8 alkyl, or the group N(R₁₂)₂ in —(CO)N(R₁₂)₂ or —(SO₂)N(R₁₂)₂ each independently represents 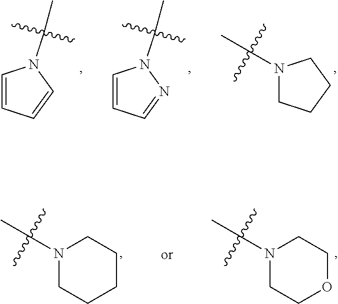 [SMILES: *n1cccc1 ; *n1cccn1 ; *N1CCCC1 ; *N1CCCCC1 ; *N1CCOCC1]
which is unsubstituted or substituted by at least one group selected from oxo, C1-C8 alkyl, and C1-C8 alkoxycarbonyl;
R₁₃ each independently represents C1-C8 alkyl, C2-C8 alkenyl, C2-C8 alkynyl, C3-C8 cycloalkyl, phenyl, or phenyl that is substituted by at least one group selected from halogen, cyano, nitro, C1-C8 alkyl, halo C1-C8 alkyl, C1-C8 alkoxy, halo C1-C8 alkoxy, C1-C8 alkoxycarbonyl, C1-C8 alkylsulfanyl, C1-C8 alkylsulfonyl, and phenoxy that is substituted by at least one group selected from halogen, cyano, nitro, C1-C8 alkyl, halo C1-C8 alkyl, C1-C8 alkoxy, and halo C1-C8 alkoxy;
R₁₄ each independently represents hydrogen, C1-C8 alkyl, halo C1-C8 alkyl, phenyl, or phenyl that is substituted by at least one group selected from halogen, cyano, nitro, C1-C8 alkyl, halo C1-C8 alkyl, C1-C8 alkoxycarbonyl, C1-C8 alkylsulfanyl, C1-C8 alkylsulfonyl, C1-C8 alkoxy, and halo C1-C8 alkoxy;
preferably, R₁₁ each independently represents C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, phenyl, or benzyl, wherein, the “C1-C6 alkyl”, “C2-C6 alkenyl”, or “C2-C6 alkynyl” is each independently unsubstituted or substituted by halogen; the “phenyl” or “benzyl” is each independently unsubstituted or substituted by 1, 2, or 3 groups selected from halogen, cyano, nitro, C1-C6 alkyl, halo C1-C6 alkyl, C1-C6 alkoxycarbonyl, C1-C6 alkylsulfanyl, C1-C6 alkylsulfonyl, C1-C6 alkoxy, and halo C1-C6 alkoxy;
R₁₂ each independently represents hydrogen, C1-C6 alkyl, C2-C6 alkenyl, C2-C6 alkynyl, C1-C6 alkoxy, C1-C6 alkylsulfonyl, C3-C6 cycloalkyl, C3-C6 cycloalkyl C1-C6 alkyl, C3-C6 cycloalkenyl, or C3-C6 cycloalkenyl C1-C6 alkyl, or the group N(R₁₂)₂ in —(CO N(R₁₂)₂ or —(SO₂)N(R₁₂)₂ each independently represents 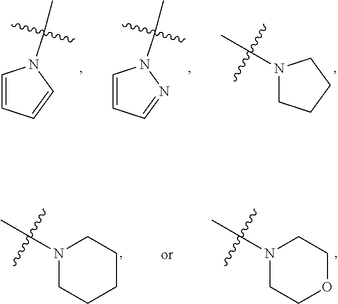 [SMILES: *n1cccc1 ; *n1cccn1 ; *N1CCCC1 ; *N1CCCCC1 ; *N1CCOCC1]
which is unsubstituted or substituted by 1, 2, or 3 groups selected from oxo, C1-C6 alkyl, and C1-C6 alkoxycarbonyl;
R₁₃ each independently represents C1-C6 alkyl, C2-C6 alkenyl, C2-C6 alkynyl, C3-C6 cycloalkyl, phenyl, or phenyl that is substituted by 1, 2, or 3 groups selected from halogen, cyano, nitro, C1-C6 alkyl, halo C1-C6 alkyl, C1-C6 alkoxy, halo C1-C6 alkoxy, C1-C6 alkoxycarbonyl, C1-C6 alkylsulfanyl, C1-C6 alkylsulfonyl, and phenoxy that is substituted by 1, 2, or 3 groups selected from halogen, cyano, nitro, C1-C6 alkyl, halo C1-C6 alkyl, C1-C6 alkoxy, and halo C1-C6 alkoxy;
R₁₄ each independently represents hydrogen, C1-C6 alkyl, halo C1-C6 alkyl, phenyl, or phenyl that is substituted by 1, 2, or 3 groups selected from halogen, cyano, nitro, C1-C6 alkyl, halo C1-C6 alkyl, C1-C6 alkoxycarbonyl, C1-C6 alkylsulfanyl, C1-C6 alkylsulfonyl, C1-C6 alkoxy, and halo C1-C6 alkoxy;
more preferably, R₁₁ each independently represents C1-C6 alkyl, C2-C6 alkenyl, C2-C6 alkynyl, C3-C6 cycloalkyl, C3-C6 cycloalkyl C1-C3 alkyl, C3-C6 cycloalkenyl, C3-C6 cycloalkenyl C1-C3 alkyl, phenyl, or benzyl, wherein, the “C1-C6 alkyl”, “C2-C6 alkenyl”, or “C2-C6 alkynyl” is each independently unsubstituted or substituted by halogen; the “phenyl” or “benzyl” is each independently unsubstituted or substituted by 1, 2, or 3 groups selected from halogen, cyano, nitro, C1-C6 alkyl, halo C1-C6 alkyl, C1-C6 alkoxycarbonyl, C1-C6 alkylsulfanyl, C1-C6 alkylsulfonyl, C1-C6 alkoxy, and halo C1-C6 alkoxy;
R₁₂ each independently represents hydrogen, C1-C6 alkyl, C2-C6 alkenyl, C2-C6 alkynyl, C1-C6 alkoxy, C1-C6 alkylsulfonyl, C3-C6 cycloalkyl, C3-C6 cycloalkyl C1-C3 alkyl, C3-C6 cycloalkenyl, or C3-C6 cycloalkenyl C1-C3 alkyl, or the group N(R₁₂)₂ in —(CO)N(R₁₂)₂ or —(SO₂)N(R₁₂)₂ each independently represents 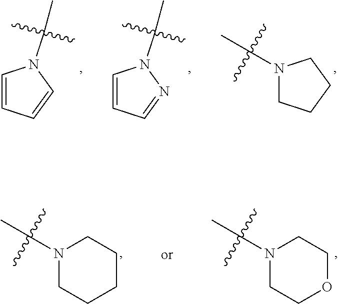 [SMILES: *n1cccc1 ; *n1cccn1 ; *N1CCCC1 ; *N1CCCCC1 ; *N1CCOCC1]
which is unsubstituted or substituted by 1, 2, or 3 groups selected from oxo, C1-C6 alkyl, and C1-C6 alkoxycarbonyl;
R₁₃ each independently represents C1-C6 alkyl, C2-C6 alkenyl, C2-C6 alkynyl, C3-C6 cycloalkyl, phenyl, or phenyl that is substituted by 1, 2, or 3 groups selected from halogen, cyano, nitro, C1-C6 alkyl, halo C1-C6 alkyl, C1-C6 alkoxy, halo C1-C6 alkoxy, C1-C6 alkoxycarbonyl, C1-C6 alkylsulfanyl, C1-C6 alkylsulfonyl, and phenoxy that is substituted by 1, 2, or 3 groups selected from halogen, cyano, nitro, C1-C6 alkyl, halo C1-C6 alkyl, C1-C6 alkoxy, and halo C1-C6 alkoxy;
R₁₄ each independently represents hydrogen, C1-C6 alkyl, halo C1-C6 alkyl, phenyl, or phenyl that is substituted by 1, 2, or 3 groups selected from halogen, cyano, nitro, C1-C6 alkyl, halo C1-C6 alkyl, C1-C6 alkoxycarbonyl, C1-C6 alkylsulfanyl, C1-C6 alkylsulfonyl, C1-C6 alkoxy, and halo C1-C6 alkoxy;
further preferably, the compound is selected from any one in Table 1.

7. A method for preparing the heterocycle-substituted aromatic compound according claim 1, wherein it is characterized in that it comprises the following steps:
subjecting the compound shown in the general formula II and the compound shown in the general formula III to reaction to prepare the compound as shown in the general formula I, and the chemical reaction equation is as follows: 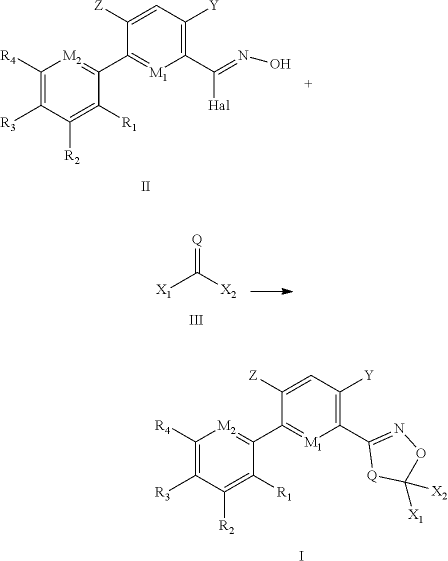
wherein, Hal represents halogen; the definitions of substituents X₁, X₂, R₁, R₂, R₃, R₄, M, M₂, Q, Y, and Z are as described in claim 1;
preferably, the reaction is carried out in the presence of a base and a solvent; more preferably, the base is selected from at least one of nitrogen-containing bases; the solvent is selected from at least one of aromatic hydrocarbons, THF, DMF, DMA, methanol, ethanol, acetonitrile, dichloroethane, DMSO, dioxane, dichloromethane, toluene, and ethyl acetate.

8. A herbicidal composition, wherein it is characterized in that it comprises a herbicidally effective amount of at least one of the heterocycle-substituted aromatic compound according to claim 1, preferably, also comprising a formulation auxiliary.

9. A method for controlling a weed, wherein it is characterized in that it comprises applying a herbicidally effective amount of at least one of the heterocycle-substituted aromatic compound according to claim 1 to a plant or a weedy area.

10. A method for preventing and controlling a weed among a useful crop, comprising applying a herbicidally effective amount of at least one of the heterocycle-substituted aromatic compound according to claim 1.

11. A method for controlling a weed, wherein it is characterized in that it comprises applying a herbicidally effective amount of the herbicidal composition according to claim 8 to a plant or a weedy area.

12. The method according to claim 10, wherein the useful crop is a transgenic crop or a crop treated by a genome editing technique.

13. A method for preventing and controlling a weed among a useful crop, comprising applying a herbicidally effective amount of the herbicidal composition according to claim 8.

14. The method according to claim 13, wherein the useful crop is a transgenic crop or a crop treated by a genome editing technique.
